(19) United States
(12) Patent Application Publication
(10) Pub. No.: US 2010/0015220 A1
(43) Pub. Date: Jan. 21, 2010

(54) NIACIN AND NSAID COMBINATION THERAPY

(76) Inventors: John R. WETTERAU, Dexter, MI (US); Lingyu Zhu, Shanghai (CN); Robert A. Scott, Rouffiac-Tolosan (FR); Constance H. Keyserling, Toulouse (FR); Jean-Louis H. Dasseux, Toulouse (FR); Daniela Carmen Oniciu, Balma (FR); Pierre Autant, Malicome (FR); Roger Kravtzoff, Algans (FR); Catherine Castan, Orlienas (FR); Hervé Guillard, Villeurbanne (FR)

Correspondence Address: JONES DAY, 222 EAST 41ST ST, NEW YORK, NY, 10017, US

(21) Appl. No.: 12/469,653

(22) Filed: May 20, 2009

Related U.S. Application Data
(60) Provisional application No. 61/054,795, filed on May 20, 2008.
(60) Provisional application No. 61/102,335, filed on Oct. 2, 2008.

(51) Int. Cl. A61K 9/48; A61K 9/20; A61K 31/455; A61K 9/50; A61K 31/60; A61P 25/34

(52) U.S. Cl. 424/452; 514/161; 424/465; 514/356; 424/490; 514/165

(57) ABSTRACT

Provided are pharmaceutical compositions and methods for preventing or reducing niacin-induced flushing comprising an aspirin component and a niacin component having different release profiles. Also provided are methods and compositions for preventing or reducing niacin-induced flushing comprising niacin, aspirin and a lipid-lowering drug other than niacin.

This application claims the benefit of U.S. Provisional Application No. 61/054,795, filed May 20, 2008 and U.S. Provisional Application No. 61/102,335, filed Oct. 2, 2008, all of which are incorporated herein by reference in their entireties.

### FIELD

Provided are pharmaceutical compositions and formulations of one or more lipid-modulating drugs, particularly nicotinic acid with or without a second lipid-modulating agent, and NSAIDs. In one embodiment, the pharmaceutical compositions and formulations decrease adverse effects caused by these lipid-modulating drugs. In another embodiment, provided are specific dosing regimens of niacin and aspirin that decrease niacin-mediated flushing. In yet another embodiment, provided are specific dosing regimens of niacin, aspirin and lipid modulators that decrease niacin-mediated flushing. In yet another embodiment, methods for administration of combination formulations are provided.

### BACKGROUND

Niacin is one of the oldest drugs used to treat dyslipidemia and atherosclerosis and is the most versatile in that it favorably affects all lipid parameters (Altschul et al., Arch Biochem Biophys 54:558-559, 1955; Knopp, Am J Cardiol 82:24U-28U, 1998). Niacin is known to increase HDL-C levels (10% to 40%); it lowers the levels of triglycerides by 35% to 45% and reduces LDL-C levels by 20% to 30% (Knopp et al., Metabolism 34:642-650, 1985; Vega and Grundy, Arch Int Med 154:73-82, 1994; Martin-Jadraque, Arch Int Med 156:1081-1088, 1996). It also significantly reduces Lp(a) levels by about 40% (Carlson et al., Arch Int Med 226:271-276, 1989).

The mechanism of action of niacin is based on its multiple effects on lipoprotein metabolism. In adipose tissue, niacin inhibits the lipolysis of triglycerides by hormone-sensitive lipase, which reduces transport of free fatty acids to the liver and decreases hepatic triglyceride synthesis (Grundy et al., J Lipid Res 22:24-36, 1981). In the liver, niacin reduces triglyceride synthesis by inhibiting both the synthesis and etherification of fatty acids (Jin et al., Arterioscler Thromb Vasc Biol 19:1051-1059, 1999). Reduction of triglyceride synthesis reduces hepatic VLDL production, which accounts for the reduced LDL levels. Niacin also enhances LPL activity, which promotes the clearance of chylomicrons and VLDL triglycerides. Raising of HDL-C levels is caused by decreasing the fractional clearance of apoA-I in HDL (Blum et al., J Clin Invest 60:795-807, 1977). This effect is due to a reduction in the hepatic clearance of HDL-apoA-I, thereby increasing the apoA-I content of plasma and augmenting reverse cholesterol transport (Jin et al., Arterioscler Thromb Vasc Biol 17:2020-2028, 1997).

Based on its pharmacological profile, niacin is indicated for hypertriglyceridemia and/or elevated LDL-C levels. It is also useful for subjects affected by hypertriglyceridemia and/or low HDL-C levels.

There are two commonly available forms of niacin for the treatment of dyslipidemia and cardiovascular disease. Crystalline niacin (regular or immediate release) refers to niacin tablets that dissolve quickly after ingestion. Extended-release niacin refers to preparations that continuously release niacin for about 6 to 8 hours after ingestion.

One of the most widespread adverse effects of niacin that limits subject compliance is flushing. Studies have indicated that the flushing reaction is initiated by release of prostaglandin D (Stem et al., Clin Pharmacol Ther 50:66-70, 1991). It was shown that mice lacking prostaglandin D₂ and prostaglandin E₂ receptors had reduced flushing responses (Benyo et al., J Clin Invest 115(12):3634-3640, 2005). Cheng et al. reported that prostaglandin receptors are involved in nicotinic acid-induced flushing in humans (Cheng et al., PNAS USA 103(17):6682-6687, 2006.)

The use of immediate release niacin is associated with very high levels of flushing. This has been managed in the past by formulations, called sustained release or long acting niacin, which spread the release of niacin over more than 12 hours. Because of the reduction in plasma levels these formulations cause less flushing however these sustained release formulations were subsequently shown to cause unacceptable levels of liver toxicity. Subsequent niacin formulations, called extended release niacin, which spread the niacin release over up to 12 hours have been associated with less liver toxicity but still suffer from an unacceptable level of flushing. Another approach to reduce flushing is to dose the patient with aspirin.

Several non-steroidal anti-inflammatory drugs (NSAIDs) have been shown to inhibit the synthesis of prostaglandins by blocking the enzymes involved in prostaglandin synthesis. Among the NSAIDs, in clinical use are aspirin, ibuprofen, naproxen, phenylbutazone, indomethacin and flufenamic acid.

Aspirin has been shown to significantly reduce the cardiovascular risk. Aspirin is indicated to reduce the combined risk of death and nonfatal stroke in patients who have had ischemic stroke or transient ischemia of the brain due to fibrin platelet emboli; reduce the risk of vascular mortality in patients with a suspected acute MI; reduce the combined risk of death and nonfatal MI in patients with a previous MI or unstable angina pectoris; and reduce the combined risk of MI and sudden death in patients with chronic stable angina pectoris. Aspirin is indicated for patients who have undergone revascularization procedures (i.e., CABG, PTCA, or carotid endarterectomy) when there is a preexisting condition for which aspirin is already indicated. Also, aspirin is indicated for the relief of the signs and symptoms of rheumatoid arthritis, juvenile rheumatoid arthritis, osteoarthritis, spondyloarthropathies, and arthritis and pleurisy associated with SLE. In addition, aspirin has been proven to reduce the flushing induced by nicotinic acid in guinea pigs at doses of 50, 100 or 200 mg/kg (Anderson et al., Acta Pharmacol Toxicol 41:1-10, 1977) and in humans at a total dose of 975 mg administered in divided doses of 650 mg at 1 hr and 325 mg at 0.5 hr prior to a high dose of nicotinic acid (Wilken et al., Clin Pharmacol Ther 31:478-482, 1982).

U.S. Pat. No. 5,981,555 discloses pharmaceutical compositions for administration of niacin having reduced capacity to provoke a flushing reaction in a subject. The patent teaches niacin and aspirin dosing regimens that use extended release aspirin that provides the subject about 10 mg aspirin per hour. The individual is pretreated with aspirin 2-12 hours prior to niacin therapy. The preferred daily dose of aspirin is 40-80 mg. Extended release aspirin is given over a period of 8-10 hours.

The patent teaches that higher doses of aspirin are not necessarily more effective than lower doses. This may in part be due to the known ability of aspirin to interfere with niacin metabolism.

It is well known that the effect of a drug varies with the concentration of the drug at its site of action. Typically, as the concentration of a drug is increases, it will approach a concentration where it has maximum efficacy, and further increases in its concentration will not be any more effective. In some cases, the dose-response relationship can be more complex, for example, further increases beyond the concentration that produces the maximal effect may actually result in less effect. This latter example has a U shaped dose response relationship. In addition, when choosing an appropriate dose, one needs to account the tolerability and safety of a drug, which are expected to decrease as the dose is increased. In many cases, these factors will limit the dose. Thus, it is important to define the dose-response relationship of a drug to maximize the therapeutic benefit, while minimizing unwanted side effects.

### SUMMARY

In one aspect, provided are pharmaceutical compositions comprising time managed absorption/distribution of niacin and an NSAID that reduce niacin-induced flushing. NSAIDs suitable for pharmaceutical compositions provided herein include, but are not limited to, aspirin, ibuprofen, indomethacin, phenylbutazone and naproxen.

In one embodiment, provided are pharmaceutical composition of time managed administration/ distribution/absorption of aspirin (slow released aspirin) and niacin (delayed and extended released niacin) resulting in lower side effect.

In one embodiment, provided are pharmaceutical composition of time managed administration/ distribution/absorption of aspirin (slow released aspirin) and niacin (delayed and extended released niacin) resulting in reduced niacin-induced flushing.

In one embodiment, provided are pharmaceutical composition of time managed administration/distribution/absorption of aspirin (slow released aspirin) and niacin (delayed and extended released niacin) resulting in clinical benefits for treatment of atherosclerosis.

In one embodiment, provided are pharmaceutical composition of time managed administration/ distribution/absorption of aspirin (slow released aspirin) and niacin (delayed and extended released niacin) resulting in clinical benefits for treatment of dyslipidemia.

In one aspect, provided are pharmaceutical compositions comprising niacin and an NSAID or equivalent that reduce niacin-induced flushing. NSAIDs suitable for pharmaceutical compositions provided herein include, but are not limited to, aspirin, sodium salicylate, choline magnesium trisalicylate, salsalate, diflunisal, sulfasalazine, olsalazine, acetaminophen ibuprofen, naproxen, flurbiprofen, ketoprofen, fenoprofen, oxaprozin, indomethacin, sulindac, tolmetin, diclofenac, ketorolac, phenylbutazone, mefanamic acid, meclofenamic acid, piroxicam, meloxicam, nabumetone, rofecoxib, celecoxib, etodolac and nimesulide. Also prostaglandin receptor blockers, including but not limited to, laropiprant or compounds disclosed in the U.S. Patent Publication Nos. 2004/0229844 and 2005/0154044 can be employed. In one embodiment, prostaglandin D2 receptor blocker is MK-0524 (Merck & Co.)

In one embodiment, provided are pharmaceutical compositions comprising niacin and aspirin that reduce niacin-induced flushing.

In one embodiment, provided are pharmaceutical compositions comprising niacin and aspirin that reduce niacin-induced flushing comprising a total niacin daily dose of about 100 to about 3000 mg. In another embodiment, the total niacin daily dose of about 125 to about 2500 mg. In one embodiment, the niacin is in a extended and/or delayed release form.

In one embodiment, provided are pharmaceutical compositions comprising niacin and aspirin that reduce niacin-induced flushing, wherein the total aspirin daily dose is about 80 to about 2000 mg. In one embodiment, provided are pharmaceutical compositions comprising niacin and aspirin that reduce niacin-induced flushing, wherein the total aspirin daily dose is of about 80 to about 500 mg. In yet another embodiment, provided are pharmaceutical compositions comprising niacin and aspirin that reduce niacin-induced flushing, wherein the total aspirin daily dose is of about 80 to about 400 mg.

In one embodiment, provided is a formulation comprising niacin microparticles having a reduced capacity to provoke a flushing reaction in a subject, wherein the niacin microparticles have a specific niacin release profile, and aspirin microparticless having a specific aspirin release profile wherein aspirin is present in an amount effective to reduce a cutaneous flushing caused by the niacin, wherein this amount is about 80 to about 500 mg.

In another embodiment, provided are pharmaceutical compositions comprising niacin and aspirin that reduce niacin-induced flushing wherein the total aspirin daily dose is released at a rate of about 15-100 mg of aspirin/hour for a period of up to 16 hours. In another embodiment, provided are pharmaceutical compositions comprising niacin and aspirin that reduce niacin-induced flushing wherein the total aspirin daily dose is released at a rate of about 15-100 mg of aspirin/hour for a period of up to 24 hours.

In another embodiment, provided are pharmaceutical compositions comprising niacin and aspirin that reduce niacin-induced flushing wherein the total aspirin daily dose is released from the composition based on an aspirin release profile, wherein 80% of the aspirin dose is released over a period of time of about 2 to about 16 hours following administration of the composition. In another embodiment, the total daily dose of aspirin is released from the composition based on an aspirin release profile, wherein 90% of aspirin dose is released over a period of time of about 2 to about 16 hours following administration of the formulation. In yet another embodiment, the total daily dose of aspirin is released from the formulation based on an aspirin release profile, wherein aspirin concentration in plasma is greater than 5% of Cmax over a period of time of about 2 to about 16 hours following administration of the formulation. In still another embodiment, the total daily dose of aspirin is released from the composition based on an aspirin release profile, wherein aspirin concentration in plasma is greater than 10% of Cmax over a period of time of about 2 to about 16 hours following administration of the formulation.

In yet another embodiment, provided are pharmaceutical compositions comprising niacin and aspirin that reduce niacin-induced flushing wherein a subject is predosed with aspirin about 2 to about 16 hours before initiation of niacin therapy. In another embodiment, aspirin dosing includes 1-7 days of aspirin pretreatment. Such pretreatment may use an immediate or extended release aspirin formulation other than the formulation.

In yet another embodiment, provided are pharmaceutical compositions comprising niacin and aspirin that reduce niacin-induced flushing wherein a subject is predosed on the day of niacin therapy with an aspirin regimen, wherein about 90% of niacin AUC is not released until after a period of time of about 1 to about 12 hours of predosing with aspirin. In another embodiment, about 80% of niacin AUC is not released until after a period of time of about 1 to about 12 hours of predosing with aspirin. In another embodiment, the plasma concentration of niacin is less than 10% of Cmax until after a period of time of about 1 to about 12 hours of predosing with aspirin. In yet another embodiment, the plasma concentration of niacin is less than 20% of Cmax until after a period of time of about 1 to about 12 hours of predosing with aspirin. In another embodiment, aspirin predosing includes 1-4 days of aspirin pretreatment. Such pretreatment may use an immediate or extended release aspirin formulation.

In one embodiment, provided is a combination formulation that decreases prostaglandin D₂, prostaglandin D₂ metabolites and/or Prostaglandin E metabolites to a certain level.

In another embodiment, provided is a combination formulation that reduces flushing or flushing intensity to a certain percent. In another embodiment, provided is a combination formulation containing niacin and aspirin, that reduces flushing to a certain percent. In one embodiment, flushing or flushing intensity is reduced by about 10 to about 80%. In one embodiment, flushing is reduced by about 10 to about 20%. In one embodiment, flushing is reduced by about 20 to about 40%. In one embodiment, flushing is reduced by about 40 to about 60%. In one embodiment, flushing is reduced by about 60 to about 80%.

In yet another embodiment, provided is a combination formulation that reduces aspirin side effects. In yet another embodiment, provided is a combination formulation containing niacin and aspirin that reduces aspirin side effects. In one embodiment, aspirin side effects are reduced by about 5 to about 80%. In one embodiment, aspirin side effects are reduced by about 5 to about 20%.In one embodiment, aspirin side effects are reduced by about 20 to about 40%. In one embodiment, aspirin side effects are reduced by about 40 to about 60%. In one embodiment, aspirin side effects are reduced by about 60 to about 80%.

In another embodiment, provided is a combination formulation that decreases the niacin treatment drop-out rate. In another embodiment, provided is a combination formulation containing niacin and aspirin that decreases the niacin treatment drop-out rate.

In another embodiment, provided is a combination formulation that allows the niacin titration rate to increase. In another embodiment, provided is a combination formulation containing niacin and aspirin that allows the niacin titration rate to increase. In one embodiment, the niacin titration rate is increased by about 20 to about 80%. In one embodiment, the niacin titration rate is increased by about 20 to about 40%. In one embodiment, the niacin titration rate is increased by about 40 to about 60%. In one embodiment, the niacin titration rate is increased by about 60 to about 80%.

In another embodiment, provided is a combination formulation that allows a patient to tolerate a higher dose of aspirin. In another embodiment, provided is a combination formulation containing niacin and aspirin that allows a patient to tolerate a higher dose of aspirin.

In one embodiment, provided is a combination formulation that allows a patient to tolerate a higher dose of niacin. In another embodiment, provided is a combination formulation containing niacin and aspirin that allows a patient to tolerate a higher dose of niacin.

In another embodiment, provided are pharmaceutical compositions comprising niacin/aspirin dosing regimens that reduce niacin-induced flushing, further comprising a lipid-lowering drug other than niacin.

In one embodiment, aspirin microparticles are mixed with niacin microparticles to obtain a formulation comprising two types of microparticles with different release profiles. In another embodiment, provided is a formulation comprising a first population of microparticles and a second population of microparticles, wherein the first population of microparticles is an aspirin formulation having a first release profile, and wherein the second population of microparticles is a niacin formulation having a second release profile, wherein the first population of microparticles and the second population of microparticles are mixed.

In one embodiment, provided is a formulation wherein pH sensitive microparticles are used to control release of aspirin and niacin. In one embodiment, aspirin is released in a pH independent fashion. In another embodiment, niacin is released in a pH independent fashion. In another embodiment, niacin is released in a pH dependent fashion, wherein release is slow at a pH below 5.5 and release is faster at a pH above about 5.5. In another embodiment, niacin release is slow at pH below 5.5. In another embodiment, the pH for release of the niacin is about 5.5 to about 8.0. In one embodiment, release of the niacin is faster at a pH that is above about 5.5. In one embodiment, release of the niacin is faster at a pH that is above about 6.0. In one embodiment, release of the niacin is faster at a pH that is above about 6.5. In one embodiment, release of the niacin is faster at a pH that is above about 7.0. In one embodiment, release of the niacin is faster at a pH that is above about 7.5. In one embodiment, release of the niacin is faster at a pH that is above about 8.0.

In one embodiment, provided is a formulation comprising a mixture of aspirin microparticles and niacin microparticles so that aspirin and niacin are kept physically separated. In one embodiment, the aspirin microparticles and the niacin microparticles are administered at the same time as one formulation (combination formulation), having a lag time between release of the drugs. In one embodiment, the formulation is administered at bedtime or in the evening.

In one embodiment, provided is a pharmaceutical composition, wherein one capsule or tablet is used to orally pretreat a patient with aspirin and then provide a patient with a niacin dose. In one embodiment, one capsule or tablet comprises microparticles of aspirin and microparticles of niacin having different dissolution profiles. Due to the lag time in niacin release, aspirin is released earlier, then niacin is released later, for example, 2-16 hours after release of the aspirin.

In yet another embodiment, provided are methods for reducing niacin-induced flushing comprising administering to a subject a pharmaceutical composition comprising a niacin/aspirin dosing regimen comprising a total daily dose of aspirin of about 80 to about 2000 mg. In yet another embodiment, provided are methods for reducing niacin-induced flushing comprising administering to a subject a pharmaceutical composition comprising a niacin/aspirin dosing regimen comprising a total daily dose of aspirin of about 80 to about 500 mg. In yet another embodiment, provided are methods for reducing niacin-induced flushing comprising administering to a subject a pharmaceutical composition comprising a niacin/aspirin dosing regimen comprising a total daily dose of aspirin of about 80 to about 400 mg.

In one embodiment, the methods provided herein comprise combination formulation, wherein the total aspirin daily dose is released at a rate of about 15-100 mg of aspirin/hour for a period of up to 16 hours. In one embodiment, the methods provided herein comprise a combination formulation containing niacin and aspirin, wherein the total aspirin daily dose is released at a rate of about 15-100 mg of aspirin/hour for a period of up to 24 hours.

In one embodiment, the methods provided herein comprise a niacin/aspirin pharmaceutical composition wherein the total aspirin daily dose is released from the composition based on an aspirin release profile, wherein 80% of aspirin dose is released over a period of time of about 2 to about 16 hours following administration of the composition. In another embodiment, the total daily dose of aspirin dose is released from the composition based on aspirin release profile, wherein 90% of aspirin dose is released over a period of time of about 2 to about 16 hours following administration of the composition. In yet another embodiment, the total daily dose of aspirin is released from the composition based on aspirin release profile, wherein aspirin concentration in plasma is greater than 5% of Cmax over a period of time of about 2 to about 16 hours following administration of the composition. In still another embodiment, the total daily dose of aspirin is released from the composition based on aspirin release profile, wherein aspirin concentration in plasma is greater than 10% of Cmax over a period of time of about 2 to about 16 hours following administration of the composition. In one embodiment, a period of time is up to 24 hours.

In another embodiment, the methods provided herein comprise a step of predosing a subject with an aspirin regimen on the day of niacin therapy, wherein about 80% of niacin AUC is not released until after a period of time of about 2 to about 16 hours of predosing with aspirin. In another embodiment, about 90% of niacin AUC is not released until after a period of time of about 2 to about 16 hours of predosing with aspirin. In another embodiment, the plasma concentration of niacin is less than 10% of Cmax until after a period of time of about 2 to about 16 hours of predosing with aspirin. In yet another embodiment, the plasma concentration of niacin is less than 20% of Cmax until after a period of time of about 2 to about 16 hours of predosing with aspirin.

In another embodiment, the methods provided herein comprise a step of predosing a subject with aspirin for 1-7 days before initiation of niacin therapy. Such pretreatment may use an immediate or extended release aspirin formulation.

In another embodiment, the methods provided herein comprise niacin/aspirin dosing regimens that reduce niacin-induced flushing, further comprising a lipid-lowering drug other than niacin.

In another embodiment, provided are methods for reducing at least one component of the flushing symptoms. In another embodiment, provided are methods for reducing at least one component of the flushing symptoms comprising administering to a patient a combination formulation containing niacin and aspirin. These symptoms include, but are not limited to, redness, warmth, tingling, itching, burning, fever-like sensation and crawling sensation of the skin.

In another embodiment, provided are methods for decreasing prostaglandin-related side effects. In another embodiment, provided are methods for decreasing prostaglandin-related side effects comprising administering to a patient a combination formulation containing niacin and aspirin.

In another embodiment, provided are methods for decreased discontinuation of niacin treatment. In another embodiment, provided are methods for decreased discontinuation of niacin treatment comprising administering to a patient a combination formulation containing niacin and aspirin. In yet another embodiment, provided are methods for increased patient compliance with niacin treatment. In another embodiment, provided are methods for increased patient compliance with niacin treatment comprising administering to a patient a combination formulation containing niacin and aspirin.

In another embodiment, provided are methods for prevention of aspirin hydrolysis prior to its release from an oral dosage form. In another embodiment, provided are methods for prevention of aspirin hydrolysis after ingestion but prior to its release from an oral dosage form. In another embodiment, provided are methods for prevention of aspirin hydrolysis prior to its release from an oral dosage form comprising administering to a patient an aspirin/niacin formulation provided herein, wherein aspirin microparticles and niacin microparticles have different release profiles. In yet another embodiment, provided are formulations in which aspirin is treated with excipients that produce microparticles with increased stability that delay, minimize or avoid hydrolysis until certain in vivo conditions apply.

In yet another embodiment, provided are oral dosage forms of aspirin and niacin, or pharmaceutical compositions containing aspirin and niacin, which protect against gastro-intestinal (GI) irritation and GI tract side-effects including, but not limited to, bleeding.

In yet another embodiment, provided are oral dosage forms of aspirin, or pharmaceutical compositions containing aspirin, which protect against gastro-intestinal (GI) irritation and GI tract side-effects including, but not limited to, dyspepsia, stomach pain, gastric or peptic erosion, ulceration or perforation, gastrointestinal bleeding nausea, vomiting, transient elevations of hepatic enzymes, hepatitis, Reye's Syndrome, pancreatitis.

In yet another embodiment, provided are oral dosage forms of aspirin and niacin, or pharmaceutical compositions containing aspirin and niacin, which protect against drug interactions due to aspirin. These oral dosage forms include, but not limited to, angiotensin converting enzyme (ACE) inhibitors, acetazolamide, anticoagulants, anticonvulsants, beta blockers, diuretics, hypoglycemics and uricosuric agents.

The pharmaceutical compositions and methods provided herein will be described in details below. All oral dosage forms are in the scope of the disclosure.

### DETAILED DESCRIPTION

Definitions

As used herein, the following terms shall have the following meaning:

The term “aspirin” refers to acetyl salicylic acid. The term “aspirin metabolites” includes, but is not limited to, salicylic acid, salicyluric acid, phenolic acid, gentisic acid, 2,3-dihydroxybenzoic acid, and 2,3,5-trihydroxybenzoic acid, acetylsalicylsalicilic acid and salicylsalicylic acid.

The term “niacin” refers to nicotinic acid. The term “niacin metabolites” includes, but is not limited to, nicotinuric acid, nicotinamide, 6-hydroxy-nicotinamide, nicotinamide-N-oxide, nicotinic acid mononucleotide, nicotinic acid adenine dinucleotide, N′-methylnicotinamide, N′-methyl-2-pyridone-5-carboxamide (2-py), N′-methyl-4-pyridone-3-carboxamide (4-py), and nicotinamide mononucleotide.

The terms “treat”, “treating” or “treatment” refer to alleviating, reducing, abrogating, or otherwise modulating a disease, disorder, risk factor for a disease and/or symptoms thereof, that is a therapeutic effect on an existing condition.

The term “therapeutically effective amount” refers to that amount of an active ingredient sufficient to improve one or more of the symptoms of the condition or disorder being treated as compared to those symptoms that occur without treatment.

The term “cardiovascular diseases” refers to heart, blood vessel, and blood circulation diseases, such as myocardial infarction, acute coronary syndrome, atherosclerosis, angina, ischemic reperfusion injury and other related disorders described herein and known to those in the art.

The term “dyslipidemia” refers to a disorder of lipoprotein metabolism, including lipoprotein overproduction or deficiency. Dyslipidemias may be manifested by elevation of the total cholesterol, the “bad” low-density lipoprotein (LDL) cholesterol, apoB containing lipoproteins, Lp(a) and/or the triglyceride concentrations, and/or a decrease in the “good” high-density lipoprotein (HDL) cholesterol concentration and/or apoAI containing lipoproteins in the blood.

The term “atherosclerosis” refers to a form of arteriosclerosis characterized by the deposition of atheromatous plaques containing cholesterol and lipids on the innermost layer of the walls of large and medium-sized arteries.

The term “combination therapy” refers to the use of more than one drug to treat or prevent a condition or conditions in a subject. Each component of the combination therapy may or may not be in the form of a pharmaceutical composition. The drugs may be used simultaneously or sequentially. In one embodiment, one or more of the oral dosage form agents are oral. In another embodiment, two or three of the components of the oral dosage forms are used simultaneously.

As used herein and unless indicated otherwise, “initiation of niacin therapy” means the dosing time point or the presence of administered niacin in blood.

As used herein and unless otherwise indicated, the term “microparticle” means one of the following: i) granules comprised of a chemically neutral core on which the active principle (amorph materials, microcrystals or micronized product) is deposited by various technologies such as, but not limited to, spray drying, extrusion-spheronization, all being surrounded by a coating comprised of a mixture of pharmaceutically acceptable excipients (mixtures of hydrophobic or hydrophilic polymers and waxes), as listed and whose characteristics are described by pharmacopeias, or ii) granules of active principle created by extrusion-speronization and mixed with extrusion excipients coated with a mixture of pharmaceutically acceptable excipients (mixtures of hydrophobic or hydrophilic polymers and waxes), as listed and whose characteristics are described by pharmacopeias, or iii) macrocrystals either per se or mixed/coated with extrusion or other excipients (mixtures of hydrophobic or hydrophilic polymers and waxes), as listed and whose characteristics are described by pharmacopeias. Formulations of this type are small particles of drug created by those technologies in the presence of coating elements. Examples are presented further herein, as well as excipients used in such preparations.

As used herein and unless otherwise indicated, the term “pharmaceutical composition” or “combination formulation” means physical mixtures having as active principles aspirin, niacin, NSAID compounds, statins, fibrates or cholesterol absorption inhibitors, bile acid sequestrants as well as their prodrugs. Preferably the pharmaceutical formulation or composition will comprise niacin and aspirin and optionally a lipid-lowering drug, or another active principle such as COX inhibitors, arachidonic acid pathway inhibitors, PGD2 receptor inhibitors, phospholypase-A2 inhibitors, PPAR activators, P2Y12 and P2Y13 ligands, and PCSK-9 inhibitors, selective PPAR alpha activators/agonists, dual PPAR alpha, gamma activator/agonists, dual PPAR alpha, delat activator/agonists or pan PPAR alpha, gamma, delta/beta agonists, and anti-diabetic, anti-obesity and anti-hypertensive agents.

Pharmaceutical compositions can also be comprised of mixtures of aspirin and/or niacin with microparticles having active principles, including but not limited to, COX inhibitors, arachidonic acid pathway inhibitors, PGD2 receptor inhibitors, phospholipase-A2 inhibitors, PPAR activators, P2Y12 and P2Y13 ligands, and PCSK-9 inhibitors. Pharmaceutical compositions can also contain as components beta-blockers, diuretics, ACE inhibitors, angiotensin-receptor blockers, calcium channel blockers, renin inhibitors and other cardiovascular drugs.

The term “pharmaceutical formulation” refers to a composition comprising an active ingredient and a suitable diluent, carrier, vehicle, or excipients suitable for administration to a subject. As used herein and unless otherwise indicated, a formulation is in the form of but not limited to, a capsule, a tablet, an effervescent tablet, a sachet, a syrup, all containing physically independent mixtures of particles, microparticles, and active ingredients. The term is also meant to encompass situations wherein the components of the combination therapy are in separate formulations. This term includes, but is not limited to oral, parenteral, mucosal and topical compositions as described below. The term is also meant to include formulations where a slow-or extended-release product is administered subsequently to an immediate-release product or conversely.

As used herein unless otherwise indicated, an “NSAID compound and an equivalent” means, but is not limited to, aspirin, sodium salicylate, choline magnesium trisalicylate, salsalate, diflunisal, sulfasalazine, olsalazine, acetaminophen ibuprofen, naproxen, flurbiprofen, ketoprofen, fenoprofen, oxaprozin, indomethacin, sulindac, tolmetin, diclofenac, ketorolac, phenylbutazone, mefanamic acid, meclofenamic acid, piroxicam, meloxicam, naburnetone, rofecoxib, celecoxib, etodolac and nimesulide, and their metabolites.

As used herein unless otherwise indicated, the term “modified release formulation” means a formulation conceived to act in two phases: the first one allows for the release of the active principle to be delayed for a predetermined time from dosing (called lag time), and the second is an extended release of the active principle. Both phases are strongly dependent on the nature and ratio of the excipients, as described in Example 3 herein.

As used herein unless otherwise indicated, an “extended release formulation” is defined as one in which the release of drug from the dosage form is retarded such that the plasma levels are sustained for a longer period of time. Many terms are used to describe extended-release products including modified-release, prolonged-release, controlled-release, controlled-delivery, slow-release, intermediate release, and sustained-release. These preparations, by definition, have a reduced rate of release of active substance. In general, these terms are interchangeable.

The term “over a period of time of about X hours to about Y hours” is meant to be over any period of time that is greater than about X hours and less than about Y hours. Similarly, “over a period of about X to about Y hours” is meant to be over any period of time that is greater than about X hours and less than about Y hours.

The term “statins” refers to a group of compounds, which inhibit cholesterol synthesis. In one embodiment, statins are HMG-CoA reductase inhibitors. Examples of statins include lovastatin, simvastatin, pravastatin, fluvastatin, cerivastatin, ezetimibe/simvastatin, pitavastatin, rosuvastatin, atorvastatin or combinations thereof (Goodman & Gilman's The Pharmacological Basis of Therapeutics, Ed. J. Hardman, L. Limbird and A. Goodman Gilman, McGraw-Hill Medical Publishing Division, 10^{th} Edition, 2001, pp. 982-987).

As used herein and unless otherwise indicated, the term “fibrate” means an amphipatic carboxylic acid that is used for the treatment of metabolic disorders, mainly hypercholesterolemia, such as but not limited to benzafibrate, fenofibrate, clofibrate, gemfibrozil, cipofibrate, selective PPAR alpha activators/agonists, dual PPAR alpha, gamma activator/agonists, dual PPAR alpha, delat activator/agonists or pan PPAR alpha, gamma, delta/beta agonists.

As used herein and unless otherwise stated, the term “cholesterol absorption inhibitors” refers to compounds that prevent the uptake of cholesterol from the small intestine into the circulatory system, such as but not limited to ezetimibe.

As used herein and unless otherwise stated, the term “bile acid sequestrants” refers to compounds, particularly resins that bind some bile components and cholesterol in the gastro-intestinal tract by disrupting their entherohepatic circulation and sequestering them and preventing them from reabsorption in the gut. Examples of bile acid sequestrants are cholestyramine, colestipol, colestilan, etc.

As used herein and unless otherwise indicated, a pharmaceutical composition or formulation that “substantially” comprises a compound means that the composition contains more than about 50% by weight, preferably more than about 70% by weight, more preferably more than about 80% by weight of the compound or its acceptable prodrug or its pharmaceutically acceptable salts.

As used herein and unless otherwise indicated, the term “acceptable prodrug” means a derivative of a compound that can hydrolyze, oxidize, or otherwise react under biological conditions (in vitro or in vivo) to provide the compound. Examples of prodrugs include, but are not limited to, compounds that comprise biohydrolyzable moieties such as biohydrolyzable amides, biohydrolyzable esters, biohydrolyzable carbamates, biohydrolyzable carbonates, biohydrolyzable ureides, and biohydrolyzable phosphate analogues. Other examples of prodrugs include compounds that comprise oligonucleotides, peptides, lipids, aliphatic and aromatic groups, or NO, NO₂, ONO, and ONO₂ moieties. Prodrugs can typically be prepared using well known methods, such as those described in Burger's Medicinal Chemistry and Drug Discovery, pp. 172, 178, 949, 982 (Manfred E. Wolff ed., 5th ed. 1995), and Design of Prodrugs (H. Bundgaard ed., Elselvier, N.Y. 1985). As used herein and unless otherwise indicated, the terms “biohydrolyzable amide,” “biohydrolyzable ester,” “biohydrolyzable carbamate,” “biohydrolyzable carbonate,” “biohydrolyzable ureide,” “biohydrolyzable phosphate” mean an amide, ester, carbamate, carbonate, ureide, or phosphate, respectively, of a compound that either: 1) does not interfere with the biological activity of the compound but can confer upon that compound advantageous properties in vivo, such as uptake, duration of action, or onset of action; or 2) is biologically inactive but is converted in vivo to the biologically active compound. Examples of biohydrolyzable esters include, but are not limited to, lower alkyl esters, lower acyloxyalkyl esters (such as acetoxylmethyl, acetoxyethyl, aminocarbonyloxy-methyl, pivaloyloxymethyl, and pivaloyloxyethyl esters), lactonyl esters (such as phthalidyl and thiophthalidyl esters), lower alkoxyacyloxyalkyl esters (such as methoxycarbonyloxy-methyl, ethoxycarbonyloxy-ethyl and isopropoxycarbonyloxyethyl esters), alkoxyalkyl esters, choline esters, and acylamino alkyl esters (such as acetamidomethyl esters). Examples of biohydrolyzable amides include, but are not limited to, lower alkyl amides, a amino acid amides, alkoxyacyl amides, and alkylaminoalkyl-carbonyl amides. Examples of biohydrolyzable carbamates include, but are not limited to, lower alkylamines, substituted ethylenediamines, aminoacids, hydroxyalkylamines, heterocyclic and heteroaromatic amines, and polyether amines.

As used herein and unless otherwise indicated, the phrase “pharmaceutically acceptable salt(s),” as used herein includes, but is not limited to, salts of acidic or basic groups that may be present in the compounds of the invention. Compounds that are basic in nature are capable of forming a wide variety of salts with various inorganic and organic acids. The acids that may be used to prepare pharmaceutically acceptable acid addition salts of such basic compounds are those that form non-toxic acid addition salts, i.e., salts containing pharmacologically acceptable anions, including but not limited to sulfuric, citric, maleic, acetic, oxalic, hydrochloride, hydrobromide, hydroiodide, nitrate, sulfate, bisulfate, phosphate, acid phosphate, isonicotinate, acetate, lactate, salicylate, citrate, acid citrate, tartrate, oleate, tannate, pantothenate, bis-tartrate, ascorbate, succinate, maleate, gentisinate, fumarate, gluconate, glucaronate, saccharate, formate, benzoate, glutamate, methanesulfonate, ethanesulfonate, benzenesulfonate, p-toluenesulfonate and palmoate (i.e., 1,1′-methylene-bis-(2-hydroxy-3-naphthoate)) salts. Compounds of the invention that include an amino moiety also can form pharmaceutically acceptable salts with various amino acids, in addition to the acids mentioned above. Compounds of the invention that are acidic in nature are capable of forming base salts with various pharmacologically acceptable cations. Examples of such salts include alkali metal or alkaline earth metal salts and, particularly, calcium, magnesium, sodium lithium, zinc, potassium, and iron salts.

The term “anti-inflammatory drugs” refers to a group of compounds that counteract inflammation. As known by those in the art, inflammation can include, but is not limited to, a local response to cellular injury that is marked by capillary dilation, leukocyte infiltration, redness, heat, and/or pain. The term “an anti-inflammatory drug” encompasses anti-inflammatory steroids and non-steroidal anti-inflammatory agents (NSAIDS). (Goodman & Gilman's The Pharmacological Basis of Therapeutics, Ed. J. Hardman, L. Limbird and A. Goodman Gilman, McGraw-Hill Medical Publishing Division, 10^{th} Edition, 2001, pp. 687-715). In the large sense, other compounds can be included as anti-inflammatory agents, for example COX inhibitors, arachidonic acid pathway inhibitors, PGD2 receptor inhibitors, phospholyiase-A2 inhibitors, antioxidant drugs or drugs which inhibit the production of reactive oxygen species, PPAR activators, P2Y12 and P2Y13 ligands, and PCSK-9 inhibitors, beta-blockers, diuretics, ACE inhibitors, angiotensin-rhenin blockers and other cardiovascular drugs and therefore those compounds are further included in a larger definition of NSAIDS.

The term “hypolipemic amount” of niacin includes an amount which initially may be less that the amount which produces clinically significant reduction in plasma lipid or lipoprotein levels.

The term “flush-reducing regimen of aspirin” refers to a regimen of aspirin doses in any dosage form or composition which is effective to reduce niacin-induced flushing (including burning, itching, crawling sensation, pain, reddening of the skin, and/or fever like sensation) prior to, during or after niacin administration. The term “flush-reducing amount and dosage form of aspirin” refers to an amount which, in its dosage form, reduces niacin-induced flushing (including burning, itching, crawling sensation, pain, reddening of the skin, and/or fever like sensation) when administered prior to, concurrently, and/or subsequent to niacin, the amount being substantially below that used to treat systemic symptoms of inflammation such as pain and fever in that dosage form. It will be apparent that the total amount of aspirin in a slow or extended release formulation which satisfies the definition above, if formulated in an immediate release formulation, may be sufficient to produce an anti-inflammatory response.

The term “aspirin dose” refers to a dose with an acceptable balance of efficacy and side effects.

The term “evening administration” refers to administration between 4:30 pm and 2:00 am.

The term “lag time” refers to the time between dosing and initiation of the niacin therapy or the time between dosing and the appearance of a certain percentage of administered niacin in blood.

The term “predosing” refers to dosing a patient with two separate dosage forms (any type of formulation including, but not limited to, tablets, capsules, sachets) or a subject takes one dosage form (as a pharmaceutical combination in the form of a formulation including, but not limited to, tablets, capsules, sachets wherein the release of one drug component is delayed by a lag time, which varies. In one embodiment, the time varies from 2 to 16 hours.

The term “plasma level” means the concentration of a drug in the blood at any particular time, regardless of the therapeutic response. This value is essentially the same as the serum level or blood level of the drug and the concept can be used interchangeably.

### DETAILED DESCRIPTION OF THE FIGURES

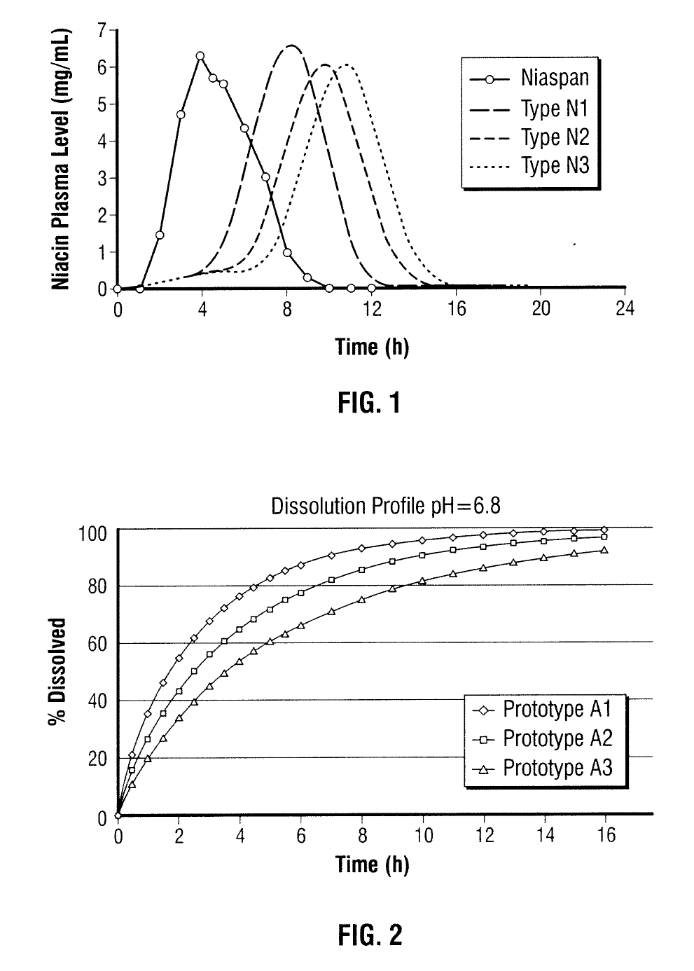

FIG. 1 shows simulated in vivo absorption of niacin formulations with increasing Tmax values.

FIG. 2 shows target dissolution profiles at pH 6.8 for each type of Aspirin SR 81 mg capsule.

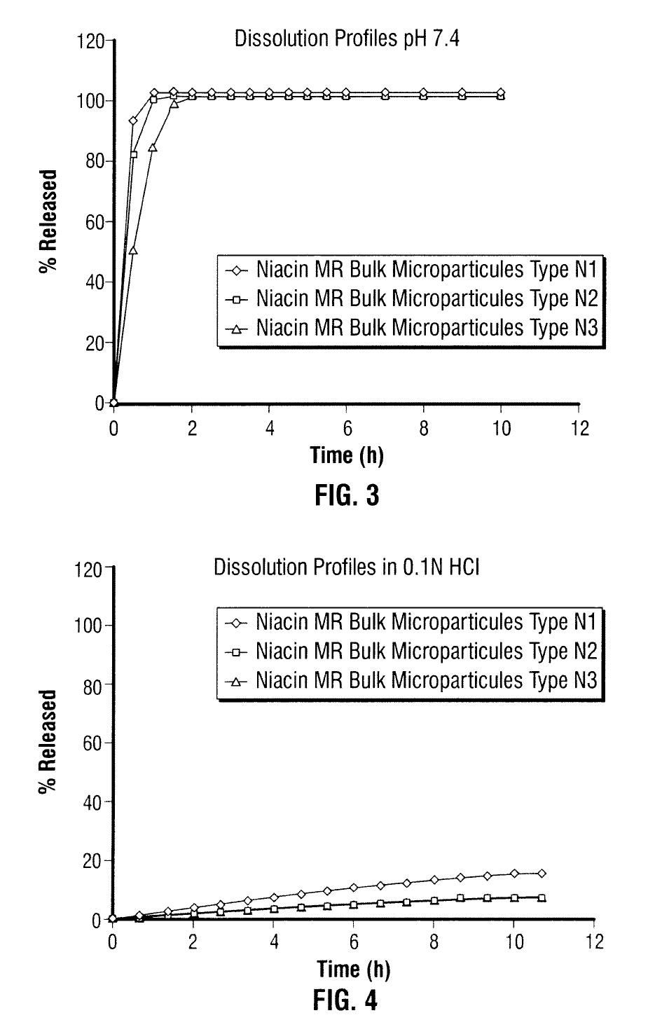

FIG. 3 shows niacin prototypes dissolution profiles at pH 7.4.

FIG. 4 shows niacin prototypes dissolution profiles in 0.1 N HCL.

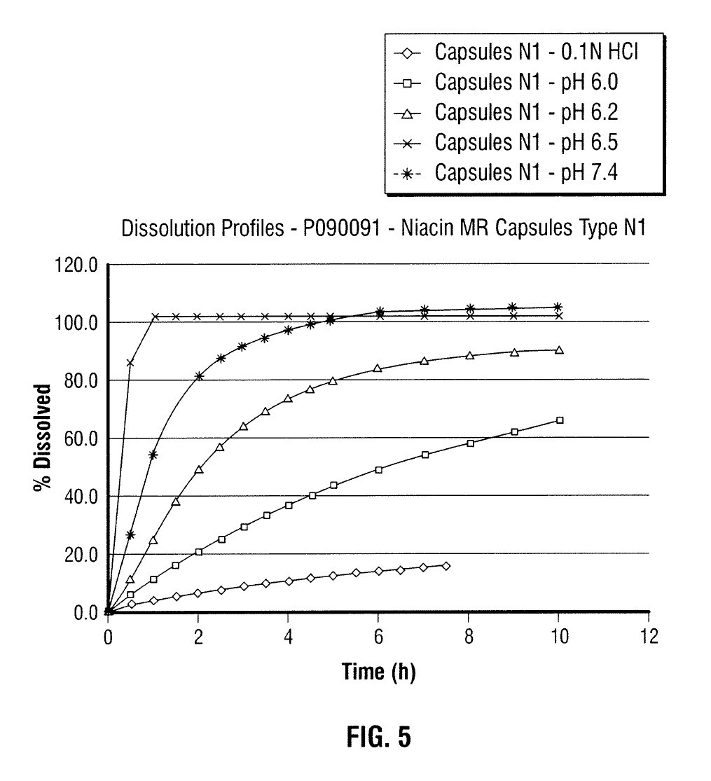

FIG. 5 shows niacin type N1 dissolution profile.

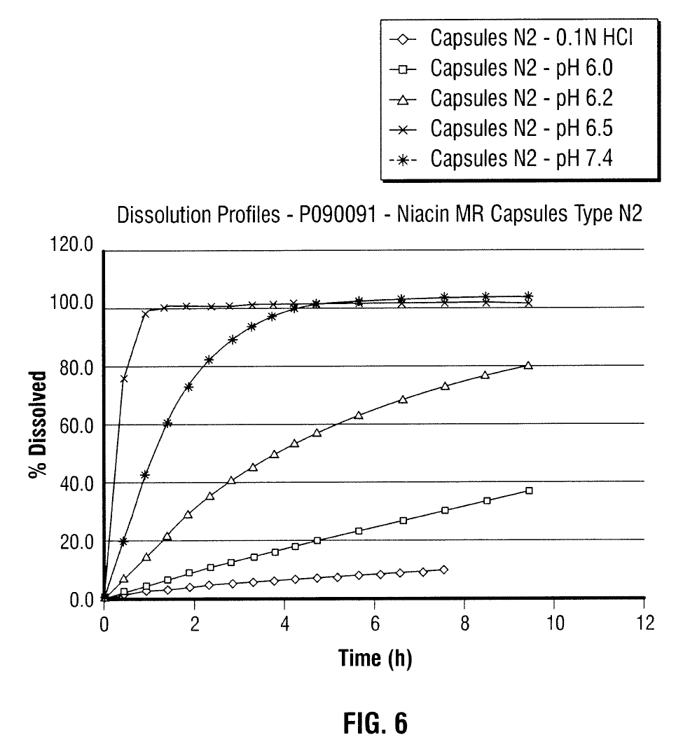

FIG. 6 shows niacin type N2 dissolution profile.

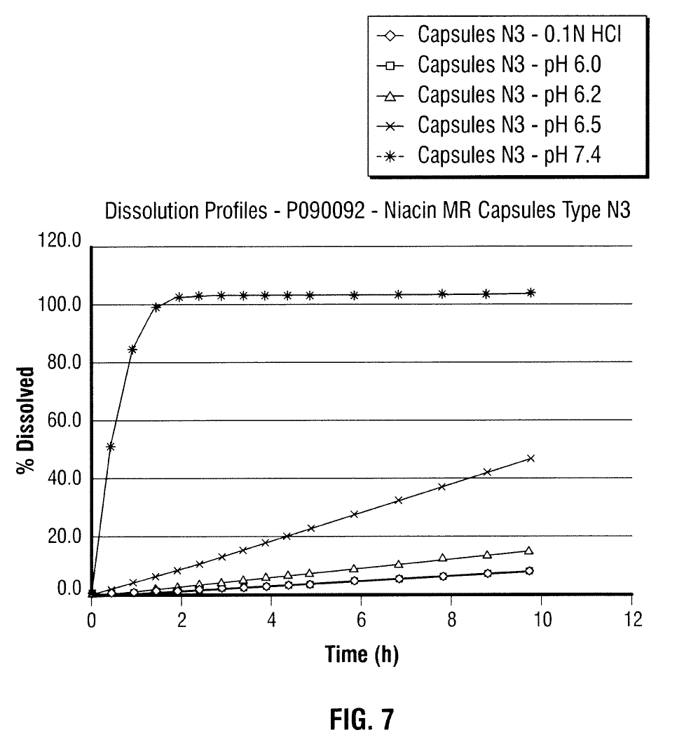

FIG. 7 shows niacin type N3 dissolution profile.

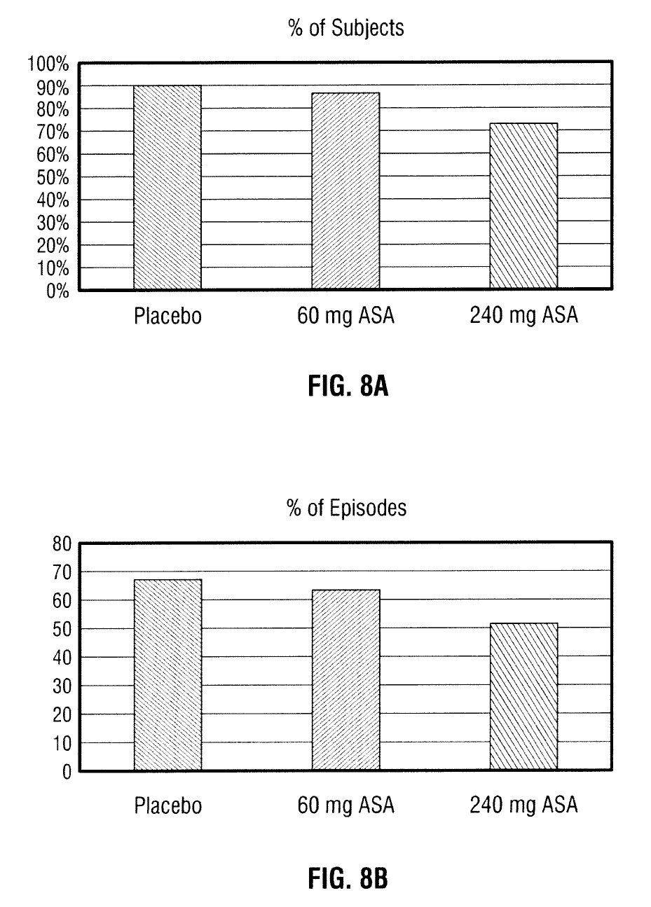

FIG. 8A shows incidence of flushing according to percent of subjects who received placebo, 60 mg of ASA and 240 mg of ASA. FIG. 8B shows incidence of flushing according to number of episodes when subjects received placebo, 60 mg of ASA and 240 mg of ASA.

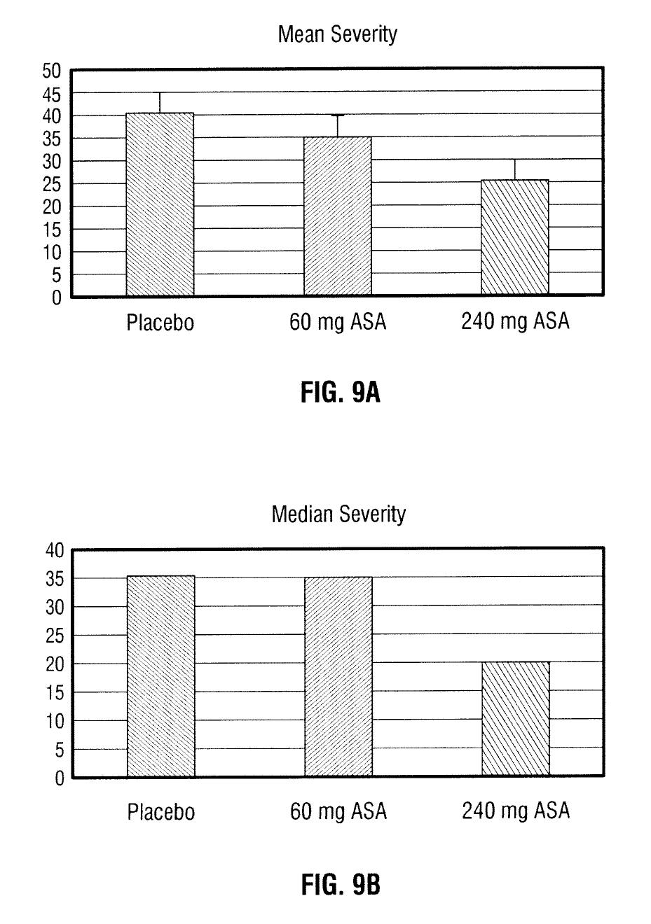

FIGS. 9A and 9B show mean severity (9A) and median severity (9B) of flushing when subjects received placebo, 60 mg of ASA and 240 mg of ASA.

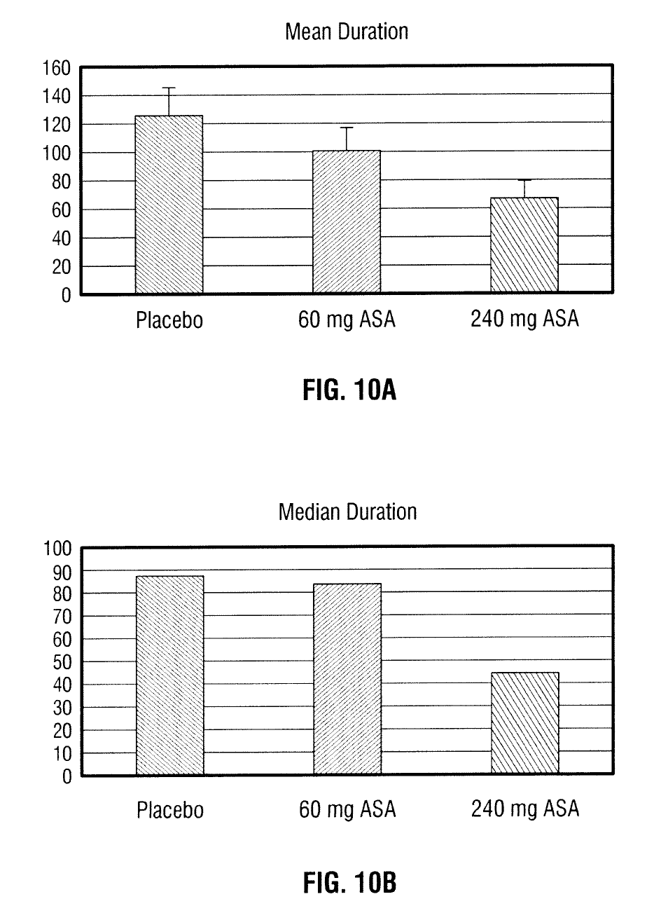

FIGS. 10A and 10B show mean duration (10A) and median duration (10B) of flushing when subjects received placebo, 60 mg of ASA and 240 mg of ASA.

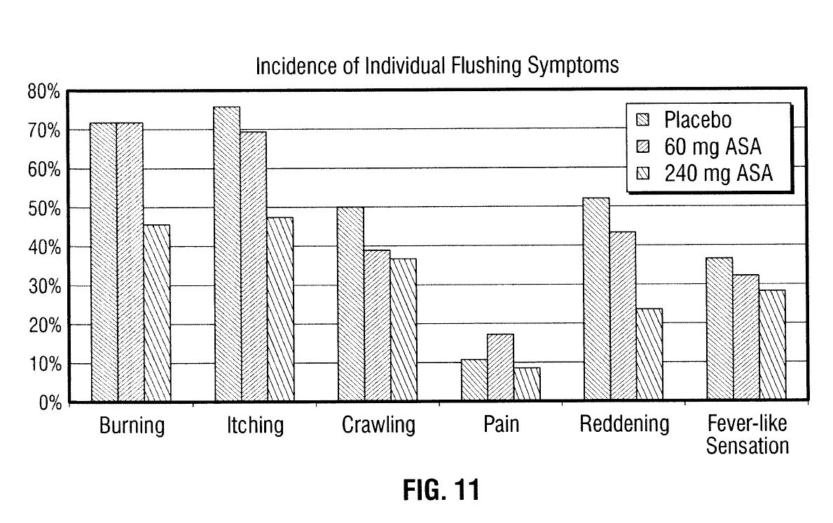

FIG. 11 shows an influence of placebo, 60 mg of ASA and 240 mg of ASA on incidence of individual flushing symptoms.

### NIACIN DOSING

In one aspect, provided are pharmaceutical compositions comprising niacin and an NSAID that reduce niacin-induced flushing.

In one embodiment, the total daily dose of niacin is about 100 mg to about 3000 mg. In another embodiment, the total daily dose of niacin is about 125 mg to about 2500 mg. In another embodiment, the total daily dose of niacin is about 250 mg to about 2500 mg. In another embodiment, the total daily dose of niacin is about 500 mg to about 2500 mg. In another embodiment, the total daily dose of niacin is about 200 mg to about 2000 mg. In another embodiment, the total daily dose of niacin is about 500 mg to about 2000 mg. In another embodiment, the total daily dose of niacin is about 1000 mg to about 2000 mg. In yet another embodiment, the total daily dose of niacin is about 250 mg to about 750 mg. In another embodiment, the total daily dose of niacin is about 250 mg to about 500 mg. In another embodiment, the total daily dose of niacin is about 400 mg to about 500 mg.

In certain embodiments, the total daily dose of niacin is about 100 mg. In certain embodiments, the total daily dose of niacin is about 125 mg. In certain embodiments, the total daily dose of niacin is about 250 mg. In certain embodiments, the total daily dose of niacin is about 333 mg. In certain embodiments, the total daily dose of niacin is about 375 mg. In certain embodiments, the total daily dose of niacin is about 500 mg. In certain embodiments, the total daily dose of niacin is about 750 mg. In certain embodiments, the total daily dose of niacin is about 1000 mg. In certain embodiments, the total daily dose of niacin is about 1250 mg. In certain embodiments, the total daily dose of niacin is about 1500 mg. In certain embodiments, the total daily dose of niacin is about 2000 mg. In certain embodiments, the total daily dose of niacin is about 2250 mg. In certain embodiments, the total daily dose of niacin is about 2500 mg. In certain embodiments, the total daily dose of niacin is about 3000 mg.

In one embodiment, pharmaceutical compositions provided herein comprise niacin that is in an extended release form. In another embodiment, pharmaceutical compositions provided herein comprise niacin that is in an immediate release form.

In another embodiment, in the embodiments described therein, less than about 78% of niacin AUC is released from the composition between about 0 hours and about 8 hours following ingestion.

Aspirin Dosing

In one aspect, provided are pharmaceutical compositions comprising niacin and an NSAID that reduce niacin-induced flushing. NSAIDS suitable for pharmaceutical compositions provided herein include, but not limited to, aspirin, ibuprofen, indomethacin, phenylbutazone and naproxen. In one embodiment, provided are pharmaceutical compositions comprising niacin and aspirin.

In one embodiment, the total daily dose of aspirin is about 80 mg to about 2000 mg. In another embodiment, the total daily dose of aspirin is about 80 mg to about 500 mg. In another embodiment, the total daily dose of aspirin is about 80 mg to about 400 mg. In another embodiment, the total daily dose of aspirin is about 80 mg to about 320 mg. In another embodiment, the total daily dose of aspirin is about 80 mg to about 240 mg. In another embodiment, the total daily dose of aspirin is about 80 mg to about 200 mg. In another embodiment, the total daily dose of aspirin is about 80 mg to about 160 mg. In another embodiment, the total daily dose of aspirin is about 80 mg to about 140 mg. In another embodiment, the total daily dose of aspirin is about 80 mg to about 130 mg. In another embodiment, the total daily dose of aspirin is about 80 mg to about 100 mg. In another embodiment, the total daily dose of aspirin is about 80 mg to about 81 mg. In certain embodiments, the total daily dose of aspirin is about 80 mg. In certain embodiments, the total daily dose of aspirin is about 81 mg.

In one embodiment, the total daily dose of aspirin is about 80 mg to about 2000 mg. In one embodiment, the total daily dose of aspirin is about 80 mg to about 500 mg. In another embodiment, the total daily dose of aspirin is about 80 mg to about 400 mg. In another embodiment, the total daily dose of aspirin is about 80 mg to about 320 mg. In another embodiment, the total daily dose of aspirin is about 80 mg to about 240 mg. In another embodiment, the total daily dose of aspirin is about 80 mg to about 200 mg. In another embodiment, the total daily dose of aspirin is about 100 mg to about 160 mg. In another embodiment, the total daily dose of aspirin is about 100 mg to about 140 mg. In another embodiment, the total daily dose of aspirin is about 110 mg to about 130 mg. In certain embodiments, the total daily dose of aspirin is about 120 mg.

In one embodiment, the total daily dose of aspirin is about 120 mg to about 2000 mg. In another embodiment, the total daily dose of aspirin is about 120 mg to about 500 mg. In another embodiment, the total daily dose of aspirin is about 120 mg to about 400 mg. In another embodiment, the total daily dose of aspirin is about 120 mg to about 325 mg. In another embodiment, the total daily dose of aspirin is about 120 mg to about 240 mg. In another embodiment, the total daily dose of aspirin is about 140 mg to about 200 mg. In another embodiment, the total daily dose of aspirin is about 150 mg to about 170 mg. In another embodiment, the total daily dose of aspirin is about 160 mg to about 162 mg. In certain embodiments, the total daily dose of aspirin is about 160 mg. In certain embodiments, the total daily dose of aspirin is about 162 mg.

In one embodiment, the total daily dose of aspirin is about 160 mg to about 2000 mg. In another embodiment, the total daily dose of aspirin is about 160 mg to about 500 mg. In another embodiment, the total daily dose of aspirin is about 160 mg to about 325 mg. In another embodiment, the total daily dose of aspirin is about 180 mg to about 300 mg. In another embodiment, the total daily dose of aspirin is about 200 mg to about 260 mg. In another embodiment, the total daily dose of aspirin is about 220 mg to about 240 mg. In another embodiment, the total daily dose of aspirin is about 240 mg to about 243 mg. In certain embodiments, the total daily dose of aspirin is about 240 mg. In certain embodiments, the total daily dose of aspirin is about 243 mg.

In one embodiment, the total daily dose of aspirin is about 160 mg to about 2000 mg. In another embodiment, the total daily dose of aspirin is about 160 mg to about 500 mg. In another embodiment, the total daily dose of aspirin is about 160 mg to about 380 mg. In another embodiment, the total daily dose of aspirin is about 200 mg to about 360 mg. In another embodiment, the total daily dose of aspirin is about 260 mg to about 360 mg. In another embodiment, the total daily dose of aspirin is about 300 mg to about 340 mg. In another embodiment, the total daily dose of aspirin is about 320 mg to about 324 mg. In certain embodiments, the total daily dose of aspirin is about 320 mg. In certain embodiments, the total daily dose of aspirin is about 324 mg.

In one embodiment, pharmaceutical compositions provided herein comprise aspirin that is in a extended release form. In another embodiment, pharmaceutical compositions provided herein comprise aspirin that is in an immediate release form that is released at multiple times throughout the day.

Aspiring Release Profile

In one embodiment, provided are pharmaceutical compositions comprising niacin and aspirin wherein the total daily dose of aspirin is released from the composition over a period of time of about 2 to about 16 hours.

In one embodiment, provided are pharmaceutical compositions comprising niacin and aspirin wherein the total daily dose of aspirin is released from the composition based on aspirin release profile, wherein 80% of aspirin is released over a period of about 2 to about 16 hours following administration of the composition. In another embodiment, the total daily dose of aspirin is released from the composition based on aspirin release profile, wherein 90% of aspirin AUC is released over a period of about 2 to about 16 hours following administration of the composition. In another embodiment, the total daily dose of aspirin is released from the composition based on an aspirin release profile, wherein aspirin concentration in plasma is greater than 5% of Cmax over a period of time of about 2 to about 16 hours following administration of the composition. In another embodiment, the total daily dose of aspirin is released from the composition based on an aspirin release profile, wherein aspirin concentration in plasma is greater than 10% of Cmax over a period of time of about 2 to about 16 hours following administration of the composition.

In one embodiment, the total daily dose of aspirin is released over about 2 to about 8 hours. In another embodiment, the total daily dose of aspirin is released over about 2 to about 6 hours. In yet another embodiment, the total daily dose of aspirin is released over about 3 to about 4 hours.

In one embodiment, the total daily dose of aspirin is released from the composition based on an aspirin release profile, wherein 80% of aspirin is released over a period of about 2 to about 8 hours following administration of the composition. In another embodiment, the total daily dose of aspirin is released from the composition based on aspirin release profile, wherein 90% of aspirin is released over a period of about 2 to about 8 hours following administration of the composition. In another embodiment, the total daily dose of aspirin is released from the composition based on aspirin release profile, wherein aspirin concentration in plasma is greater than 5% of Cmax over a period of time of about 2 to about 8 hours following administration of the composition. In another embodiment, the total daily dose of aspirin is released from the composition based on aspirin release profile, wherein aspirin concentration in plasma is greater than 10% of Cmax over a period of time of about 2 to about 8 hours following administration of the composition. In still another embodiment, the total daily dose of aspirin is released from the composition based on aspirin release profile, wherein 80% of aspirin is released over a period of about 2 to about 6 hours following administration of the composition. In another embodiment, the total daily dose of aspirin is released from the composition based on aspirin release profile, wherein 90% of aspirin is released over a period of about 2 to about 6 hours following administration of the composition. In another embodiment, the total daily dose of aspirin is released from the composition based on aspirin release profile, wherein aspirin concentration in plasma is greater than 5% of Cmax over a period of time of about 2 to about 6 hours following administration of the composition. In another embodiment, the total daily dose of aspirin is released from the composition based on aspirin release profile, wherein aspirin concentration in plasma is greater than 10% of Cmax over a period of time of about 2 to about 6 hours following administration of the composition. In still another embodiment, the total daily dose of aspirin is released from the composition based on aspirin release profile, wherein 80% of aspirin is released over a period of about 3 to about 4 hours following administration of the composition. In another embodiment, the total daily dose of aspirin is released from the composition based on aspirin release profile, wherein 90% of aspirin is released over a period of about 3 to about 4 hours following administration of the composition. In another embodiment, the total daily dose of aspirin is released from the composition based on aspirin release profile, wherein aspirin concentration in plasma is greater than 10% of Cmax over a period of time of about 3 to about 4 hours following administration of the composition. In another embodiment, the total daily dose of aspirin is released from the composition based on aspirin release profile, wherein aspirin concentration in plasma is greater than 20% of Cmax over a period of time of about 3 to about 4 hours following administration of the composition.

In one embodiment, the total daily dose of aspirin is released over about 2 to about 10 hours. In another embodiment, the total daily dose of aspirin is released over about 3 to about 8 hours. In yet another embodiment, the total daily dose of aspirin is released over about 4 to about 6 hours.

In one embodiment, the total daily dose of aspirin is released from the composition based on aspirin release profile, wherein 80% of aspirin is released over a period of about 2 to about 10 hours following administration of the composition. In another embodiment, the total daily dose of aspirin is released from the composition based on aspirin release profile, wherein 90% of aspirin is released over a period of about 2 to about 10 hours following administration of the composition. In another embodiment, the total daily dose of aspirin is released from the composition based on aspirin release profile, wherein aspirin concentration in plasma is greater than 5% of Cmax over a period of time of about 2 to about 10 hours following administration of the composition. In another embodiment, the total daily dose of aspirin is released from the composition based on aspirin release profile, wherein aspirin concentration in plasma is greater than 10% of Cmax over a period of time of about 2 to about 10 hours following administration of the composition. In still another embodiment, the total daily dose of aspirin is released from the composition based on aspirin release profile, wherein 80% of aspirin is released over a period of about 3 to about 8 hours following administration of the composition. In another embodiment, the total daily dose of aspirin is released from the composition based on aspirin release profile, wherein 90% of aspirin is released over a period of about 3 to about 8 hours following administration of the composition. In another embodiment, the total daily dose of aspirin is released from the composition based on aspirin release profile, wherein aspirin concentration in plasma is greater than 5% of Cmax over a period of time of about 3 to about 8 hours following administration of the composition. In another embodiment, the total daily dose of aspirin is released from the composition based on aspirin release profile, wherein aspirin concentration in plasma is greater than 10% of Cmax over a period of time of about 3 to about 8 hours following administration of the composition. In still another embodiment, the total daily dose of aspirin is released from the composition based on aspirin release profile, wherein 80% of aspirin is released over a period of about 4 to about 6 hours following administration of the composition. In another embodiment, the total daily dose of aspirin is released from the composition based on aspirin release profile, wherein 90% of aspirin is released over a period of about 4 to about 6 hours following administration of the composition. In another embodiment, the total daily dose of aspirin is released from the composition based on aspirin release profile, wherein aspirin concentration in plasma is greater than 5% of Cmax over a period of time of about 4 to about 6 hours following administration of the composition. In another embodiment, the total daily dose of aspirin is released from the composition based on aspirin release profile, wherein aspirin concentration in plasma is greater than 10% of Cmax over a period of time of about 4 to about 6 hours following administration of the composition.

In one embodiment, the total daily dose of aspirin is released over about 3 to about 12 hours. In another embodiment, the total daily dose of aspirin is released over about 4 to about 8 hours. In yet another embodiment, the total daily dose of aspirin is released over about 3 to about 6 hours. In another embodiment, the total daily dose of aspirin is released over about 4 to about 5 hours.

In one embodiment, the total daily dose of aspirin is released from the composition based on aspirin release profile, wherein 80% of aspirin is released over a period of about 3 to about 12 hours following administration of the composition. In another embodiment, the total daily dose of aspirin is released from the composition based on aspirin release profile, wherein 90% of aspirin is released over a period of about 3 to about 12 hours following administration of the composition. In another embodiment, the total daily dose of aspirin is released from the composition based on aspirin release profile, wherein aspirin concentration in plasma is greater than 5% of Cmax over a period of time of about 3 to about 12 hours following administration of the composition. In another embodiment, the total daily dose of aspirin is released from the composition based on aspirin release profile, wherein aspirin concentration in plasma is greater than 10% of Cmax over a period of time of about 3 to about 12 hours following administration of the composition. In still another embodiment, the total daily dose of aspirin is released from the composition based on aspirin release profile, wherein 80% of aspirin is released over a period of about 4 to about 8 hours following administration of the composition. In another embodiment, the total daily dose of aspirin is released from the composition based on aspirin release profile, wherein 90% of aspirin is released over a period of about 4 to about 8 hours following administration of the composition. In another embodiment, the total daily dose of aspirin is released from the composition based on aspirin release profile, wherein aspirin concentration in plasma is greater than 5% of Cmax over a period of time of about 4 to about 8 hours following administration of the composition. In another embodiment, the total daily dose of aspirin is released from the composition based on aspirin release profile, wherein aspirin concentration in plasma is greater than 10% of Cmax over a period of time of about 4 to about 8 hours following administration of the composition. In still another embodiment, the total daily dose of aspirin is released from the composition based on aspirin release profile, wherein 80% of aspirin is released over a period of about 3 to about 6 hours following administration of the composition. In another embodiment, the total daily dose of aspirin is released from the composition based on aspirin release profile, wherein 90% of aspirin is released over a period of about 3 to about 6 hours following administration of the composition. In another embodiment, the total daily dose of aspirin is released from the composition based on aspirin release profile, wherein aspirin concentration in plasma is greater than 5% of Cmax over a period of time of about 3 to about 6 hours following administration of the composition. In another embodiment, the total daily dose of aspirin is released from the composition based on aspirin release profile, wherein aspirin concentration in plasma is greater than 10% of Cmax over a period of time of about 3 to about 6 hours following administration of the composition. In yet another embodiment, the total daily dose of aspirin is released from the composition based on aspirin release profile, wherein 80% of aspirin is released over a period of about 4 to about 5 hours following administration of the composition. In another embodiment, the total daily dose of aspirin is released from the composition based on aspirin release profile, wherein 90% of aspirin is released over a period of about 4 to about 5 hours following administration of the composition. In another embodiment, the total daily dose of aspirin is released from the composition based on aspirin release profile, wherein aspirin concentration in plasma is greater than 10% of Cmax over a period of time of about 4 to about 5 hours following administration of the composition. In another embodiment, the total daily dose of aspirin is released from the composition based on aspirin release profile, wherein aspirin concentration in plasma is greater than 20% of Cmax over a period of time of about 4 to about 5 hours following administration of the composition.

In one embodiment, the total daily dose of aspirin is released over about 4 to about 16 hours. In another embodiment, the total daily dose of aspirin is released over about 5 to about 12 hours. In yet another embodiment, the total daily dose of aspirin is released over about 6 to about 10 hours. In another embodiment, the total daily dose of aspirin is released over about 6 to about 7 hours. In another embodiment, the total daily dose of aspirin is released over about 9 to about 10 hours.

In one embodiment, the total daily dose of aspirin A is released from the composition based on aspirin release profile, wherein 80% of aspirin is released over a period of about 4 to about 16 hours following administration of the composition. In another embodiment, the total daily dose of aspirin is released from the composition based on aspirin release profile, wherein 90% of aspirin is released over a period of about 4 to about 16 hours following administration of the composition. In another embodiment, the total daily dose of aspirin is released from the composition based on aspirin release profile, wherein aspirin concentration in plasma is greater than 5% of Cmax over a period of time of about 4 to about 16 hours following administration of the composition In another embodiment, the total daily dose of aspirin is released from the composition based on aspirin release profile, wherein aspirin concentration in plasma is greater than 10% of Cmax over a period of time of about 4 to about 16 hours following administration of the composition. In still another embodiment, the total daily dose of aspirin is released from the composition based on aspirin release profile, wherein 80% of aspirin is released over a period of about 5 to about 12 hours following administration of the composition. In another embodiment, the total daily dose of aspirin is released from the composition based on aspirin release profile, wherein 90% of aspirin is released over a period of about 5 to about 12 hours following administration of the composition. In another embodiment, the total daily dose of aspirin is released from the composition based on aspirin release profile, wherein aspirin concentration in plasma is greater than 5% of Cmax over a period of time of about 5 to about 12 hours following administration of the composition. In another embodiment, the total daily dose of aspirin is released from the composition based on aspirin release profile, wherein aspirin concentration in plasma is greater than 10% of Cmax over a period of time of about 5 to about 12 hours following administration of the composition. In still another embodiment, the total daily dose of aspirin is released from the composition based on aspirin release profile, wherein 80% of aspirin is released over a period of about 6 to about 10 hours following administration of the composition. In another embodiment, the total daily dose of aspirin is released from the composition based on aspirin release profile, wherein 90% of aspirin is released over a period of about 6 to about 10 hours following administration of the composition. In another embodiment, the total daily dose of aspirin is released from the composition based on aspirin release profile, wherein aspirin concentration in plasma is greater than 5% of Cmax over a period of time of about 6 to about 10 hours following administration of the composition. In another embodiment, the total daily dose of aspirin is released from the composition based on aspirin release profile, wherein aspirin concentration in plasma is greater than 10% of Cmax over a period of time of about 6 to about 10 hours following administration of the composition. In yet another embodiment, the total daily dose of aspirin is released from the composition based on aspirin release profile, wherein 80% of aspirin is released over a period of about 6 to about 7 hours following administration of the composition. In another embodiment, the total daily dose of aspirin is released from the composition based on aspirin release profile, wherein 90% of aspirin is released over a period of about 6 to about 7 hours following administration of the composition. In another embodiment, the total daily dose of aspirin is released from the composition based on aspirin release profile, wherein aspirin concentration in plasma is greater than 5% of Cmax over a period of time of about 6 to about 7 hours following administration of the composition. In another embodiment, the total daily dose of aspirin is released from the composition based on aspirin release profile, wherein aspirin concentration in plasma is greater than 10% of Cmax over a period of time of about 6 to about 7 hours following administration of the composition. In yet another embodiment, the total daily dose of aspirin is released from the composition based on aspirin release profile, wherein 80% of aspirin is released over a period of about 9 to about 10 hours following administration of the composition. In another embodiment, the total daily dose of aspirin is released from the composition based on aspirin release profile, wherein 90% of aspirin is released over a period of about 9 to about 10 hours following administration of the composition. In another embodiment, the total daily dose of aspirin A is released from the composition based on aspirin release profile, wherein aspirin concentration in plasma is greater than 5% of Cmax over a period of time of about 9 to about 10 hours following administration of the composition. In another embodiment, the total daily dose of aspirin is released from the composition based on aspirin release profile, wherein aspirin concentration in plasma is greater than 10% of Cmax over a period of time of about 9 to about 10 hours following administration of the composition.

Niacin Release Profile

In one embodiment, provided is a modified release nicotinic acid formulation with a lag phase before niacin delivery suitable for oral administration once or twice a day dosing for treating hyperlipidemia without causing drug-induced hepatotoxicity to a level which would require said nicotinic acid formulation to be discontinued, said modified release nicotinic acid formulation exhibiting a release pattern characterized by two phases when a convoluted plasma curve for nicotinic acid released from the said modified release nicotinic acid formulation is deconvoluted using the Wagner-Nelson method, a lag phase and an extended release phase. In another embodiment, the lag phase is characterized by: i) less than 10% of the nicotinic acid dose administered is absorbed between about 2 and about 4 hours following ingestion. In another embodiment, the extended release phase being characterized by: ii) more than about 20% but less than 78% of the nicotinic acid administered being absorbed between about 7 and 8 hours following ingestion. In another embodiment, less than 90% of the nicotinic acid administered being absorbed by 9 hours following ingestion. In a yet other embodiment, a modified release nicotinic acid formulation as above is comprised of a modified release nicotinic acid formulation exhibiting a release pattern characterized by two phases, a lag phase and an extended release phase. In one embodiment, the lag phase is characterized by: i) plasma levels below 20% of the C_{MAX} for at least 3 hours after the time of ingestion and up to 16 hours following ingestion. In another embodiment, the extended release phase being characterized by: ii) plasma levels following the lag phase being maintained above 20% of the C_{MAX} for a period of at least 3 hours but less than 8 hours. In another embodiment, plasma levels following the extended release phase being less than 10% of the C_{MAX} by hour 24.

In one embodiment, a modified release nicotinic acid formulation as above, displayes the nicotinic acid absorption mean for the two phases as follows: between 1% and 10% of the nicotinic acid dose administered is absorbed during the lag phase of between ingestion and 3 and to 8 hours following ingestion. In another embodiment, less than 90% of the nicotinic acid dose administered is absorbed at about 7.5 hours following ingestion.

In one embodiment, provided are modified release nicotinic acid formulations wherein said modified release nicotinic acid formulation exhibits a release pattern characterized by two phases, a lag phase and an extended release phase. In one embodiment, the lag phase is characterized by plasma levels below 20% of the C_{MAX} for at least 3 hours after the time of ingestion and up to 16 hours following ingestion. In another embodiment, the extended release phase is characterized by the T_{MAX} of at least 6 hours but less than 20 hours following ingestion.

Aspirin Pretreatment Schedule

In one embodiment, aspirin administration comprises about 1 to about 7 days of aspirin pretreatment before initiation of niacin therapy.

In one embodiment, a subject is predosed on the day of niacin therapy with an aspirin regimen initiated about 2 to about 16 hours before niacin therapy. In another embodiment, a subject is predosed on the day of niacin therapy with an aspirin regimen initiated about 1 to about 12 hours before niacin therapy. In another embodiment, a subject is predosed on the day of niacin therapy with an aspirin regimen initiated about 2 to about 10 hours before niacin therapy. In yet another embodiment, a subject is predosed on the day of niacin therapy with an aspirin regimen initiated about 4 to about 8 hours before niacin therapy.

In one embodiment, a subject is predosed on the day of niacin therapy with aspirin regimen initiated about 16 hours before niacin therapy. In another embodiment, a subject is predosed on the day of niacin therapy with aspirin regimen initiated about 14 hours before niacin therapy. In another embodiment, a subject is predosed on the day of niacin therapy with aspirin regimen initiated about 12 hours before niacin therapy. In another embodiment, a subject is predosed on the day of niacin therapy with aspirin regimen initiated about 10 hours before niacin therapy. In one embodiment, a subject is predosed on the day of niacin therapy with aspirin regimen initiated about 8 hours before niacin therapy. In one embodiment, a subject is predosed on the day of niacin therapy with aspirin regimen initiated about 6.5 hours before niacin therapy. In yet another embodiment, a subject is predosed on the day of niacin therapy with aspirin regimen initiated about 6 hours before niacin therapy. In another embodiment, a subject is predosed on the day of niacin therapy with aspirin regimen initiated about 5 hours before niacin therapy. In another embodiment, a subject is predosed on the day of niacin therapy with aspirin regimen initiated about 4 hours before niacin therapy In another embodiment, a subject is predosed on the day of niacin therapy with aspirin regimen initiated about 3 hours before niacin therapy. In another embodiment, a subject is predosed on the day of niacin therapy with aspirin regimen initiated about 2 hours before niacin therapy. In another embodiment, a subject is predosed on the day of niacin therapy with aspirin regimen initiated about 1 hour before niacin therapy.

In one embodiment, a subject is predosed on the day of niacin therapy with aspirin regimen, wherein about 80% of niacin AUC is not released until after about 16 hours of predosing with aspirin. In another embodiment, about 90% of niacin AUC is not released until after about 16 hours of predosing with aspirin. In another embodiment, the plasma concentration of niacin is less than 20% of Cmax until after about 16 hours of predosing with aspirin. In yet another embodiment, the plasma concentration of niacin is less than 10% of Cmax until after about 16 hours of predosing with aspirin.

In one embodiment, a subject is predosed on the day of niacin therapy with aspirin regimen, wherein about 80% of niacin AUC is not released until after about 14 hours of predosing with aspirin. In another embodiment, about 90% of niacin AUC is not released until after about 14 hours of predosing with aspirin. In another embodiment, the plasma concentration of niacin is less than 20% of Cmax until after about 14 hours of predosing with aspirin. In yet another embodiment, the plasma concentration of niacin is less than 10% of Cmax until after about 14 hours of predosing with aspirin.

In one embodiment, a subject is predosed on the day of niacin therapy with aspirin regimen, wherein about 80% of niacin AUC is not released until after about 12 hours of predosing with aspirin. In another embodiment, about 90% of niacin AUC is not released until after about 12 hours of predosing with aspirin. In another embodiment, the plasma concentration of niacin is less than 20% of Cmax until after about 12 hours of predosing with aspirin. In yet another embodiment, the plasma concentration of niacin is less than 10% of Cmax until after about 12 hours of predosing with aspirin.

In one embodiment, a subject is predosed on the day of niacin therapy with aspirin regimen, wherein about 80% of niacin AUC is not released until after about 10 hours of predosing with aspirin. In another embodiment, about 90% of niacin AUC is not released until after about 10 hours of predosing with aspirin. In another embodiment, the plasma concentration of niacin is less than 20% of Cmax until after about 10 hours of predosing with aspirin. In yet another embodiment, the plasma concentration of niacin is less than 10% of Cmax until after about 10 hours of predosing with aspirin.

In one embodiment, a subject is predosed on the day of niacin therapy with aspirin regimen, wherein about 80% of niacin AUC is not released until after about 8 hours of predosing with aspirin. In another embodiment, about 90% of niacin AUC is not released until after about 8 hours of predosing with aspirin. In another embodiment, the plasma concentration of niacin is less than 20% of Cmax until after about 8 hours of predosing with aspirin. In yet another embodiment, the plasma concentration of niacin is less than 10% of Cmax until after about 8 hours of predosing with aspirin.

In one embodiment, a subject is predosed on the day of niacin therapy with aspirin regimen, wherein about 80% of niacin AUC is not released until after about 6 hours of predosing with aspirin. In another embodiment, about 90% of niacin AUC is not released until after about 6 hours of predosing with aspirin. In another embodiment, the plasma concentration of niacin is less than 20% of Cmax until after about 6 hours of predosing with aspirin. In yet another embodiment, the plasma concentration of niacin is less than 10% of Cmax until after about 6 hours of predosing with aspirin.

In one embodiment, a subject is predosed on the day of niacin therapy with aspirin regimen, wherein about 80% of niacin AUC is not released until after about 4 hours of predosing with aspirin. In another embodiment, about 90% of niacin AUC is not released until after about 4 hours of predosing with aspirin. In another embodiment, the plasma concentration of niacin is less than 20% of Cmax until after about 4 hours of predosing with aspirin. In yet another embodiment, the plasma concentration of niacin is less than 10% of Cmax until after about 4 hours of predosing with aspirin.

In one embodiment, a subject is predosed on the day of niacin therapy with aspirin regimen, wherein about 80% of niacin AUC is not released until after about 3 hours of predosing with aspirin. In another embodiment, about 90% of niacin AUC is not released until after about 3 hours of predosing with aspirin. In another embodiment, the plasma concentration of niacin is less than 20% of Cmax until after about 3 hours of predosing with aspirin. In yet another embodiment, the plasma concentration of niacin is less than 10% of Cmax until after about 3 hours of predosing with aspirin.

In one embodiment, a subject is predosed on the day of niacin therapy with aspirin regimen, wherein about 80% of niacin AUC is not released until after about 2 hours of predosing with aspirin. In another embodiment, about 90% of niacin AUC is not released until after about 2 hours of predosing with aspirin. In another embodiment, the plasma concentration of niacin is less than 20% of Cmax until after about 2 hours of predosing with aspirin. In yet another embodiment, the plasma concentration of niacin is less than 10% of Cmax until after about 2 hours of predosing with aspirin.

In one embodiment, a subject is predosed on the day of niacin therapy with aspirin regimen, wherein about 80% of niacin AUC is not released until after about 1 hour of predosing with aspirin. In another embodiment, about 90% of niacin AUC is not released until after about 1 hour of predosing with aspirin. In another embodiment, the plasma concentration of niacin is less than 20% of Cmax until after about 1 hour of predosing with aspirin. In yet another embodiment, the plasma concentration of niacin is less than 10% of Cmax until after about 1 hour of predosing with aspirin.

In one embodiment, pharmaceutical compositions provided herein comprise aspirin that is in a extended release form. In another embodiment, pharmaceutical compositions provided herein comprise aspirin that is in an immediate release form that is released at multiple times throughout the day. In another embodiment, pharmaceutical compositions provided herein comprise niacin that is in an extended release form. In another embodiment, pharmaceutical compositions provided herein comprise niacin that is in an immediate release form.

Aspirin Post Treatment Schedule

In one embodiment, provided are pharmaceutical compositions and formulations for continued administration of aspirin while niacin is being administered. In another embodiment, a pill, capsule or other delivery source can be formulated to contain an amount of aspirin to be released before and after niacin administration.

In one embodiment, a pill can be formulated to contain an amount of aspirin to be released about 6.5 hours before and about 0.5 hour to about 3.5 hours after initiation of niacin therapy. In another embodiment, a pill, capsule or other delivery source can be formulated to contain an amount of aspirin to be released about 6 hours before and about 5 hours after initiation of niacin therapy. In another embodiment, a pill can be formulated to contain an amount of aspirin to be released about 5 hours before and about 5 hour after initiation of niacin therapy. In another embodiment, a pill can be formulated to contain an amount of aspirin to be released about 5 hours before and about 1 hour to about 5 hours after initiation of niacin therapy. In another embodiment, a pill can be formulated to contain an amount of aspirin to be released about 4 hours before and about 1 hour to about 6 hours after initiation of niacin therapy. In another embodiment, a pill can be formulated to contain an amount of aspirin to be released about 4 hours before and about 4 hours after initiation of niacin therapy. In another embodiment, a pill can be formulated to contain an amount of aspirin to be released about 4 hours before and about 3 hours after initiation of niacin therapy. In another embodiment, a pill can be formulated to contain an amount of aspirin to be released about 3 hours before and about 3 hours after initiation of niacin therapy. In another embodiment, a pill can be formulated to contain an amount of aspirin to be released about 3 hours before and about 2 hours after initiation of niacin therapy. In yet another embodiment, a pill can be formulated to contain an amount of aspirin to be released about 3 hours before and about 1 hour after initiation of niacin therapy.

In another embodiment, a pill, capsule or other delivery source can be formulated to contain an amount of aspirin to be released as multiple small doses.

In one embodiment, pharmaceutical compositions provided herein comprise aspirin that is in a extended release form. In another embodiment, pharmaceutical compositions provided herein comprise aspirin that is in an immediate release form that is administered at multiple times throughout the day in small increments or pulsatile way.

Combination Therapy

In one embodiment, provided are pharmaceutical compositions comprising niacin and aspirin that reduce niacin-induced flushing, further comprising a lipid-lowering drug other than niacin.

In one embodiment, the lipid lowering drugs are HMGCoA reductase inhibitors (statins). The statins comprised by the pharmaceutical compositions include, but are not limited to, lovastatin, simvastatin, pravastatin, fluvastatin, cerivastatin, rosuvastatin, and atorvastatin. In certain embodiments, the statin is atorvastatin.

In another embodiment, the lipid-lowering drugs are fibrates, bile acid sequestrants, cholesterol absorption inhibitors and other agents as defined under anti-inflammatory drugs or NSAIDS.

In another embodiment, provided are niacin and aspirin formulations further comprising a lipid lowering drug other than niacin to treat atherosclerosis, cardiovascular diseases, dyslipidemias, cancer, Alzheimer disease, and metabolic diseases, including, but not limited to, diabetes, obesity, metabolic syndrome, and genetic abnormalities associated with increased cardiovascular risk.

In another embodiment, provided are niacin and aspirin formulations further comprising anti-obesity agents, anti-diabetic agents and anti-hypertensive agents.

Microparticle Formulations

In one embodiment, provided are aspirin 80-81 mg capsules containing microparticles having different dissolution profiles and characteristics. In a specific embodiment, provided are aspirin 81 mg capsules containing microparticles having different dissolution profiles and characteristics. In another embodiment, provided are aspirin 160-162 mg capsules containing microparticles having different dissolution profiles and characteristics. In another embodiment, provided are aspirin 240-243 mg capsules containing microparticles having different dissolution profiles and characteristics. In another embodiment, provided are aspirin 320-325 mg capsules containing microparticles having different dissolution profiles and characteristics.

In another embodiment, aspirin microparticles may be formulated by methods described in U.S. Pat. No. 6,022,562. In another embodiment, the aspirin microparticles are defined by a dissolution profile, wherein 80% of aspirin is released during 4-5 hours following administration of the capsule (A1 microparticles). In another embodiment, the aspirin microparticles are defined by a dissolution profile, wherein 80% of aspirin is released during 6-7 hours following administration of the capsule (A2 microparticles). In yet another embodiment, the aspirin microparticles are defined by a dissolution profile, wherein 80% of aspirin is released during 9-10 hours following administration of the capsule (A3 microparticles).

In one embodiment, A1, A2 and A3 microparticles have been manufactured with different coating ratios of the same coating composition. In one embodiment, the coating composition encompasses ethylcellulose or the like, the methacrylic acid copolymer type B or the like, the methacrylic acid copolymer type C or the like, castor oil or the like, hydrogenated cottonseed oil or the like, povidone or the like, tartaric acid or the like, and magnesium stearate or the like. Further guidance for equivalent ingredients that could be used can be found in Remington's Pharmaceutical Sciences, 18^{th} Edition, Gennaro et al., eds., Mack Printing Company, Easton, Pa., 1990.

In one embodiment, aspirin microparticle coating ratio is about 2.5% to about 15%. In another embodiment, the amounts of the constituents are the following: acetylsalicylic acid 80%-98%, ethylcellulose 1%-10%, castor oil 0.01%-1.5%, povidone 0.05%-1%, tartaric acid 0%-1%, and magnesium stearate 0%-2%. The coating ratios have been adjusted for each of the microparticles to meet the specific dissolution target profile. The coating composition is not pH sensitive, and the dissolution is not affected by the location in the gastro-intestinal tract.

In one embodiment, provided are niacin capsules comprising 250 mg of niacin microparticles, having different dissolution profiles and characteristics. In one embodiment, provided are niacin capsules comprising 500 mg of niacin microparticles, having different dissolution profiles and characteristics.

In one embodiment, the niacin microparticles may be formulated by methods described in WO 07/036671. In one embodiment, niacin microparticles are characterized by a slow release of niacin at pH 1-3 with less than 10% release at 5 hours following administration of the microparticles. In another embodiment, niacin microparticles are characterized by a slow release of niacin at pH 1-3 with less than 20% release at 5 hours following administration of the microparticles. In another embodiment, niacin microparticles are characterized by a slow release of niacin at pH 1-3 with less than 10% release at 4 hours following administration of the microparticles. In another embodiment, niacin microparticles are characterized by a slow release of niacin at pH 1-3 with less than 20% release at 4 hours following administration of the microparticles. In another embodiment, niacin microparticles are characterized by a slow release of niacin at pH 1-3 with less than 10% release at 3 hours following administration of the microparticles. In another embodiment, niacin microparticles are characterized by a slow release of niacin at pH 1-3 with less than 20% release at 3 hours following administration of the microparticles.

In another embodiment, niacin microparticles are characterized by about 80% release of niacin at pH that is higher that the triggered pH. In another embodiment, the triggered pH is in the range of about 5.5 to about 8.0. In another embodiment, the triggered pH is about 6.0. In another embodiment, the triggered pH is about 6.5. In yet another embodiment, the triggered pH is about 7. In yet another embodiment, the triggered pH is about 7.5.

In one embodiment, the niacin microparticles are characterized by about a 5 h lag time following administration of the microparticles, with the following release activated at a pH of about 6 (N1 microparticles). In another embodiment, the niacin microparticles are characterized by an about 5 h lag time following administration of the microparticles, with the following release activated at a pH of about 6.5 (N2 microparticles). In yet another embodiment, the niacin microparticles are characterized by an about 5 h lag time following administration of the microparticles, with the following release activated at a pH of about 7 (N3 microparticles).

In one embodiment, N1, N2 and N3 microparticles have been obtained by coating niacin particles with a composition suitable for safe passage through the stomach after swallowing, then allowing release in different gastro-intestinal tract (GIT) segments. In one embodiment, the coating composition comprises two hydrophilic metacrylic polymers with different pH dependent solubility's and one hydrophobic material. In another embodiment, the coating composition encompasses ethylcellulose or the like, the methacrylic acid copolymer type B or the like, the methacrylic acid copolymer type C or the like, castor oil or the like, hydrogenated cottonseed oil or the like, povidone or the like, tartaric acid or the like, and magnesium stearate or the like. Further guidance for equivalent ingredients that could be used can be found in Remington's Pharmaceutical Sciences, 18^{th} Edition, Gennaro et al., eds., Mack Printing Company, Easton, Pa., 1990.

In one embodiment, niacin microparticle coating ratio is about 10% to about 30%. In another embodiment, the amounts of the constituents are the following: nicotinic acid 60%-90%, methacrylic acid copolymer type C (L100-55) 0%-15%, methacrylic acid copolymer type B (S100) 0%-15%, and cottonseed oil 2%-15%.

In one embodiment, provided is a formulation comprising niacin microparticles having a reduced capacity to provoke a flushing reaction in a subject, wherein the niacin microparticles having a specific release profile for niacin, and a non-steroidal anti-inflammatory drug microparticles (NSAID), wherein the NSAID is a member of the group consisting of aspirin, ibuprofen, indomethacin, phenylbutazone and naproxen, whereas the NSAID is present in an amount effective to reduce a cutaneous flushing caused by the niacin, which are the amounts shown for that member: ibuprofen—about 500 mg; indomethacin—30 mg; phenylbutazone—about 300 mg; naproxen—about 300 mg; aspirin—about 500 mg; and wherein the NSAID microparticles having a specific release profile for the NSAID. In one embodiment, the amount of aspirin is about 80 to about 1000 mg.

In one embodiment, provided is a combination formulation comprised of microparticles of aspirin that prevent or reduce aspirin hydrolysis prior to the aspirin's release from the formulation.

In one embodiment, aspirin microparticles are mixed with niacin microparticles to obtain a formulation comprising two types of microparticles with different release profiles. In another embodiment, provided is a combination formulation comprising a first population of microparticles and a second population of microparticles, wherein the first population of microparticles is an aspirin formulation having a first release profile, and wherein the second population of microparticles is niacin formulation having a second release profile, wherein the first population of microparticles and the second population of microparticles are mixed. In yet another embodiment one or more populations of aspirin microparticles having different release profiles can be mixted with one or more populations of niacin microparticles.

In one embodiment, provided is a combination formulation wherein pH sensitive microparticles are used to control release of niacin. In one embodiment, aspirin is released in a pH independent fashion. In another embodiment, niacin is released in a pH dependent fashion, wherein the pH for release is about 5.5 to about 8.0, in certain embodiments, about 5.5, 6.0, 6.5, 7.0, or 7.5.

In one embodiment, provided is a combination formulation comprising a mixture of aspirin microparticles and niacin microparticles so that aspirin and niacin are kept physically separated. In one embodiment, the aspirin microparticles and the niacin microparticles are administered at the same time as one formulation, having a lag time between release of the drugs. In one embodiment, the combination formulation is administered at bedtime. In yet another embodiment the combination formulation is administered in the evening, for example, from about 4:30 pm to about 2:00 am with or without food.

In one embodiment, provided is a combination formulation comprising a mixture of aspirin microparticles and niacin microparticles so that aspirin and niacin are kept physically separated. In one embodiment, the aspirin microparticles and the niacin microparticles are administered at the same time as one formulation, having a time lag between release of the drugs. In one embodiment, the formulation is administered in the evening, with or without food.

In one embodiment, provided is a combination formulation, wherein one capsule is used to orally pretreat a patient with aspirin and then provide a patient with a niacin dose. In one embodiment, one capsule comprises microparticles of aspirin and microparticles of niacin having different dissolution profiles. Due to niacin lag time, aspirin is released earlier, then niacin is released, for example, 4-10 hours following administration of the formulation. In one embodiment, aspirin released about 4-5 hours following administration of the formulation. In another embodiment, aspirin released about 6-7 hours following administration of the formulation. In yet another embodiment, aspirin released about 9-10 hours following administration of the formulation.

In one embodiment, provided is a combination formulation, wherein aspirin and niacin are given to a patient as one daily dose. A daily dose may contain one or multiple pills, for example 2, 3, 4, or more pills. In yet another embodiment, the combination formulation is given to the patient in two daily doses.

In one embodiment, provided is a combination formulation comprising aspirin microparticles and niacin microparticles, wherein the dissolution profiles are similar within the aspirin microparticles and within the niacin microparticles regardless of aspirin dose and niacin dose.

In one embodiment, provided is a combination formulation comprising aspirin microparticles and niacin microparticles having a inter- and intra-subject variations in terms of PK/PD and safety. In one embodiment, distribution of multiple microparticles within the capsules causes decreased product variability between patients.

In one embodiment, provided is a formulation or pharmaceutical composition comprising aspirin microparticles and niacin microparticles in a suspension to avoid an undesirable taste or after taste. In one embodiment, provided is an effervescent formulation to avoid an undesirable taste or after taste.

In one embodiment, provided is a formulation or pharmaceutical composition comprising coated aspirin microparticles and coated niacin microparticles that are tasteless.

In one embodiment, provided are multiple dosing units comprising a combination of aspirin microparticles and niacin microparticles.

In yet another embodiment, provided is an oral dosage form comprising a combination of extended release aspirin microparticles and pH-dependent release niacin microparticles in combination with an immediate release statin. In one embodiment, the statin is atorvastatin in a daily dose of about 2.5 to 80 mg.

In yet another embodiment, provided is a oral dosage form comprising a combination of extended release aspirin microparticles and pH-dependent release niacin microparticles in combination with an immediate or extended release fibrate. In one embodiment, the fibrate is fenofibrate in a daily dose of about 35 to 400 mg.

In yet another embodiment, provided is a oral dosage form comprising a combination of extended release aspirin microparticles and pH-dependent release niacin microparticles in combination with an immediate release cholesterol absorption inhibitor. In one embodiment, the cholesterol absorption inhibitor is ezetimibe in a daily dose of about 2.5 to 10 mg.

Pharmaceutical Compositions

The formulations disclosed in the U.S. Pat. No. 5,981,555 are effective to reduce niacin flushing by using subsequent or concomitant administration of formulations containing homogenous mixtures of active principles. Provided herein are extended release pharmaceutical compositions and formulations of aspirin and niacin with or without other active principles including, but not limited to, statins, fibrates, cholesterol absorption inhibitors, TZDs, PPAR agonists, PGD2 receptor inhibitors, P2Y13 ligands, CETP inhibitors and PCSK9 ligands, which provide differentiated release of aspirin and niacin. An extended release formulation can be characterized by the release of drug from the dosage form being retarded such that less than 80% of the drug is released after about one hour or less than 80% of the drug is released after about 2 hours.

Another key feature of an extended release formulation is that the C_{MAX} of the drug is lower than a dosage form where the release rate is not retarded, although there may be an associated reduction in AUC, this reduction will typically be less than the proportional reduction of the C_{MAX} however. The T_{MAX} occurs at a later time than would be true for a dosage form where the release rate is not retarded although this is not always true, particularly if there is an element of immediate release in the dosage form.

In one embodiment, an aspirin extended release formulation is where the aspirin plasma levels are sustained above 5% -10% or 10%-20% of the C_{MAX} for a period of longer than about two to three hours or the T_{MAX} occurs at more than one hour following ingestion. In another embodiment, a niacin extended release formulation is one where the plasma levels are maintained above 5%-10% or 10-20% of the C_{MAX} for a period of longer than 3 hours or the T_{MAX} occurs later than two hours.

In one embodiment, pharmaceutical compositions provided herein utilize aspirin and nicotinic acid. In another embodiment, pharmaceutical compositions provided herein utilize aspirin and nicotinic acid metabolites.

In one embodiment, pharmaceutical compositions and formulations described herein contain microparticles of the drug substance each individually coated by a film-forming mixture of excipients whose composition predetermines the targeted dissolution properties of aspirin and niacin as a function of pH. In another embodiment, a extended release aspirin is combined with a delayed and/or extended release niacin with an aspirin daily dose of about 80 mg to about 400 mg and a niacin daily dose of about 250 to about 2000 mg. In certain embodiments, the niacin components have neglible release profile at acidic pH with a pH-controlled delay in a neutral or basic pH range that allows the passage through the stomach and subsequent release in a targeted part of the intestine corresponding to the pH triggered by the combination formulation. See, e.g., Read, N. W. et al. Gut (1986), 27, p.300-308. (FIG. 1). In other embodiments, without being bound by any theory, theoretical calculations of in vivo absorption by the method described in Read (supra) show that variation of excipients in the coating of niacin particles could induce a time-dependent dissolution in the intestinal tract. In certain embodiments, aspirin formulated as such is more stable by avoiding hydrolysis and fast metabolization.

In one embodiment, the timing and the rate of the release of aspirin and niacin are managed and coordinated in order to reduce flushing but without causing liver toxicity. In one embodiment, the timing and rate of release are managed such that aspirin is provided at a sufficient amount of time before the appearance of niacin therapy in the bloodstream (initiation of niacin therapy), and for a sufficient amount of time after the appearance of niacin such that the action of aspirin is maximized and flushing is minimized. The niacin release is modified such that there is a lag phase between ingestion and initiation of release (initiation of niacin therapy) as well as an extended release phase after the initiation of release. The time between ingestion of niacin and completion of release is thus longer than acceptable forms of niacin extended release formulations that have low levels of liver toxicity and more typical of sustained release formulations of niacin that are associated with unacceptable levels of liver toxicity, however, due to the lag phase, compositions provided herein do not cause patient discontinuation of naicin therapy due to liver toxicity and are associated with a reduction in flushing.

Pharmaceutical compositions provided herein comprise niacin and an NSAID that reduce niacin-induced flushing. NSAIDs suitable for pharmaceutical compositions provided herein include, but not limited to, aspirin, ibuprofen, indomethacin, phenylbutazone and naproxen. Also prostaglandin receptor blockers, including but not limited to, laropiprant or compounds disclosed in the U.S. Patent Publication Nos. 2004/0229844 and 2005/0154044 can be employed.

In one embodiment, provided are pharmaceutical compositions comprising niacin and ibuprofen that reduce niacin-induced flushing comprising total ibuprofen daily doses of about 120-500 mg.

In another embodiment, provided are pharmaceutical compositions comprising niacin and indomethacin that reduce niacin-induced flushing comprising total indomethacin daily doses of about 25-30 mg.

In yet another embodiment, provided are pharmaceutical compositions comprising niacin and phenylbutazone that reduce niacin-induced flushing comprising total phenylbutazone daily doses of about 150-300 mg.

In another embodiment, provided are pharmaceutical compositions comprising niacin and naproxen that reduce niacin-induced flushing comprising total naproxen daily doses of about 150-300 mg.

In one embodiment, pharmaceutical compositions provided herein comprise a combination of niacin and aspirin used in a dosing regimen effective to reduce niacin-induced flushing. In one embodiment, the total aspirin daily dose is about 80 to about 2000 mg. In another embodiment, the total aspirin daily dose is about 80 to about 500 mg. In another embodiment, the total aspirin daily dose is about 80 to about 400 mg.

In one embodiment, pharmaceutical compositions provided herein comprise niacin with about 80% to about 100% bioavailability. In one embodiment, pharmaceutical compositions provided herein comprise niacin with about 80% bioavailability. In one embodiment, pharmaceutical compositions provided herein comprise niacin with about 90% bioavailability. In one embodiment, pharmaceutical compositions provided herein comprise niacin with about 100% bioavailability.

In one embodiment, the total aspirin daily dose is about 80 mg that is released over a 5 hour period. In another embodiment, the total aspirin daily dose is about 80 mg that is released over a 4 hour period. In yet another embodiment, the total aspirin daily dose is about 80 mg that is released over a 3 hour period. In another embodiment, the total aspirin daily dose is about 80 mg that is released over a 2 hour period. In another embodiment, a subject is predosed on the day of niacin therapy with a similar aspirin regimen initiated about 2 to about 16 hours before niacin therapy.

In one embodiment, the total aspirin daily dose is about 120 mg that is released over a 6 hour period. In another embodiment, the total aspirin daily dose is about 120 mg that is released over a 4 hour period. In yet another embodiment, the total aspirin daily dose is about 120 mg that is released over a 3 hour period. In another embodiment, the total aspirin daily dose is about 120 mg that is released over a 2 hour period. In another embodiment, a subject is predosed on the day of niacin therapy with a similar aspirin regimen initiated about 2 to about 16 hours before niacin therapy.

In one embodiment, the total aspirin daily dose is about 160 mg that is released over a 8 hour period. In another embodiment, the total aspirin daily dose is about 160 mg that is released over a 5-6 hour period. In yet another embodiment, the total aspirin daily dose is about 160 mg that is released over a 4 hour period. In another embodiment, the total aspirin daily dose is about 160 mg that is released over a 2-3 hour period. In another embodiment, a subject is predosed on the day of niacin therapy with a similar aspirin regimen initiated about 2 to about 16 hours before niacin therapy.

In one embodiment, the total aspirin daily dose is about 240 mg that is released over a 12 hour period. In another embodiment, the total aspirin daily dose is about 240 mg that is released over a 8 hour period. In yet another embodiment, the total aspirin daily dose is about 240 mg that is released over a 6 hour period. In another embodiment, the total aspirin daily dose is about 240 mg that is released over a 4 hour period. In another embodiment, a subject is predosed on the day of niacin therapy with a similar aspirin regimen initiated about 2 to about 16 hours before niacin therapy.

In one embodiment, the total aspirin daily dose is about 320 mg that is released over a 16 hour period. In another embodiment, the total aspirin daily dose is about 320 mg that is released over a 10-11 hour period. In yet another embodiment, the total aspirin daily dose is about 320 mg that is released over a 8 hour period. In another embodiment, the total aspirin daily dose is about 320 mg that is released over a 5-6 hour period. In another embodiment, the total aspirin daily dose is about 320 mg that is released over a 4 hour period. In another embodiment, a subject is predosed on the day of niacin therapy with a similar aspirin regimen initiated about 2 to about 16 hours before niacin therapy.

In one embodiment, the total daily dose of aspirin is about 81 mg that is released from the composition based on an aspirin release profile, wherein 80% of aspirin AUC is released over a period of about 5 hours following administration of the composition. In another embodiment, the total daily dose of aspirin is about 81 mg that is released from the composition based on an aspirin release profile, wherein 90% of aspirin AUC is released over a period of about 5 hours following administration of the composition. In another embodiment, the total daily dose of asprin is about 81 mg that is released from the composition based on an aspirin release profile, wherein aspirin concentration in plasma is greater than 10% of Cmax over a period of time of about 5 hours following administration of the composition. In yet another embodiment, the total daily dose of aspirin is about 81 mg that is released from the composition based on an aspirin release profile, wherein aspirin concentration in plasma is greater than 20% of Cmax over a period of time of about 5 hours following administration of the composition.

In one embodiment, the total daily dose of aspirin is about 81 mg that is released from the composition based on an aspirin release profile, wherein 80% of aspirin AUC is released over a period of about 4 hours following administration of the composition. In another embodiment, the total daily dose of aspirin is about 81 mg that is released from the composition based on an aspirin release profile, wherein 90% of aspirin AUC is released over a period of about 4 hours following administration of the composition. In another embodiment, the total daily dose of aspirin is about 81 mg that is released from the composition based on an aspirin release profile, wherein aspirin concentration in plasma is greater than 10% of Cmax over a period of time of about 4 hours following administration of the composition. In yet another embodiment, the total daily dose of aspirin is about 81 mg that is released from the composition based on an aspirin release profile, wherein aspirin concentration in plasma is greater than 20% of Cmax over a period of time of about 4 hours following administration of the composition.

In one embodiment, the total daily dose of aspirin is about 81 mg that is released from the composition based on an aspirin release profile, wherein 80% of aspirin AUC is released over a period of time of about 3 hours following administration of the composition. In another embodiment, the total daily dose of aspirin is about 81 mg that is released from the composition based on an aspirin release profile, wherein 90% of aspirin AUC is released over a period of about 3 hours following administration of the composition. In another embodiment, the total daily dose of aspirin is released from the composition based on an aspirin release profile, wherein aspirin concentration in plasma is greater than 10% of Cmax over a period of time of about 3 hours following administration of the composition. In yet another embodiment, the total daily dose of aspirin is released from the composition based on an aspirin release profile, wherein aspirin concentration in plasma is greater than 20% of Cmax over a period of time of about 3 hours following administration of the composition.

In one embodiment, the total daily dose of aspirin is about 81 mg that is released from the composition based on an aspirin release profile, wherein 80% of aspirin AUC is released over a period of about 2 hours following administration of the composition. In another embodiment, the total daily dose of aspirin is about 81 mg that is released from the composition based on an aspirin release profile, wherein 90% of aspirin AUC is released over a period of about 2 hours following administration of the composition. In another embodiment, the total daily dose of aspirin is released from the composition based on an aspirin release profile, wherein aspirin concentration in plasma is greater than 10% of Cmax over a period of time of about 2 hours following administration of the composition. In yet another embodiment, the total daily dose of aspirin is released from the composition based on an aspirin release profile, wherein aspirin concentration in plasma is greater than 20% of Cmax over a period of time of about 2 hours following administration of the composition.

In one embodiment, a subject is predosed on the day of niacin therapy with a total daily dose of aspirin of about 81 mg, wherein about 80% of niacin AUC is not released from the composition until about 1 hour to about 5 hours of predosing with aspirin. In another embodiment, about 90% of niacin AUC is not released from the composition until about 1 hour to about 5 hours of predosing with aspirin. In another embodiment, the plasma concentration of niacin is less than 20% of Cmax until about 1 hour to about 5 hours of predosing with the total daily dose of aspirin of about 81 mg. In yet another embodiment, the plasma concentration of niacin is less than 10% of Cmax until about 1 hour to about 5 hours of predosing with the total daily dose of aspirin of about 81 mg.

In one embodiment, the total daily dose of aspirin is about 120 mg that is released from the composition based on an aspirin release profile, wherein 80% of aspirin AUC is released over a period of about 6 hours following administration of the composition. In another embodiment, the total daily dose of aspirin is about 120 mg that is released from the composition based on an aspirin release profile, wherein 90% of aspirin AUC is released over a period of about 6 hours following administration of the composition. In another embodiment, the total daily dose of aspirin is released from the composition based on an aspirin release profile, wherein aspirin concentration in plasma is greater than 5% of Cmax over a period of time of about 6 hours following administration of the composition. In yet another embodiment, the total daily dose of aspirin is released from the composition based on an aspirin release profile, wherein aspirin concentration in plasma is greater than 10% of Cmax over a period of time of about 6 hours following administration of the composition.

In one embodiment, the total daily dose of aspirin is about 120 mg that is released from the composition based on an aspirin release profile, wherein 80% of aspirin AUC is released over a period of about 4 hours following administration of the composition. In another embodiment, the total daily dose of aspirin is about 120 mg that is released from the composition based on an aspirin release profile, wherein 90% of aspirin AUC is released over a period of about 4 hours following administration of the composition. In another embodiment, the total daily dose of aspirin is released from the composition based on an aspirin release profile, wherein aspirin concentration in plasma is greater than 10% of Cmax over a period of time of about 4 hours following administration of the composition. In yet another embodiment, the total daily dose of aspirin is released from the composition based on an aspirin release profile, wherein aspirin concentration in plasma is greater than 20% of Cmax over a period of time of about 4 hours following administration of the composition.

In one embodiment, the total daily dose of aspirin is about 120 mg that is released from the composition based on an aspirin release profile, wherein 80% of aspirin AUC is released over a period of about 3 hours following administration of the composition. In another embodiment, the total daily dose of aspirin is about 120 mg that is released from the composition based on an aspirin release profile, wherein 90% of aspirin AUC is released over a period of about 3 hours following administration of the composition. In another embodiment, the total daily dose of aspirin is released from the composition based on an aspirin release profile, wherein aspirin concentration in plasma is greater than 10% of Cmax over a period of time of about 3 hours following administration of the composition. In yet another embodiment, the total daily dose of aspirin is released from the composition based on an aspirin release profile, wherein aspirin concentration in plasma is greater than 20% of Cmax over a period of time of about 3 hours following administration of the composition.

In one embodiment, the total daily dose of aspirin is about 120 mg that is released from the composition based on an aspirin release profile, wherein 80% of aspirin AUC is released over a period of about 2 hours following administration of the composition. In another embodiment, the total daily dose of aspirin is about 120 mg that is released from the composition based on an aspirin release profile, wherein 90% of aspirin AUC is released over a period of about 2 hours following administration of the composition. In another embodiment, the total daily dose of aspirin AUC is released from the composition based on an aspirin release profile, wherein aspirin concentration in plasma is greater than 10% of Cmax over a period of time of about 2 hours following administration of the composition. In yet another embodiment, the total daily dose of aspirin is released from the composition based on an aspirin release profile, wherein aspirin concentration in plasma is greater than 20% of Cmax over a period of time of about 2 hours following administration of the composition.

In one embodiment, a subject is predosed on the day of niacin therapy with a total daily dose of aspirin of about 120 mg, wherein about 80% of niacin AUC is not released from the composition until about 2 hour to about 8 hours of predosing with aspirin. In another embodiment, about 90% of niacin AUC is not released from the composition until about 2 hour to about 8 hours of predosing with aspirin. In another embodiment, the plasma concentration of niacin is less than 20% of Cmax until about 2 hour to about 8 hours of predosing with the total daily dose of aspirin of about 120 mg. In yet another embodiment, the plasma concentration of niacin is less than 10% of Cmax until about 2 hour to about 8 hours of predosing with the total daily dose of aspirin of about 120 mg.

In one embodiment, the total daily dose of aspirin is about 160 mg that is released from the composition based on an aspirin release profile, wherein 80% of aspirin AUC is released over a period of about 8 hours following administration of the composition. In another embodiment, the total daily dose of aspirin is about 160 mg that is released from the composition based on an aspirin release profile, wherein 90% of aspirin AUC is released over a period of about 8 hours following administration of the composition. In another embodiment, the total daily dose of aspirin is released from the composition based on an aspirin release profile, wherein aspirin concentration in plasma is greater than 5% of Cmax over a period of time of about 8 hours following administration of the composition. In yet another embodiment, the total daily dose of aspirin is released from the composition based on an aspirin release profile, wherein aspirin concentration in plasma is greater than 10% of Cmax over a period of time of about 8 hours following administration of the composition.

In one embodiment, the total daily dose of aspirin is about 160 mg that is released from the composition based on an aspirin release profile, wherein 80% of aspirin AUC is released over a period of about 5-6 hours following administration of the composition. In another embodiment, the total daily dose of aspirin is about 160 mg that is released from the composition based on an aspirin release profile, wherein 90% of aspirin AUC is released over a period of about 5-6 hours following administration of the composition. In another embodiment, the total daily dose of aspirin is released from the composition based on an aspirin release profile, wherein aspirin concentration in plasma is greater than 5% of Cmax over a period of time of about 5-6 hours following administration of the composition. In yet another embodiment, the total daily dose of aspirin is released from the composition based on an aspirin release profile, wherein aspirin concentration in plasma is greater than 100% of Cmax over a period of time of about 5-6 hours following administration of the composition.

In one embodiment, the total daily dose of aspirin is about 160 mg that is released from the composition based on an aspirin release profile, wherein 80% of aspirin AUC is released over a period of about 4 hours following administration of the composition. In another embodiment, the total daily dose of aspirin is about 160 mg that is released from the composition based on an aspirin release profile, wherein 90% of aspirin AUC is released over a period of about 4 hours following administration of the composition. In another embodiment, the total daily dose of aspirin is released from the composition based on an aspirin release profile, wherein aspirin concentration in plasma is greater than 10% of Cmax over a period of time of about 4 hours following administration of the composition. In yet another embodiment, the total daily dose of aspirin is released from the composition based on an aspirin release profile, wherein aspirin concentration in plasma is greater than 20% of Cmax over a period of time of about 4 hours following administration of the composition.

In one embodiment, the total daily dose of aspirin is about 160 mg that is released from the composition based on an aspirin release profile, wherein 80% of aspirin AUC is released over a period of about 2-3 hours following administration of the composition. In another embodiment, the total daily dose of aspirin is about 160 mg that is released from the composition based on an aspirin release profile, wherein 90% of aspirin AUC is released over a period of about 2-3 hours following administration of the composition. In another embodiment, the total daily dose of aspirin is released from the composition based on an aspirin release profile, wherein aspirin concentration in plasma is greater than 10% of Cmax over a period of time of about 2-3 hours following administration of the composition. In yet another embodiment, the total daily dose of aspirin is released from the composition based on an aspirin release profile, wherein aspirin concentration in plasma is greater than 20% of Cmax over a period of time of about 2-3 hours following administration of the composition.

In one embodiment, a subject is predosed on the day of niacin therapy with a total daily dose of aspirin of about 160 mg, wherein about 80% of niacin AUC is not released from the composition until about 2 hour to about 11 hours of predosing with aspirin. In another embodiment, about 90% of niacin is not released from the composition until about 2 hour to about 11 hours of predosing with aspirin. In another embodiment, the plasma concentration of niacin is less than 20% of Cmax until about 2 hour to about 11 hours of predosing with the total daily dose of aspirin of about 160 mg. In yet another embodiment, the plasma concentration of niacin is less than 10% of Cmax until about 2 hour to about 11 hours of predosing with the total daily dose of aspirin of about 160 mg.

In one embodiment, the total daily dose of aspirin is about 162 mg that is released from the composition based on an aspirin release profile, wherein 80% of aspirin AUC is released over a period of about 8 hours following administration of the composition. In another embodiment, the total daily dose of aspirin is about 162 mg that is released from the composition based on an aspirin release profile, wherein 90% of aspirin AUC is released over a period of about 8 hours following administration of the composition. In another embodiment, the total daily dose of aspirin is released from the composition based on an aspirin release profile, wherein aspirin concentration in plasma is greater than 5% of Cmax over a period of time of about 8 hours following administration of the composition. In yet another embodiment, the total daily dose of aspirin is released from the composition based on an aspirin release profile, wherein aspirin concentration in plasma is greater than 10% of Cmax over a period of time of about 8 hours following administration of the composition.

In one embodiment, the total daily dose of aspirin is about 162 mg that is released from the composition based on an aspirin release profile, wherein 80% of aspirin AUC is released over a period of about 5-6 hours following administration of the composition. In another embodiment, the total daily dose of aspirin is about 162 mg that is released from the composition based on an aspirin release profile, wherein 90% of aspirin AUC is released over a period of about 5-6 hours following administration of the composition. In another embodiment, the total daily dose of aspirin is released from the composition based on an aspirin release profile, wherein aspirin concentration in plasma is greater than 5% of Cmax over a period of time of about 5-6 hours following administration of the composition. In yet another embodiment, the total daily dose of aspirin is released from the composition based on an aspirin release profile, wherein aspirin concentration in plasma is greater than 10% of Cmax over a period of time of about 5-6 hours following administration of the composition.

In one embodiment, the total daily dose of aspirin is about 162 mg that is released from the composition based on an aspirin release profile, wherein 80% of aspirin AUC is released over a period of about 4 hours following administration of the composition. In another embodiment, the total daily dose of aspirin is about 162 mg that is released from the composition based on an aspirin release profile, wherein 90% of aspirin AUC is released over a period of about 4 hours following administration of the composition. In another embodiment, the total daily dose of aspirin is released from the composition based on an aspirin release profile, wherein aspirin concentration in plasma is greater than 10% of Cmax over a period of time of about 4 hours following administration of the composition. In yet another embodiment, the total daily dose of aspirin is released from the composition based on an aspirin release profile, wherein aspirin concentration in plasma is greater than 20% of Cmax over a period of time of about 4 hours following administration of the composition.

In one embodiment, the total daily dose of aspirin is about 162 mg that is released from the composition based on an aspirin release profile, wherein 80% of aspirin AUC is released over a period of about 2-3 hours following administration of the composition. In another embodiment, the total daily dose of aspirin is about 162 mg that is released from the composition based on an aspirin release profile, wherein 90% of aspirin AUC is released over a period of about 2-3 hours following administration of the composition. In another embodiment, the total daily dose of aspirin is released from the composition based on an aspirin release profile, wherein aspirin concentration in plasma is greater than 10% of Cmax over a period of time of about 2-3 hours following administration of the composition. In yet another embodiment, the total daily dose of aspirin is released from the composition based on an aspirin release profile, wherein aspirin concentration in plasma is greater than 20% of Cmax over a period of time of about 2-3 hours following administration of the composition.

In one embodiment, a subject is predosed on the day of niacin therapy with a total daily dose of aspirin of about 162 mg, wherein about 80% of niacin AUC is not released from the composition until about 2 hour to about 11 hours of predosing with aspirin. In another embodiment, about 90% of niacin AUC is not released from the composition until about 2 hour to about 11 hours of predosing with aspirin. In another embodiment, the plasma concentration of niacin is less than 20% of Cmax until about 2 hour to about 11 hours of predosing with the total daily dose of aspirin of 162 mg. In yet another embodiment, the plasma concentration of niacin is less than 10% of Cmax until about 2 hour to about 11 hours of predosing with the total daily dose of aspirin of 162 mg.

In one embodiment, the total daily dose of aspirin is about 240 mg that is released from the composition based on an aspirin release profile, wherein 80% of aspirin AUC is released over a period of about 12 hours following administration of the composition. In another embodiment, the total daily dose of aspirin is about 240 mg that is released from the composition based on an aspirin release profile, wherein 90% of aspirin AUC is released over a period of about 12 hours following administration of the composition. In another embodiment, the total daily dose of aspirin is released from the composition based on an aspirin release profile, wherein aspirin concentration in plasma is greater than 5% of Cmax over a period of time of about 12 hours following administration of the composition. In yet another embodiment, the total daily dose of aspirin is released from the composition based on an aspirin release profile, wherein aspirin concentration in plasma is greater than 10% of Cmax over a period of time of about 12 hours following administration of the composition.

In one embodiment, the total daily dose of aspirin is about 240 mg that is released from the composition based on an aspirin release profile, wherein 80% of aspirin AUC is released over a period of about 8 hours following administration of the composition. In another embodiment, the total daily dose of is about 240 mg that is released from the composition based on an aspirin release profile, wherein 90% of aspirin AUC is released over a period of about 8 hours following administration of the composition. In another embodiment, the total daily dose of aspirin is released from the composition based on an aspirin release profile, wherein aspirin concentration in plasma is greater than 5% of Cmax over a period of time of about 8 hours following administration of the composition. In yet another embodiment, the total daily dose of aspirin is released from the composition based on an aspirin release profile, wherein aspirin concentration in plasma is greater than 10% of Cmax over a period of time of about 8 hours following administration of the composition.

In one embodiment, the total daily dose of aspirin is about 240 mg that is released from the composition based on an aspirin release profile, wherein 80% of aspirin AUC is released over a period of about 6 hours following administration of the composition. In another embodiment, the total daily dose of aspirin is about 240 mg that is released from the composition based on an aspirin release profile, wherein 90% of aspirin AUC is released over a period of about 6 hours following administration of the composition. In another embodiment, the total daily dose of aspirin is released from the composition based on an aspirin release profile, wherein aspirin concentration in plasma is greater than 5% of Cmax over a period of time of about 6 hours following administration of the composition. In yet another embodiment, the total daily dose of aspirin is released from the composition based on an aspirin release profile, wherein aspirin concentration in plasma is greater than 10% of Cmax over a period of time of about 6 hours following administration of the composition.

In one embodiment, the total daily dose of aspirin is about 240 mg that is released from the composition based on an aspirin release profile, wherein 80% of aspirin AUC is released over a period of about 4 hours following administration of the composition. In another embodiment, the total daily dose of aspirin is about 240 mg that is released from the composition based on an aspirin release profile, wherein 90% of aspirin AUC is released over a period of about 4 hours following administration of the composition. In another embodiment, the total daily dose of aspirin is released from the composition based on an aspirin release profile, wherein aspirin concentration in plasma is greater than 10% of Cmax over a period of time of about 4 hours following administration of the composition. In yet another embodiment, the total daily dose of aspirin is released from the composition based on an aspirin release profile, wherein aspirin concentration in plasma is greater than 20% of Cmax over a period of time of about 4 hours following administration of the composition.

In one embodiment, a subject is predosed on the day of niacin therapy with a total daily dose of aspirin of about 240 mg, wherein about 80% of niacin AUC is not released from the composition until about 2 hour to about 16 hours of predosing with aspirin. In another embodiment, about 90% of niacin AUC is not released from the composition until about 2 hour to about 16 hours of predosing with aspirin. In another embodiment, the plasma concentration of niacin is less than 20% of Cmax until about 2 hour to about 16 hours of predosing with the total daily dose of aspirin of 240 mg. In yet another embodiment, the plasma concentration of niacin is less than 10% of Cmax until about 2 hour to about 16 hours of predosing with the total daily dose of aspirin of 240 mg.

In one embodiment, the total daily dose of aspirin is about 243 mg that is released from the composition based on an aspirin release profile, wherein 80% of aspirin AUC is released over a period of about 12 hours following administration of the composition. In another embodiment, the total daily dose of aspirin is about 243 mg that is released from the composition based on an aspirin release profile, wherein 90% of aspirin AUC is released over a period of about 12 hours following administration of the composition. In another embodiment, the total daily dose of aspirin is released from the composition based on an aspirin release profile, wherein aspirin concentration in plasma is greater than 5% of Cmax over a period of time of about 12 hours following administration of the composition. In yet another embodiment, the total daily dose of aspirin is released from the composition based on an aspirin release profile, wherein aspirin concentration in plasma is greater than 10% of Cmax over a period of time of about 12 hours following administration of the composition.

In one embodiment, the total daily dose of aspirin is about 243 mg that is released from the composition based on an aspirin release profile, wherein 80% of aspirin AUC is released over a period of about 8 hours following administration of the composition. In another embodiment, the total daily dose of aspirin is about 243 mg that is released from the composition based on an aspirin release profile, wherein 90% of aspirin AUC is released over a period of about 8 hours following administration of the composition. In another embodiment, the total daily dose of aspirin is released from the composition based on an aspirin release profile, wherein aspirin concentration in plasma is greater than 5% of Cmax over a period of time of about 8 hours following administration of the composition. In yet another embodiment, the total daily dose of aspirin is released from the composition based on an aspirin release profile, wherein aspirin concentration in plasma is greater than 10% of Cmax over a period of time of about 8 hours following administration of the composition.

In one embodiment, the total daily dose of aspirin AUC is about 243 mg that is released from the composition based on an aspirin release profile, wherein 80% of aspirin AUC is released over a period of about 6 hours following administration of the composition. In another embodiment, the total daily dose of aspirin AUC is about 243 mg that is released from the composition based on an aspirin release profile, wherein 90% of aspirin AUC is released over a period of about 6 hours following administration of the composition. In another embodiment, the total daily dose of aspirin AUC is released from the composition based on an aspirin release profile, wherein aspirin concentration in plasma is greater than 5% of Cmax over a period of time of about 6 hours following administration of the composition. In yet another embodiment, the total daily dose of aspirin AUC is released from the composition based on an aspirin release profile, wherein aspirin concentration in plasma is greater than 10% of Cmax over a period of time of about 6 hours following administration of the composition.

In one embodiment, the total daily dose of aspirin is about 243 mg that is released from the composition based on an aspirin release profile, wherein 80% of aspirin AUC is released over a period of about 4 hours following administration of the composition. In another embodiment, the total daily dose of aspirin is about 243 mg that is released from the composition based on an aspirin release profile, wherein 90% of aspirin AUC is released over a period of about 4 hours following administration of the composition. In another embodiment, the total daily dose of aspirin is released from the composition based on an aspirin release profile, wherein aspirin concentration in plasma is greater than 10% of Cmax over a period of time of about 4 hours following administration of the composition. In yet another embodiment, the total daily dose of aspirin is released from the composition based on an aspirin release profile, wherein aspirin concentration in plasma is greater than 20% of Cmax over a period of time of about 4 hours following administration of the composition.

In one embodiment, a subject is predosed on the day of niacin therapy with a total daily dose of aspirin of about 243 mg, wherein about 80% of niacin AUC is not released from the composition until about 2 hour to about 16 hours of predosing with aspirin. In another embodiment, about 90% of niacin AUC is not released from the composition until about 2 hour to about 16 hours of predosing with aspirin. In another embodiment, the plasma concentration of niacin is less than 20% of Cmax until about 2 hour to about 16 hours of predosing with the total daily dose of aspirin of 243 mg. In yet another embodiment, the plasma concentration of niacin is less than 10% of Cmax until about 2 hour to about 16 hours of predosing with the total daily dose of aspirin of 243 mg.

In one embodiment, the total daily dose of aspirin is about 320 mg that is released from the composition based on an aspirin release profile, wherein 80% of aspirin AUC is released over a period of about 16 hours following administration of the composition. In another embodiment, the total daily dose of aspirin is about 320 mg that is released from the composition based on an aspirin release profile, wherein 90% of aspirin AUC is released over a period of about 16 hours following administration of the composition. In another embodiment, the total daily dose of aspirin is released from the composition based on an aspirin release profile, wherein aspirin concentration in plasma is greater than 5% of Cmax over a period of time of about 16 hours following administration of the composition. In yet another embodiment, the total daily dose of aspirin is released from the composition based on an aspirin release profile, wherein aspirin concentration in plasma is greater than 10% of Cmax over a period of time of about 16 hours following administration of the composition.

In one embodiment, the total daily dose of aspirin is about 320 mg that is released from the composition based on an aspirin release profile, wherein 80% of aspirin AUC is released over a period of about 10-11 hours following administration of the composition. In another embodiment, the total daily dose of aspirin is about 320 mg that is released from the composition based on an aspirin release profile, wherein 90% of aspirin AUC is released over a period of about 10-11 hours following administration of the composition. In another embodiment, the total daily dose of aspirin is released from the composition based on an aspirin release profile, wherein aspirin concentration in plasma is greater than 5% of Cmax over a period of time of about 10-11 hours following administration of the composition. In yet another embodiment, the total daily dose of aspirin is released from the composition based on an aspirin release profile, wherein aspirin concentration in plasma is greater than 10% of Cmax over a period of time of about 10-11 hours following administration of the composition.

In one embodiment, the total daily dose of aspirin is about 320 mg that is released from the composition based on an aspirin release profile, wherein 80% of aspirin AUC is released over a period of about 8 hours following administration of the composition. In another embodiment, the total daily dose of aspirin is about 320 mg that is released from the composition based on an aspirin release profile, wherein 90% of aspirin AUC is released over a period of about 8 hours following administration of the composition. In another embodiment, the total daily dose of aspirin is released from the composition based on an aspirin release profile, wherein aspirin concentration in plasma is greater than 5% of Cmax over a period of time of about 8 hours following administration of the composition. In yet another embodiment, the total daily dose of aspirin is released from the composition based on an aspirin release profile, wherein aspirin concentration in plasma is greater than 10% of Cmax over a period of time of about 8 hours following administration of the composition.

In one embodiment, the total daily dose of aspirin is about 320 mg that is released from the composition based on an aspirin release profile, wherein 80% of aspirin AUC is released over a period of about 5-6 hours following administration of the composition. In another embodiment, the total daily dose of aspirin is about 320 mg that is released from the composition based on an aspirin release profile, wherein 90% of aspirin AUC is released over a period of about 5-6 hours following administration of the composition. In another embodiment, the total daily dose of aspirin is released from the composition based on an aspirin release profile, wherein aspirin concentration in plasma is greater than 5% of Cmax over a period of time of about 5-6 hours following administration of the composition. In yet another embodiment, the total daily dose of aspirin is released from the composition based on an aspirin release profile, wherein aspirin concentration in plasma is greater than 10% of Cmax over a period of time of about 5-6 hours following administration of the composition.

In one embodiment, the total daily dose of aspirin is about 320 mg that is released from the composition based on an aspirin release profile, wherein 80% of aspirin AUC is released over a period of about 4 hours following administration of the composition. In another embodiment, the total daily dose of aspirin is about 320 mg that is released from the composition based on an aspirin release profile, wherein 90% of aspirin AUC is released over a period of about 4 hours following administration of the composition. In another embodiment, the total daily dose of aspirin is released from the composition based on an aspirin release profile, wherein aspirin concentration in plasma is greater than 10% of Cmax over a period of time of about 4 hours following administration of the composition. In yet another embodiment, the total daily dose of aspirin is released from the composition based on an aspirin release profile, wherein aspirin concentration in plasma is greater than 20% of Cmax over a period of time of about 4 hours following administration of the composition.

In one embodiment, a subject is predosed on the day of niacin therapy with a total daily dose of aspirin of about 320 mg, wherein about 80% of niacin AUC is not released from the composition until about 2 hour to about 16 hours of predosing with aspirin. In another embodiment, about 90% of niacin AUC is not released from the composition until about 2 hour to about 16 hours of predosing with aspirin. In another embodiment, the plasma concentration of niacin is less than 20% of Cmax until about 2 hour to about 16 hours of predosing with the total daily dose of aspirin of 320 mg. In yet another embodiment, the plasma concentration of niacin is less than 10% of Cmax until about 2 hour to about 16 hours of predosing with the total daily dose of aspirin of 320 mg.

In one embodiment, the total daily dose of aspirin is about 324 mg that is released from the composition based on an aspirin release profile, wherein 80% of aspirin AUC is released over a period of about 16 hours following administration of the composition. In another embodiment, the total daily dose of aspirin is about 324 mg that is released from the composition based on an aspirin release profile, wherein 90% of aspirin AUC is released over a period of about 16 hours following administration of the composition. In another embodiment, the total daily dose of aspirin is released from the composition based on an aspirin release profile, wherein aspirin concentration in plasma is greater than 5% of Cmax over a period of time of about 16 hours following administration of the composition. In yet another embodiment, the total daily dose of aspirin is released from the composition based on an aspirin release profile, wherein aspirin concentration in plasma is greater than 10% of Cmax over a period of time of about 16 hours following administration of the composition.

In one embodiment, the total daily dose of aspirin is about 324 mg that is released from the composition based on an aspirin release profile, wherein 80% of aspirin AUC is released over a period of about 10-11 hours following administration of the composition. In another embodiment, the total daily dose of aspirin is about 324 mg that is released from the composition based on an aspirin release profile, wherein 90% of aspirin AUC is released over a period of about 10-11 hours following administration of the composition. In another embodiment, the total daily dose of aspirin is released from the composition based on an aspirin release profile, wherein aspirin concentration in plasma is greater than 5% of Cmax over a period of time of about 10-11 hours following administration of the composition. In yet another embodiment, the total daily dose of aspirin is released from the composition based on an aspirin release profile, wherein aspirin concentration in plasma is greater than 10% of Cmax over a period of time of about 10-11 hours following administration of the composition.

In one embodiment, the total daily dose of aspirin A is about 324 mg that is released from the composition based on an aspirin release profile, wherein 80% of aspirin AUC is released over a period of about 8 hours following administration of the composition. In another embodiment, the total daily dose of aspirin A is about 324 mg that is released from the composition based on an aspirin release profile, wherein 90% of aspirin AUC is released over a period of about 8 hours following administration of the composition. In another embodiment, the total daily dose of aspirin is released from the composition based on an aspirin release profile, wherein aspirin concentration in plasma is greater than 5% of Cmax over a period of time of about 8 hours following administration of the composition. In yet another embodiment, the total daily dose of aspirin is released from the composition based on an aspirin release profile, wherein aspirin concentration in plasma is greater than 10% of Cmax over a period of time of about 8 hours following administration of the composition.

In one embodiment, the total daily dose of aspirin is about 324 mg that is released from the composition based on an aspirin release profile, wherein 80% of aspirin AUC is released over a period of about 5-6 hours following administration of the composition. In another embodiment, the total daily dose of aspirin is about 324 mg that is released from the composition based on an aspirin release profile, wherein 90% of aspirin AUC is released over a period of about 5-6 hours following administration of the composition. In another embodiment, the total daily dose of aspirin is released from the composition based on an aspirin release profile, wherein aspirin concentration in plasma is greater than 5% of Cmax over a period of time of about 5-6 hours following administration of the composition. In yet another embodiment, the total daily dose of aspirin is released from the composition based on an aspirin release profile, wherein aspirin concentration in plasma is greater than 10% of Cmax over a period of time of about 5-6 hours following administration of the composition.

In one embodiment, the total daily dose of aspirin is about 324 mg that is released from the composition based on an aspirin release profile, wherein 80% of aspirin AUC is released over a period of about 4 hours following administration of the composition. In another embodiment, the total daily dose of aspirin is about 324 mg that is released from the composition based on an aspirin release profile, wherein 90% of aspirin AUC is released over a period of about 4 hours following administration of the composition. In another embodiment, the total daily dose of aspirin is released from the composition based on an aspirin release profile, wherein aspirin concentration in plasma is greater than 10% of Cmax over a period of time of about 4 hours following administration of the composition. In yet another embodiment, the total daily dose of aspirin is released from the composition based on an aspirin release profile, wherein aspirin concentration in plasma is greater than 20% of Cmax over a period of time of about 4 hours following administration of the composition.

In one embodiment, a subject is predosed on the day of niacin therapy with a total daily dose of aspirin of about 324 mg, wherein about 80% of niacin AUC is not released from the composition until about 2 hour to about 16 hours of predosing with aspirin. In another embodiment, about 90% of niacin AUC is not released from the composition until about 2 hour to about 16 hours of predosing with aspirin. In another embodiment, the plasma concentration of niacin is less than 20% of Cmax until about 2 hour to about 16 hours of predosing with the total daily dose of aspirin of 324 mg. In yet another embodiment, the plasma concentration of niacin is less than 10% of Cmax until about 2 hour to about 16 hours of predosing with the total daily dose of aspirin of 324 mg.

In one embodiment, the total daily dose of aspirin is about 81 mg and it is slow and extended released from the combination formulation or formulation, while the niacin amount is 250 mg and is delayed released according to other embodiments provided herein.

In one embodiment, the total daily dose of aspirin is about 81 mg and it is an extended release formulation, while the niacin amount is 500 mg and is delayed released according to other embodiments provided herein.

In one embodiment, the total daily dose of aspirin is about 81 mg and it is slow and extended released from the combination formulation or formulation, while the niacin amount is 1000 mg and is delayed released according to other embodiments provided herein.

In one embodiment, the total daily dose of aspirin is about 81 mg and it is slow and extended released from the combination formulation or formulation, while the niacin amount is 1500 mg and is delayed released according to other embodiments provided herein.

In one embodiment the total daily dose of aspirin is about 81 mg and it is slow and extended released from the combination formulation or formulation, while the niacin amount is 2000 mg and is delayed released according to other embodiments provided herein.

In one embodiment, the total daily dose of aspirin is about 162 mg and it is slow and extended released from the combination formulation or formulation, while the niacin amount is 250 mg and is delayed released according to other embodiments provided herein.

In one embodiment, the total daily dose of aspirin is about 162 mg and it is slow and extended released from the combination formulation or formulation, while the niacin amount is 500 mg and is delayed released according to other embodiments provided herein.

In one embodiment, the total daily dose of aspirin is about 162 mg and it is slow and extended released from the combination formulation or formulation, while the niacin amount is 1000 mg and is delayed released according to other embodiments provided herein.

In one embodiment, the total daily dose of aspirin is about 162 mg and it is slow and extended released from the combination formulation or formulation, while the niacin amout is 1500 mg and is delayed released according to other embodiments provided herein.

In one embodiment the total daily dose of aspirin is about 162 mg and it is slow and extended released from the combination formulation or formulation, while the niacin amout is 2000 mg and is delayed released according to other embodiments provided herein.

In one embodiment, the total daily dose of aspirin is about 243 mg and it is slow and extended released from the combination formulation or formulation, while the niacin amout is 250 mg and is delayed released according to other embodiments provided herein.

In one embodiment, the total daily dose of aspirin is about 243 mg and it is slow and extended released from the combination formulation or formulation, while the niacin amout is 500 mg and is delayed released according to other embodiments provided herein.

In one embodiment, the total daily dose of aspirin is about 243 mg and it is slow and extended released from the combination formulation or formulation, while the niacin amout is 1000 mg and is delayed released according to other embodiments provided herein.

In one embodiment, the total daily dose of aspirin is about 243 mg and it is slow and extended released from the combination formulation or formulation, while the niacin amout is 1500 mg and is delayed released according to other embodiments provided herein.

In one embodiment the total daily dose of aspirin is about 243 mg and it is slow and extended released from the combination formulation or formulation, while the niacin amout is 2000 mg and is delayed released according to other embodiments provided herein.

In one embodiment, the total daily dose of aspirin is about 324 mg and it is slow and extended released from the combination formulation or formulation, while the niacin amout is 250 mg and is delayed released according to other embodiments provided herein.

In one embodiment, the total daily dose of aspirin is about 324 mg and it is slow and extended released from the combination formulation or formulation, while the niacin amout is 500 mg and is delayed released according to other embodiments provided herein.

In one embodiment, the total daily dose of aspirin is about 324 mg and it is slow and extended released from the combination formulation or formulation, while the niacin amout is 1000 mg and is delayed released according to other embodiments provided herein.

In one embodiment, the total daily dose of aspirin is about 324 mg and it is slow and extended released from the combination formulation or formulation, while the niacin amout is 1500 mg and is delayed released according to other embodiments provided herein.

In one embodiment the total daily dose of aspirin is about 324 mg and it is slow and extended released from the combination formulation or formulation, while the niacin amout is 2000 mg and is delayed released according to other embodiments provided herein.

In another embodiment, the total daily dose includes 1-7 days of aspirin pretreatment prior to the first niacin dose. In yet another embodiment, aspirin dosing includes 2-4 days of aspirin pretreatment. Such pretreatment may use an immediate or extended release aspirin formulation.

Provided herein are pharmaceutical compositions in modified release dosage forms, which comprise niacin and aspirin or a pharmaceutically acceptable salt, solvate, or prodrug thereof; and one or more release controlling excipients as described herein. Suitable modified release dosage vehicles include, but are not limited to, hydrophilic or hydrophobic matrix devices, water-soluble separating layer coatings, enteric coatings, osmotic devices, multiparticulate devices, and combinations thereof. The pharmaceutical compositions may also comprise non-release controlling excipients.

Further provided herein are pharmaceutical compositions in enteric coated dosage forms, which comprise niacin and aspirin or a pharmaceutically acceptable salt, solvate, or prodrug thereof; and one or more release controlling excipients for use in an enteric coated dosage form. The pharmaceutical compositions may also comprise non-release controlling excipients.

In one embodiment, the pharmaceutical compositions comprise niacin and aspirin or a pharmaceutically acceptable salt (See, Berge et al., J. Pharm. Sci. 1977, 66, 1-19; and “Handbook of Pharmaceutical Salts, Properties, and Use,” Stahl and Wermuth, Ed.; Wiley-VCH and VHCA, Zurich, 2002)., solvate, or prodrug thereof.

Suitable acids for use in the preparation of pharmaceutically acceptable salts include, but are not limited to, chloride, hydrochloride, acetic acid, 2,2-dichloroacetic acid, acylated amino acids, adipic acid, alginic acid, ascorbic acid, L-aspartic acid, benzenesulfonic acid, benzoic acid, 4-acetamidobenzoic acid, boric acid, (+)-camphoric acid, camphorsulfonic acid, (+)-(1S)-camphor-10-sulfonic acid, capric acid, caproic acid, caprylic acid, cinnamic acid, citric acid, cyclamic acid, cyclohexanesulfamic acid, dodecylsulfuric acid, ethane-1,2-disulfonic acid, ethanesulfonic acid, 2-hydroxy-ethanesulfonic acid, formic acid, fumaric acid, galactaric acid, gentisic acid, glucoheptonic acid, D-gluconic acid, D-glucuronic acid, L-glutamic acid, α-oxoglutaric acid, glycolic acid, hippuric acid, hydrobromic acid, hydrochloric acid, hydroiodic acid, (+)-L-lactic acid, (±)-DL-lactic acid, lactobionic acid, lauric acid, maleic acid, (−)-L-malic acid, malonic acid, (±)-DL-mandelic acid, methanesulfonic acid, naphthalene-2-sulfonic acid, naphthalene-1,5-disulfonic acid, 1-hydroxy-2-naphthoic acid, nicotinic acid, nitric acid, oleic acid, orotic acid, oxalic acid, palmitic acid, pamoic acid, perchloric acid, phosphoric acid, L-pyroglutamic acid, saccharic acid, salicylic acid, 4-amino-salicylic acid, sebacic acid, stearic acid, succinic acid, sulfuric acid, tannic acid, (+)-L-tartaric acid, thiocyanic acid, p-toluenesulfonic acid, undecylenic acid, and valeric acid.

Suitable bases for use in the preparation of pharmaceutically acceptable salts, including, but not limited to, inorganic bases, such as magnesium hydroxide, calcium hydroxide, potassium hydroxide, zinc hydroxide, or sodium hydroxide; and organic bases, such as primary, secondary, tertiary, and quaternary, aliphatic and aromatic amines, including L-arginine, benethamine, benzathine, choline, deanol, diethanolamine, diethylamine, dimethylamine, dipropylamine, diisopropylamine, 2-(diethylamino)-ethanol, ethanolamine, ethylamine, ethylenediamine, isopropylamine, N-methyl-glucamine, hydrabamine, 1H-imidazole, L-lysine, morpholine, 4-(2-hydroxyethyl)-morpholine, methylamine, piperidine, piperazine, propylamine, pyrrolidine, 1-(2-hydroxyethyl)-pyrrolidine, pyridine, quinuclidine, quinoline, isoquinoline, secondary amines, triethanolamine, trimethylamine, triethylamine, N-methyl-D-glucamine, 2-amino-2-(hydroxymethyl)-1,3-propanediol, and tromethamine.

Prodrugs are functional derivative of the compounds, and are readily convertible into the parent compound in vivo. Prodrugs are often useful because, in some situations, they may be easier to administer than the parent compound. They may, for instance, be bioavailable by oral administration whereas the parent compound is not. The prodrug may also have enhanced solubility in pharmaceutical compositions over the parent compound or pharmacokinetic properties. A prodrug may be converted into the parent drug by various mechanisms, including enzymatic processes and metabolic hydrolysis. See Harper, Progress in Drug Research 1962, 4, 221-294; Morozowich et al. in “Design of Biopharmaceutical Properties through Prodrugs and Analogs,” Roche Ed., APHA Acad. Pharm. Sci. 1977; “Bioreversible Carriers in Drug in Drug Design, Theory and Application,” Roche Ed., APHA Acad. Pharm. Sci. 1987; “Design of Prodrugs,” Bundgaard, Elsevier, 1985; Wang et al., Curr. Pharm. Design 1999, 5, 265-287; Pauletti et al., Adv. Drug. Delivery Rev. 1997, 27, 235-256; Mizen et al., Pharm. Biotech. 1998, 11, 345-365; Gaignault et al., Pract. Med. Chem. 1996, 671-696; Asgharnejad in “Transport Processes in Pharmaceutical Systems,” Amidon et al., Ed., Marcell Dekker, 185-218, 2000; Balant et al., Eur. J. Drug Metab. Pharmacokinet. 1990, 15, 143-53; Balimane and Sinko, Adv. Drug Delivery Rev. 1999, 39, 183-209; Browne, Clin. Neuropharmacol. 1997, 20, 1-12; Bundgaard, Arch. Pharm. Chem. 1979, 86, 1-39; Bundgaard, Controlled Drug Delivery 1987, 17, 179-96; Bundgaard, Adv. Drug Delivery Rev. 1992, 8, 1-38; Fleisher et al., Adv. Drug Delivery Rev. 1996, 19, 115-130; Fleisher et al., Methods Enzymol. 1985, 112, 360-381; Farquhar et al., J. Pharm. Sci. 1983, 72, 324-325; Freeman et al., J. Chem. Soc., Chem. Commun. 1991, 875-877; Friis and Bundgaard, Eur. J. Pharm. Sci. 1996, 4, 49-59; Gangwar et al., Des. Biopharm. Prop. Prodrugs Analogs, 1977, 409-421; Nathwani and Wood, Drugs 1993, 45, 866-94; Sinhababu and Thakker, Adv. Drug Delivery Rev. 1996, 19, 241-273; Stella et al., Drugs 1985, 29, 455-73; Tan et al., Adv. Drug Delivery Rev. 1999, 39, 117-151; Taylor, Adv. Drug Delivery Rev. 1996, 19, 131-148; Valentino and Borchardt, Drug Discovery Today 1997, 2, 148-155; Wiebe and Knaus, Adv. Drug Delivery Rev. 1999, 39, 63-80; and Waller et al., Br. J. Clin. Pharmac. 1989, 28, 497-507.

In another embodiment, the pharmaceutical compositions comprise niacin and aspirin and one or more release controlling and non-release controlling excipients, such as those excipients suitable for a disruptable semi-permeable membrane and as swellable or effervescent substances.

Provided herein are also pharmaceutical compositions in a dosage form for oral administration to a subject, which comprise niacin and or a pharmaceutically acceptable salt, solvate, or prodrug thereof; and one or more pharmaceutically acceptable excipients or carriers, enclosed in an intermediate reactive layer comprising a gastric juice-resistant polymeric layered material partially neutralized with alkali and having cation exchange capacity and a gastric juice-resistant outer layer.

The pharmaceutical compositions provided herein may be provided in unit-dosage forms or multiple-dosage forms. Unit-dosage forms, as used herein, refer to physically discrete units suitable for administration to human and animal subjects and packaged individually as is known in the art. Each unit-dose contains a predetermined quantity of the active ingredient(s) sufficient to produce the desired therapeutic effect, in association with the required pharmaceutical carriers or excipients. Examples of unit-dosage forms include ampoules, syringes, and individually packaged tablets and capsules. Unit-dosage forms may be administered in fractions or multiples thereof. A multiple-dosage form is a plurality of identical unit-dosage forms packaged in a single container to be administered in segregated unit-dosage form. Examples of multiple-dosage forms include vials, bottles of tablets or capsules.

In certain embodiments, the individual dosage forms (tablets or capsules) comprise, for example, about 250 mg of niacin and about 80 mg of aspirin. In certain embodiments, the individual dosage forms comprise, for example, about 250 mg of niacin and about 120 mg of aspirin. In certain embodiments, the individual dosage forms comprise, for example, about 250 mg of niacin and about 160 mg of aspirin. In certain embodiments, the individual dosage forms comprise, for example, about 250 mg of niacin and about 240 mg of aspirin. In certain embodiments, the individual dosage forms comprise, for example, about 250 mg of niacin and about 320 mg of aspirin.

In certain embodiments, the individual dosage forms (tablets or capsules) comprise, for example, about 333 mg of niacin and about 53 mg of aspirin. In certain embodiments, the individual dosage forms comprise, for example, about 333 mg of niacin and about 80 mg of aspirin. In certain embodiments, the individual dosage forms comprise, for example, about 333 mg of niacin and about 107 mg of aspirin. In certain embodiments, the individual dosage forms comprise, for example, about 333 mg of niacin and about 160 mg of aspirin. In certain embodiments, the individual dosage forms comprise, for example, about 333 mg of niacin and about 240 mg of aspirin. In certain embodiments, the individual dosage forms comprise, for example, about 333 mg of niacin and about 320 mg of aspirin.

In certain embodiments, the individual dosage forms (tablets or capsules) comprise, for example, about 500 mg of niacin and about 40 mg of aspirin. In certain embodiments, the individual dosage forms comprise, for example, about 500 mg of niacin and about 60 mg of aspirin. In certain embodiments, the individual dosage forms comprise, for example, about 500 mg of niacin and about 80 mg of aspirin. In certain embodiments, the individual dosage forms comprise, for example, about 500 mg of niacin and about 81 mg of aspirin. In certain embodiments, the individual dosage forms comprise, for example, about 500 mg of niacin and about 120 mg of aspirin. In certain embodiments, the individual dosage forms comprise, for example, about 500 mg of niacin and about 160 mg of aspirin. In certain embodiments, the individual dosage forms comprise, for example, about 500 mg of niacin and about 162 mg of aspirin. In certain embodiments, the individual dosage forms comprise, for example, about 500 mg of niacin and about 240 mg of aspirin. In certain embodiments, the individual dosage forms comprise, for example, about 500 mg of niacin and about 243 mg of aspirin. In certain embodiments, the individual dosage forms comprise, for example, about 500 mg of niacin and about 320 mg of aspirin. In certain embodiments, the individual dosage forms comprise, for example, about 500 mg of niacin and about 324 mg of aspirin. In certain embodiments, the individual dosage forms comprise, for example, about 500 mg of niacin and about 400 mg of aspirin. In certain embodiments, the individual dosage forms comprise, for example, about 500 mg of niacin and about 500 mg of aspirin. In certain embodiments, the individual dosage forms comprise, for example, about 500 mg of niacin and about 650 mg of aspirin.

In certain embodiments, the individual dosage forms comprise, for example, about 750 mg of niacin and about 80 mg of aspirin. In certain embodiments, the individual dosage forms comprise, for example, about 750 mg of niacin and about 81 mg of aspirin. In certain embodiments, the individual dosage forms comprise, for example, about 750 mg of niacin and about 120 mg of aspirin. In certain embodiments, the individual dosage forms comprise, for example, about 750 mg of niacin and about 160 mg of aspirin In certain embodiments, the individual dosage forms comprise, for example, about 750 mg of niacin and about 162 mg of aspirin. In certain embodiments, the individual dosage forms comprise, for example, about 750 mg of niacin and about 240 mg of aspirin. In certain embodiments, the individual dosage forms comprise, for example, about 750 mg of niacin and about 243 mg of aspirin In certain embodiments, the individual dosage forms comprise, for example, about 750 mg of niacin and about 320 mg of aspirin. In certain embodiments, the individual dosage forms comprise, for example, about 750 mg of niacin and about 324 mg of aspirin.

In certain embodiments, the individual dosage forms comprise, for example, about 1000 mg of niacin and about 80 mg of aspirin. In certain embodiments, the individual dosage forms comprise, for example, about 1000 mg of niacin and about 120 mg of aspirin. In certain embodiments, the individual dosage forms comprise, for example, about 1000 mg of niacin and about 160 mg of aspirin. In certain embodiments, the individual dosage forms comprise, for example, about 1000 mg of niacin and about 240 mg of aspirin. In certain embodiments, the individual dosage forms comprise, for example, about 1000 mg of niacin and about 320 mg of aspirin.

In certain embodiments, the individual dosage forms comprise, for example, about 2000 mg of niacin and about 80 mg of aspirin. In certain embodiments, the individual dosage forms comprise, for example, about 2000 mg of niacin and about 120 mg of aspirin. In certain embodiments, the individual dosage forms comprise, for example, about 2000 mg of niacin and about 160 mg of aspirin. In certain embodiments, the individual dosage forms comprise, for example, about 1000 mg of niacin and about 240 mg of aspirin. In certain embodiments, the individual dosage forms comprise, for example, about 1000 mg of niacin and about 320 mg of aspirin.

Niacin and aspirin provided herein may be administered alone, or in combination with one or more other compounds provided herein, or one or more other active ingredients. The pharmaceutical compositions that comprise compounds provided herein may be formulated in various dosage forms for oral administration. The pharmaceutical compositions may also be formulated as a modified release dosage form, including delayed-, extended-, prolonged-, extended-, pulsatile-, controlled-, accelerated- and fast-, targeted-, programmed-release, and gastric retention dosage forms. These dosage forms can be prepared according to conventional methods and techniques known to those skilled in the art (see, Remington: The Science and Practice of Pharmacy, supra; Modified-Release Drug Deliver Technology, Rathbone et al., Eds., Drugs and the Pharmaceutical Science, Marcel Dekker, Inc.: New York, N.Y., 2002; Vol. 126).

The pharmaceutical compositions provided herein may be administered at once, or multiple times at intervals of time. It is understood that the precise dosage and duration of treatment may vary with the age, weight, and condition of the subject being treated, and may be determined empirically using known testing protocols or by extrapolation from in vivo or in vitro test or diagnostic data. It is further understood that for any particular individual, specific dosage regimens should be adjusted over time according to the individual need and the professional judgment of the person administering or supervising the administration of the formulations.

In one aspect, provided are compositions for administering a flush-inhibiting dose of aspirin and optionally a lipid-lowering drug other than niacin during the pretreatment period, as well as compositions comprising a flush-inhibiting dose of aspirin and optionally a lipid-lowering drug other than niacin, and a flush-provoking dose of niacin. The compositions comprising a flush-inhibiting dose of aspirin and optionally a lipid-lowering drug other than niacin provide a dose less than 85%, less than 80%, less than 75% or less than 70% of the usual anti-inflammatory dose of aspirin. In one embodiment, an individual dose delivers about 320 mg, 240 mg, 160 mg or 120 mg of aspirin. The above-single administration (individual) doses may be provided by one or multiple solid dosage units, e.g., one, or two, or three, or four, or five capsules or tablets may be needed to make up the total individual dose.

Modified Release

The pharmaceutical compositions provided herein may be formulated as a modified release dosage form. As used herein, the term “modified release” refers to a dosage form in which the rate or place of release of the active ingredient(s) is different from that of an immediate dosage form when administered by the same route. Modified release dosage forms include delayed-, extended-, prolonged-, extended-, pulsatile- or pulsed-, controlled-, accelerated- and fast-, targeted-, programmed-release, and gastric retention dosage forms. The pharmaceutical compositions in modified release dosage forms can be prepared using a variety of modified release devices and methods known to those skilled in the art, including, but not limited to, matrix controlled release devices, osmotic controlled release devices, multiparticulate controlled release devices, ion-exchange resins, enteric coatings, multilayered coatings, microspheres, liposomes, and combinations thereof. The release rate of the active ingredient(s) can also be modified by varying the particle sizes and polymorphorism of the active ingredient(s).

Examples of modified release include, but are not limited to, those described in U.S. Pat. Nos. 3,845,770; 3,916,899; 3,536,809; 3,598,123; 4,008,719; 5,674,533; 5,059,595; 5,591,767; 5,120,548; 5,073,543; 5,639,476; 5,354,556; 5,639,480; 5,733,566; 5,739,108; 5,891,474; 5,922,356; 5,972,891; 5,980,945; 5,993,855; 6,045,830; 6,087,324; 6,113,943; 6,197,350; 6,248,363; 6,264,970; 6,267,981; 6,376,461; 6,419,961; 6,589,548; 6,613,358; and 6,699,500.

Matrix-Controlled Release Devices

The pharmaceutical compositions provided herein in a modified release dosage form may be fabricated using a matrix-controlled release device known to those skilled in the art (see, Takada et al in “Encyclopedia of Controlled Drug Delivery,” Vol. 2, Mathiowitz ed., Wiley, 1999).

In one embodiment, the pharmaceutical composition provided herein in a modified release dosage form is formulated using an erodible matrix device, which is water-swellable, erodible, or soluble polymers, including synthetic polymers, and naturally occurring polymers and derivatives, such as polysaccharides and proteins.

Materials useful in forming an erodible matrix include, but are not limited to, chitin, chitosan, dextran, and pullulan; gum agar, gum arabic, gum karaya, locust bean gum, gum tragacanth, carrageenans, gum ghatti, guar gum, xanthan gum, and scleroglucan; starches, such as dextrin and maltodextrin; hydrophilic colloids, such as pectin; phosphatides, such as lecithin; alginates; propylene glycol alginate; gelatin; collagen; and cellulosics, such as ethyl cellulose (EC), methylethyl cellulose (MEC), carboxymethyl cellulose (CMC), CMEC, hydroxyethyl cellulose (HEC), hydroxypropyl cellulose (HPC), cellulose acetate (CA), cellulose propionate (CP), cellulose butyrate (CB), cellulose acetate butyrate (CAB), CAP, CAT, hydroxypropyl methyl cellulose (HPMC), HPMCP, HPMCAS, hydroxypropyl methyl cellulose acetate trimellitate (HPMCAT), and ethylhydroxy ethylcellulose (EHEC); polyvinyl pyrrolidone; polyvinyl alcohol; polyvinyl acetate; glycerol fatty acid esters; polyacrylamide; polyacrylic acid; copolymers of ethacrylic acid or methacrylic acid (EUDRAGIT®, Rohm America, Inc., Piscataway, N.J.); poly(2-hydroxyethyl-methacrylate); polylactides; copolymers of L-glutamic acid and ethyl-L-glutamate; degradable lactic acid-glycolic acid copolymers; poly-D-(−)-3-hydroxybutyric acid; and other acrylic acid derivatives, such as homopolymers and copolymers of butylmethacrylate, methylmethacrylate, ethylmethacrylate, ethylacrylate, (2-dimethylaminoethyl)methacrylate, and (trimethylaminoethyl)methacrylate chloride.

In another embodiment, the pharmaceutical compositions are formulated with a non-erodible matrix device. The active ingredient(s) is dissolved or dispersed in an inert matrix and is released primarily by diffusion through the inert matrix once administered. Materials suitable for use as a non-erodible matrix device included, but are not limited to, insoluble plastics, such as polyethylene, polypropylene, polyisoprene, polyisobutylene, polybutadiene, polymethylmethacrylate, polybutylmethacrylate, chlorinated polyethylene, polyvinylchloride, methyl acrylate-methyl methacrylate copolymers, ethylene-vinylacetate copolymers, ethylene/propylene copolymers, ethylene/ethyl acrylate copolymers, vinylchloride copolymers with vinyl acetate, vinylidene chloride, ethylene and propylene, ionomer polyethylene terephthalate, butyl rubber epichlorohydrin rubbers, ethylene/vinyl alcohol copolymer, ethylene/vinyl acetate/vinyl alcohol terpolymer, and ethylene/vinyloxyethanol copolymer, polyvinyl chloride, plasticized nylon, plasticized polyethyleneterephthalate, natural rubber, silicone rubbers, polydimethylsiloxanes, silicone carbonate copolymers, and hydrophilic polymers, such as ethyl cellulose, cellulose acetate, crospovidone, and cross-linked partially hydrolyzed polyvinyl acetate; and fatty compounds, such as carnauba wax, microcrystalline wax, and triglycerides.

In a matrix-controlled release system, the desired release kinetics can be controlled, for example, via the polymer type employed, the polymer viscosity, the particle sizes of the polymer and/or the active ingredient(s), the ratio of the active ingredient(s) versus the polymer, and other excipients in the compositions.

The pharmaceutical compositions provided herein in a modified release dosage form may be prepared by methods known to those skilled in the art, including direct compression, dry or wet granulation followed by compression, melt-granulation followed by compression.

All excipients used in the combination formulations provided herein are listed and their characteristics are described by pharmacopeias.

Osmotic-Controlled Release Devices

The pharmaceutical compositions provided herein in a modified release dosage form may be fabricated using an osmotic controlled release device, including one-chamber system, two-chamber system, asymmetric membrane technology (AMT), and extruding core system (ECS). In general, such devices have at least two components: (a) the core which contains the active ingredient(s); and (b) a semipermeable membrane with at least one delivery port, which encapsulates the core. The semipermeable membrane controls the influx of water to the core from an aqueous environment of use so as to cause drug release by extrusion through the delivery port(s).

In addition to the active ingredient(s), the core of the osmotic device optionally includes an osmotic agent, which creates a driving force for transport of water from the environment of use into the core of the device. One class of osmotic agents water-swellable hydrophilic polymers, which are also referred to as “osmopolymers” and “hydrogels,” including, but not limited to, hydrophilic vinyl and acrylic polymers, polysaccharides such as calcium alginate, polyethylene oxide (PEO), polyethylene glycol (PEG), polypropylene glycol (PPG), poly(2-hydroxyethyl methacrylate), poly(acrylic) acid, poly(methacrylic) acid, polyvinylpyrrolidone (PVP), crosslinked PVP, polyvinyl alcohol (PVA), PVA/PVP copolymers, PVA/PVP copolymers with hydrophobic monomers such as methyl methacrylate and vinyl acetate, hydrophilic polyurethanes containing large PEO blocks, sodium croscarmellose, carrageenan, hydroxyethyl cellulose (HEC), hydroxypropyl cellulose (HPC), hydroxypropyl methyl cellulose (HPMC), carboxymethyl cellulose (CMC) and carboxyethyl, cellulose (CEC), sodium alginate, polycarbophil, gelatin, xanthan gum, and sodium starch glycolate.

The other class of osmotic agents is comprised of osmogens, which are capable of imbibing water to affect an osmotic pressure gradient across the barrier of the surrounding coating. Suitable osmogens include, but are not limited to, inorganic salts, such as magnesium sulfate, magnesium chloride, calcium chloride, sodium chloride, lithium chloride, potassium sulfate, potassium phosphates, sodium carbonate, sodium sulfite, lithium sulfate, potassium chloride, and sodium sulfate; sugars, such as dextrose, fructose, glucose, inositol, lactose, maltose, mannitol, raffinose, sorbitol, sucrose, trehalose, and xylitol; organic acids, such as ascorbic acid, benzoic acid, fumaric acid, citric acid, maleic acid, sebacic acid, sorbic acid, adipic acid, edetic acid, glutamic acid, p-tolunesulfonic acid, succinic acid, and tartaric acid; urea; and mixtures thereof.

Osmotic agents of different dissolution rates may be employed to influence how rapidly the active ingredient(s) is initially delivered from the dosage form. For example, amorphous sugars, such as Mannogeme EZ (SPI Pharma, Lewes, Del.) can be used to provide faster delivery during the first couple of hours to promptly produce the desired therapeutic effect, and gradually and continually release of the remaining amount to maintain the desired level of therapeutic or prophylactic effect over an extended period of time. In this case, the active ingredient(s) is released at such a rate to replace the amount of the active ingredient metabolized and excreted.

The core may also include a wide variety of other excipients and carriers as described herein to enhance the performance of the dosage form or to promote stability or processing.

Materials useful in forming the semipermeable membrane include various grades of acrylics, vinyls, ethers, polyamides, polyesters, and cellulosic derivatives that are water-permeable and water-insoluble at physiologically relevant pHs, or are susceptible to being rendered water-insoluble by chemical alteration, such as crosslinking. Examples of suitable polymers useful in forming the coating, include plasticized, unplasticized, and reinforced cellulose acetate (CA), cellulose diacetate, cellulose triacetate, CA propionate, cellulose nitrate, cellulose acetate butyrate (CAB), CA ethyl carbamate, CAP, CA methyl carbamate, CA succinate, cellulose acetate trimellitate (CAT), CA dimethylaminoacetate, CA ethyl carbonate, CA chloroacetate, CA ethyl oxalate, CA methyl sulfonate, CA butyl sulfonate, CA p-toluene sulfonate, agar acetate, amylose triacetate, beta glucan acetate, beta glucan triacetate, acetaldehyde dimethyl acetate, triacetate of locust bean gum, hydroxlated ethylene-vinylacetate, EC, PEG, PPG, PEG/PPG copolymers, PVP, HEC, HPC, CMC, CMEC, HPMC, HPMCP, HPMCAS, HPMCAT, poly(acrylic) acids and esters and poly-(methacrylic) acids and esters and copolymers thereof, starch, dextran, dextrin, chitosan, collagen, gelatin, polyalkenes, polyethers, polysulfones, polyethersulfones, polystyrenes, polyvinyl halides, polyvinyl esters and ethers, natural waxes, and synthetic waxes.

Semipermeable membrane may also be a hydrophobic microporous membrane, wherein the pores are substantially filled with a gas and are not wetted by the aqueous medium but are permeable to water vapor, as disclosed in U.S. Pat. No. 5,798,119. Such hydrophobic but water-vapor permeable membrane are typically composed of hydrophobic polymers such as polyalkenes, polyethylene, polypropylene, polytetrafluoroethylene, polyacrylic acid derivatives, polyethers, polysulfones, polyethersulfones, polystyrenes, polyvinyl halides, polyvinylidene fluoride, polyvinyl esters and ethers, natural waxes, and synthetic waxes.

The delivery port(s) on the semipermeable membrane may be formed post-coating by mechanical or laser drilling. Delivery port(s) may also be formed in situ by erosion of a plug of water-soluble material or by rupture of a thinner portion of the membrane over an indentation in the core. In addition, delivery ports may be formed during coating process, as in the case of asymmetric membrane coatings of the type disclosed in U.S. Pat. Nos. 5,612,059 and 5,698,220.

The total amount of the active ingredient(s) released and the release rate can substantially be modulated via the thickness and porosity of the semipermeable membrane, the composition of the core, and the number, size, and position of the delivery ports.

The pharmaceutical compositions in an osmotic controlled-release dosage form may further comprise additional conventional excipients as described herein to promote performance or processing of the formulation.

The osmotic controlled-release dosage forms can be prepared according to conventional methods and techniques known to those skilled in the art (see, Remington: The Science and Practice of Pharmacy, supra; Santus and Baker, J. Controlled Release 1995, 35, 1-21; Verma et al., Drug Development and Industrial Pharmacy 2000, 26, 695-708; Verma et al., J. Controlled Release 2002, 79, 7-27).

In certain embodiments, the pharmaceutical compositions provided herein are formulated as AMT controlled-release dosage form, which comprises an asymmetric osmotic membrane that coats a core comprising the active ingredient(s) and other pharmaceutically acceptable excipients. See, U.S. Pat. No. 5,612,059 and WO 2002/17918. The AMT controlled-release dosage forms can be prepared according to conventional methods and techniques known to those skilled in the art, including direct compression, dry granulation, wet granulation, and a dip-coating method.

In certain embodiment, the pharmaceutical compositions provided herein are formulated as ESC controlled-release dosage form, which comprises an osmotic membrane that coats a core comprising the active ingredient(s), hydroxylethyl cellulose, and other pharmaceutically acceptable excipients.

All excipients used in the combination formulations provided herein are listed and their characteristics are described by pharmacopeias.

Multiparticulate-Controlled Release Devices

The pharmaceutical compositions provided herein in a modified release dosage form may be a fabricated multiparticulate-controlled release device, which comprises a multiplicity of particles, granules, or pellets, microparticulatesbeads, microcapsules and microtablets, ranging from about 10 μm to about 3 mm, about 50 μm to about 2.5 mm, or from about 100 μm to 1 mm in diameter.

The microspheres can provide a prolonged release dosage form with an improved bioavailability. Suitable carriers to sustain the release rate of a drug include, but are not limited to, ethyl cellulose, HPMC, HPMC-phtalate, colloidal silicondioxide and Eudragit-RSPM.

Pellets suitable to be used in the provided compositions and methods contain 50-80 % of a drug and 20-50% (w/w) of microcrystalline cellulose or other polymers. Suitable polymers include, but are not limited to, microcrystalline wax, pregelatinized starch and maltose dextrin.

Beads can be prepared in capsule and tablet dosage forms. Beads in tablet dosage form may demonstrate a slower dissolution profile than microparticles in capsule form. Microparticle fillers suitable for compositions and methods provided herein include, but not limited to, sorbitan monooleate (Span 80) and HPMC. Suitable dispersions for controlled release latex include, but not limited to, ethyl-acrylate and methyl-acrylate.

The pharmaceutical compositions provided herein may be provided in the form of microcapsules and microtablets. In one embodiment, microcapsules suitable for the compositions and methods provided herein comprise extended release polymer microcapsules containing aspirin and niacin with various solubility characteristics. Extended release polymer microcapsules can be prepared with colloidal polymer dispersion in an aqueous environment. In another embodiment, microcapsules suitable for the compositions and methods provided herein can be prepared using conventional microencapsulating techniques (Bodmeier & Wang, 1993).

Such multiparticulates may be made by the processes known to those skilled in the art, including wet-and dry-granulation, extrusion/spheronization, roller-compaction, melt-congealing, and by spray-coating seed cores. See, for example, Multiparticulate Oral Drug Delivery; Marcel Dekker: 1994; and Pharmaceutical Pelletization Technology; Marcel Dekker: 1989. Such materials used to form microparticulates are commercially available, for example, niacin is commercially available as Lonza niacin granular.

Other excipients as described herein may be blended with the pharmaceutical compositions to aid in processing and forming the multiparticulates. The resulting particles may themselves constitute the multiparticulate device or may be coated by various film-forming materials, such as enteric polymers, water-swellable, and water-soluble polymers. The multiparticulates can be further processed as a capsule or a tablet.

Additionally provided are pharmaceutical compositions in a dosage form that has an instant releasing component and at least one delayed releasing component, and is capable of giving a discontinuous release of the compound in the form of at least two consecutive pulses separated in time from 0.1 up to 24 hours.

Tablets-In-Capsule System

The pharmaceutical compositions provided herein may be provided in the form of tablets-in-capsule system, which is a multifunctional and multiple unit system comprising versatile mini-tablets in a hard gelatin capsule. It contains rapid-release mini-tablets, extended-release mini-tablets, pulsatile mini-tablets, and delayed-onset extended-release minitablets, each of which having specific lag times of release. Based on the combination of mini-tablets, multiplied pulsatile drug delivery system (DDS), site-specific DDS, slow-quick DDS, quick/slow DDS and zero-order DDS can be obtained.

Oral Administration

The pharmaceutical compositions provided herein may be provided in solid, semisolid, gelmatrix or liquid dosage forms for oral administration. As used herein, oral administration also include buccal, lingual, and sublingual administration. Suitable oral dosage forms include, but are not limited to, tablets, capsules, pills, troches, lozenges, pastilles, cachets, pellets, medicated chewing gum, granules, bulk powders, effervescent or non-effervescent powders or granules, solutions, emulsions, suspensions, solutions, wafers, sprinkles, elixirs, and syrups. In addition to the active ingredient(s), the pharmaceutical compositions may contain one or more pharmaceutically acceptable carriers or excipients, including, but not limited to, binders, fillers, diluents, disintegrants, wetting agents, lubricants, glidants, coloring agents, dye-migration inhibitors, sweetening agents, and flavoring agents.

Binders or granulators impart cohesiveness to a tablet to ensure the tablet remaining intact after compression. Suitable binders or granulators include, but are not limited to, starches, such as corn starch, potato starch, and pre-gelatinized starch (e.g., STARCH 1500); gelatin; sugars, such as sucrose, glucose, dextrose, molasses, and lactose; natural and synthetic gums, such as acacia, alginic acid, alginates, extract of Irish moss, Panwar gum, ghatti gum, mucilage of isabgol husks, carboxymethylcellulose, methylcellulose, polyvinylpyrrolidone (PVP), Veegum, larch arabogalactan, powdered tragacanth, and guar gum; celluloses, such as ethyl cellulose, cellulose acetate, carboxymethyl cellulose calcium, sodium carboxymethyl cellulose, methyl cellulose, hydroxyethylcellulose (HEC), hydroxypropylcellulose (HPC), hydroxypropyl methyl cellulose (HPMC); microcrystalline celluloses, such as AVICEL-PH-101, AVICEL-PH-103, AVICEL RC-581, AVICEL-PH-105 (FMC Corp., Marcus Hook, Pa.); and mixtures thereof. Suitable fillers include, but are not limited to, talc, calcium carbonate, microcrystalline cellulose, powdered cellulose, dextrates, kaolin, mannitol, silicic acid, sorbitol, starch, pre-gelatinized starch, and mixtures thereof. The binder or filler may be present from about 5 to about 49% by weight in the pharmaceutical compositions provided herein.

Suitable diluents include, but are not limited to, dicalcium phosphate, calcium sulfate, lactose, sorbitol, sucrose, inositol, cellulose, kaolin, mannitol, sodium chloride, dry starch, and powdered sugar. Certain diluents, such as mannitol, lactose, sorbitol, sucrose, and inositol, when present in sufficient quantity, can impart properties to some compressed tablets that permit disintegration in the mouth by chewing. Such compressed tablets can be used as chewable tablets.

Suitable disintegrants include, but are not limited to, agar; bentonite; celluloses, such as methylcellulose and carboxymethylcellulose; wood products; natural sponge; cation-exchange resins; alginic acid; gums, such as guar gum and Veegum HV; citrus pulp; cross-linked celluloses, such as croscarmellose; cross-linked polymers, such as crospovidone; cross-linked starches; calcium carbonate; microcrystalline cellulose, such as sodium starch glycolate; polacrilin potassium; starches, such as corn starch, potato starch, tapioca starch, and pre-gelatinized starch; clays; aligns; and mixtures thereof. The amount of disintegrant in the pharmaceutical compositions provided herein varies upon the type of formulation, and is readily discernible to those of ordinary skill in the art. The pharmaceutical compositions provided herein may contain from about 0.5 to about 15% or from about 1 to about 5% by weight of a disintegrant.

Suitable lubricants include, but are not limited to, calcium stearate; magnesium stearate; mineral oil; light mineral oil; glycerin; sorbitol; mannitol; glycols, such as glycerol behenate and polyethylene glycol (PEG); stearic acid; sodium lauryl sulfate; talc; hydrogenated vegetable oil, including peanut oil, cottonseed oil, sunflower oil, sesame oil, olive oil, corn oil, and soybean oil; zinc stearate; ethyl oleate; ethyl laureate; agar; starch; lycopodium; silica or silica gels, such as AEROSIL® 200 (W.R. Grace Co., Baltimore, Md.) and CAB-O-SIL® (Cabot Co. of Boston, Mass.); and mixtures thereof. The pharmaceutical compositions provided herein may contain about 0.1 to about 5% by weight of a lubricant.

Suitable glidants include colloidal silicon dioxide, CAB-O-SIL® (Cabot Co. of Boston, Mass.), and asbestos-free talc. Coloring agents include any of the approved, certified, water soluble FD&C dyes, and water insoluble FD&C dyes suspended on alumina hydrate, and color lakes and mixtures thereof. A color lake is the combination by adsorption of a water-soluble dye to a hydrous oxide of a heavy metal, resulting in an insoluble form of the dye. Flavoring agents include natural flavors extracted from plants, such as fruits, and synthetic blends of compounds which produce a pleasant taste sensation, such as peppermint and methyl salicylate. Sweetening agents include sucrose, lactose, mannitol, syrups, glycerin, sucralose, and artificial sweeteners, such as saccharin and aspartame. Suitable emulsifying agents include gelatin, acacia, tragacanth, bentonite, and surfactants, such as polyoxyethylene sorbitan monooleate (TWEEN® 20), polyoxyethylene sorbitan monooleate 80 (TWEEN 80), and triethanolamine oleate. Suspending and dispersing agents include sodium carboxymethylcellulose, pectin, tragacanth, Veegum, acacia, sodium carbomethylcellulose, hydroxypropyl methylcellulose, and polyvinylpyrolidone. Preservatives include glycerin, methyl and propylparaben, benzoic add, sodium benzoate and alcohol. Wetting agents include propylene glycol monostearate, sorbitan monooleate, diethylene glycol monolaurate, and polyoxyethylene lauryl ether. Solvents include glycerin, sorbitol, ethyl alcohol, and syrup. Examples of non-aqueous liquids utilized in emulsions include mineral oil and cottonseed oil. Organic acids include citric and tartaric acid. Sources of carbon dioxide include sodium bicarbonate and sodium carbonate.

It should be understand that many carriers and excipients may serve several functions, even within the same formulation.

The pharmaceutical compositions provided herein may be provided as compressed tablets, tablet triturates, chewable lozenges, rapidly dissolving tablets, multiple compressed tablets, or enteric-coating tablets, sugar-coated, or film-coated tablets. Enteric-coated tablets are compressed tablets coated with substances that resist the action of stomach acid but dissolve or disintegrate in the intestine, thus protecting the active ingredients from the acidic environment of the stomach. Enteric-coatings include, but are not limited to, fatty acids, fats, phenylsalicylate, waxes, shellac, ammoniated shellac, and cellulose acetate phthalates. Sugar-coated tablets are compressed tablets surrounded by a sugar coating, which may be beneficial in covering up objectionable tastes or odors and in protecting the tablets from oxidation. Film-coated tablets are compressed tablets that are covered with a thin layer or film of a water-soluble material. Film coatings include, but are not limited to, hydroxyethylcellulose, sodium carboxymethylcellulose, polyethylene glycol 4000, and cellulose acetate phthalate. Film coating imparts the same general characteristics as sugar coating. Multiple compressed tablets are compressed tablets made by more than one compression cycle, including layered tablets, and press-coated or dry-coated tablets.

The tablet dosage forms may be prepared from the active ingredient in powdered, crystalline, or granular forms, alone or in combination with one or more carriers or excipients described herein, including binders, disintegrants, controlled-release polymers, lubricants, diluents, and/or colorants. Flavoring and sweetening agents are especially useful in the formation of chewable tablets and lozenges.

The pharmaceutical compositions provided herein may be provided as soft or hard capsules, which can be made from gelatin, methylcellulose, starch, or calcium alginate. The hard gelatin capsule, also known as the dry-filled capsule (DFC), consists of two sections, one slipping over the other, thus completely enclosing the active ingredient. The soft elastic capsule (SEC) is a soft, globular shell, such as a gelatin shell, which is plasticized by the addition of glycerin, sorbitol, or a similar polyol. The soft gelatin shells may contain a preservative to prevent the growth of microorganisms. Suitable preservatives are those as described herein, including methyl- and propyl-parabens, and sorbic acid. The liquid, semisolid, and solid dosage forms provided herein may be encapsulated in a capsule. Suitable liquid and semisolid dosage forms include solutions and suspensions in propylene carbonate, vegetable oils, or triglycerides. Capsules containing such solutions can be prepared as described in U.S. Pat. Nos. 4,328,245; 4,409,239; and 4,410,545. The capsules may also be coated as known by those of skill in the art in order to modify or sustain dissolution of the active ingredient.

The pharmaceutical compositions provided herein may be provided in liquid and semisolid dosage forms, including emulsions, solutions, suspensions, elixirs, and syrups. An emulsion is a two-phase system, in which one liquid is dispersed in the form of small globules throughout another liquid, which can be oil-in-water or water-in-oil. Emulsions may include a pharmaceutically acceptable non-aqueous liquids or solvent, emulsifying agent, and preservative. Suspensions may include a pharmaceutically acceptable suspending agent and preservative. Aqueous alcoholic solutions may include a pharmaceutically acceptable acetal, such as a di(lower alkyl)acetal of a lower alkyl aldehyde (the term “lower” means an alkyl having between 1 and 6 carbon atoms), e.g., acetaldehyde diethyl acetal; and a water-miscible solvent having one or more hydroxyl groups, such as propylene glycol and ethanol. Elixirs are clear, sweetened, and hydroalcoholic solutions. Syrups are concentrated aqueous solutions of a sugar, for example, sucrose, and may also contain a preservative. For a liquid dosage form, for example, a solution in a polyethylene glycol may be diluted with a sufficient quantity of a pharmaceutically acceptable liquid carrier, e.g., water, to be measured conveniently for administration.

The pharmaceutical compositions provided herein for oral administration may be also provided in the forms of liposomes, micelles, microspheres, or nanosystems. Miccellar dosage forms can be prepared as described in U.S. Pat. No. 6,350,458.

The pharmaceutical compositions provided herein may be provided as non-effervescent or effervescent, granules and powders, to be reconstituted into a liquid dosage form. Pharmaceutically acceptable carriers and excipients used in the non-effervescent granules or powders may include diluents, sweeteners, and wetting agents. Pharmaceutically acceptable carriers and excipients used in the effervescent granules or powders may include organic acids and a source of carbon dioxide.

Coloring and flavoring agents can be used in all of the above dosage forms.

The pharmaceutical compositions provided herein may be formulated as immediate or modified release dosage forms, including delayed-, extended, pulsed-, controlled, targeted-, and programmed-release forms.

The pharmaceutical compositions provided herein may be co-formulated with other active ingredients which do not impair the desired therapeutic action, or with substances that supplement the desired action.

Methods

In one embodiment, provided herein are methods for reducing niacin-induced flushing in a subject comprising administering to a subject a formulation comprising niacin and a flush-inhibiting regimen of aspirin in amounts which are effective to reduce the flushing (including burning, itching, crawling sensation, pain, reddening of the skin, and/or fever like sensation).

In one embodiment, the methods provided herein utilize aspirin and nicotinic acid. In another embodiment, In another embodiment, the methods provided herein utilize aspirin and nicotinic acid metabolites.

In one embodiment, provided are methods for reducing niacin-induced flushing comprising administering to a subject a pharmaceutical composition comprising a niacin/aspirin dosing regimen comprising a total daily dose of aspirin of about 80 to about 2000 mg. In another embodiment, the total daily dose of aspirin of about 80 to about 500 mg. In another embodiment, the total daily dose of aspirin of about 80 to about 400 mg.

In one embodiment, the methods provided herein comprise niacin/aspirin pharmaceutical composition wherein the total aspirin daily dose is released from the composition based on an aspirin release profile, wherein 80% of aspirin is released over a period of time of about 2 to about 16 hours following administration of the composition. In another embodiment, the total daily dose of aspirin is released from the composition based on an aspirin release profile, wherein 90% of aspirin is released over a period of time of about 2 to about 16 hours following administration of the composition. In yet another embodiment, the total daily dose of aspirin is released from the composition based on an aspirin release profile, wherein aspirin concentration in plasma is greater than 5% of Cmax over a period of time of about 2 to about 16 hours following administration of the composition. In still another embodiment, the total daily dose of aspirin is released from the composition based on an aspirin release profile, wherein aspirin concentration in plasma is greater than 10% of Cmax over a period of time of about 2 to about 16 hours following administration of the composition.

In one embodiment, the methods provided herein comprise niacin/aspirin pharmaceutical composition wherein the total aspirin daily dose is released from the composition based on an aspirin release profile, wherein 80% of aspirin is released over a period of time of about 2 to about 8 hours following administration of the composition. In another embodiment, the total daily dose of aspirin is released from the composition based on an aspirin release profile, wherein 90% of aspirin is released over a period of time of about 2 to about 8 hours following administration of the composition. In yet another embodiment, the total daily dose of aspirin is released from the composition based on an aspirin release profile, wherein aspirin concentration in plasma is greater than 5% of Cmax over a period of time of about 2 to about 8 hours following administration of the composition. In still another embodiment, the total daily dose of aspirin is released from the composition based on an aspirin release profile, wherein aspirin concentration in plasma is greater than 10% of Cmax over a period of time of about 2 to about 8 hours following administration of the composition. In one embodiment, the methods provided herein comprise niacin/aspirin pharmaceutical composition wherein the total aspirin daily dose is released from the composition based on an aspirin release profile, wherein 80% of aspirin is released over a period of time of about 2 to about 6 hours following administration of the composition. In another embodiment, the total daily dose of aspirin is released from the composition based on an aspirin release profile, wherein 90% of aspirin is released over a period of time of about 2 to about 6 hours following administration of the composition. In yet another embodiment, the total daily dose of aspirin is released from the composition based on an aspirin release profile, wherein aspirin concentration in plasma is greater than 5% of Cmax over a period of time of about 2 to about 6 hours following administration of the composition. In still another embodiment, the total daily dose of aspirin is released from the composition based on an aspirin release profile, wherein aspirin concentration in plasma is greater than 10% of Cmax over a period of time of about 2 to about 6 hours following administration of the composition. In one embodiment, the methods provided herein comprise niacin/aspirin pharmaceutical composition wherein the total aspirin daily dose is released from the composition based on an aspirin release profile, wherein 80% of aspirin is released over a period of time of about 3 to about 4 hours following administration of the composition. In another embodiment, the total daily dose of aspirin is released from the composition based on an aspirin release profile, wherein 90% of aspirin is released over a period of time of about 3 to about 4 hours following administration of the composition. In yet another embodiment, the total daily dose of aspirin is released from the composition based on an aspirin release profile, wherein aspirin concentration in plasma is greater than 10% of Cmax over a period of time of about 3 to about 4 hours following administration of the composition. In still another embodiment, the total daily dose of aspirin is released from the composition based on an aspirin release profile, wherein aspirin concentration in plasma is greater than 20% of Cmax over a period of time of about 3 to about 4 hours following administration of the composition.

In one embodiment, the methods provided herein comprise niacin/aspirin pharmaceutical composition wherein the total aspirin daily dose is released from the composition based on an aspirin release profile, wherein 80% of aspirin is released over a period of time of about 2 to about 10 hours following administration of the composition. In another embodiment, the total daily dose of aspirin is released from the composition based on an aspirin release profile, wherein 90% of aspirin is released over a period of time of about 2 to about 10 hours following administration of the composition. In yet another embodiment, the total daily dose of aspirin is released from the composition based on an aspirin release profile, wherein aspirin concentration in plasma is greater than 5% of Cmax over a period of time of about 2 to about 10 hours following administration of the composition. In still another embodiment, the total daily dose of aspirin is released from the composition based on an aspirin release profile, wherein aspirin concentration in plasma is greater than 10% of Cmax over a period of time of about 2 to about 10 hours following administration of the composition. In one embodiment, the methods provided herein comprise niacin/aspirin pharmaceutical composition wherein the total aspirin daily dose is released from the composition based on an aspirin release profile, wherein 80% of aspirin is released over a period of time of about 3 to about 8 hours following administration of the composition. In another embodiment, the total daily dose of aspirin is released from the composition based on an aspirin release profile, wherein 90% of aspirin is released over a period of time of about 3 to about 8 hours following administration of the composition. In yet another embodiment, the total daily dose of aspirin is released from the composition based on an aspirin release profile, wherein aspirin concentration in plasma is greater than 5% of Cmax over a period of time of about 3 to about 8 hours following administration of the composition. In still another embodiment, the total daily dose of aspirin is released from the composition based on an aspirin release profile, wherein aspirin concentration in plasma is greater than 10% of Cmax over a period of time of about 3 to about 8 hours following administration of the composition. In one embodiment, the methods provided herein comprise niacin/aspirin pharmaceutical composition wherein the total aspirin daily dose is released from the composition based on an aspirin release profile, wherein 80% of aspirin is released over a period of time of about 4 to about 6 hours following administration of the composition. In another embodiment, the total daily dose of aspirin is released from the composition based on an aspirin release profile, wherein 90% of aspirin is released over a period of time of about 4 to about 6 hours following administration of the composition. In yet another embodiment, the total daily dose of aspirin is released from the composition based on an aspirin release profile, wherein aspirin concentration in plasma is greater than 5% of Cmax over a period of time of about 4 to about 6 hours following administration of the composition. In still another embodiment, the total daily dose of aspirin is released from the composition based on an aspirin release profile, wherein aspirin concentration in plasma is greater than 10% of Cmax over a period of time of about 4 to about 6 hours following administration of the composition.

In one embodiment, the methods provided herein comprise niacin/aspirin pharmaceutical composition wherein the total aspirin daily dose is released from the composition based on an aspirin release profile, wherein 80% of aspirin is released over a period of time of about 3 to about 12 hours following administration of the composition. In another embodiment, the total daily dose of aspirin is released from the composition based on an aspirin release profile, wherein 90% of aspirin is released over a period of time of about 3 to about 12 hours following administration of the composition. In yet another embodiment, the total daily dose of aspirin is released from the composition based on aspirin release profile, wherein aspirin concentration in plasma is greater than 5% of Cmax over a period of time of about 3 to about 12 hours following administration of the composition. In still another embodiment, the total daily dose of aspirin is released from the composition based on an aspirin release profile, wherein aspirin concentration in plasma is greater than 10% of Cmax over a period of time of about 3 to about 12 hours following administration of the composition. In one embodiment, the methods provided herein comprise niacin/aspirin pharmaceutical composition wherein the total aspirin daily dose is released from the composition based on an aspirin release profile, wherein 80% of aspirin is released over a period of time of about 4 to about 8 hours following administration of the composition. In another embodiment, the total daily dose of aspirin is released from the composition based on an aspirin release profile, wherein 90% of aspirin AUC is released over a period of time of about 4 to about 8 hours following administration of the composition. In yet another embodiment, the total daily dose of aspirin is released from the composition based on an aspirin release profile, wherein aspirin concentration in plasma is greater than 5% of Cmax over a period of time of about 4 to about 8 hours following administration of the composition. In still another embodiment, the total daily dose of aspirin is released from the composition based on an aspirin release profile, wherein aspirin concentration in plasma is greater than 10% of Cmax over a period of time of about 4 to about 8 hours following administration of the composition. In one embodiment, the methods provided herein comprise niacin/aspirin pharmaceutical composition wherein the total aspirin daily dose is released from the composition based on an aspirin release profile, wherein 80% of aspirin AUC is released over a period of time of about 3 to about 6 hours following administration of the composition. In another embodiment, the total daily dose of aspirin is released from the composition based on an aspirin release profile, wherein 90% of aspirin is released over a period of time of about 3 to about 6 hours following administration of the composition. In yet another embodiment, the total daily dose of aspirin is released from the composition based on an aspirin release profile, wherein aspirin concentration in plasma is greater than 5% of Cmax over a period of time of about 3 to about 6 hours following administration of the composition. In still another embodiment, the total daily dose of aspirin is released from the composition based on an aspirin release profile, wherein aspirin concentration in plasma is greater than 10% of Cmax over a period of time of about 3 to about 6 hours following administration of the composition.

In one embodiment, the methods provided herein comprise niacin/aspirin pharmaceutical composition wherein the total aspirin daily dose is released from the composition based on an aspirin release profile, wherein 80% of aspirin is released over a period of time of about 4 to about 16 hours following administration of the composition. In another embodiment, the total daily dose of aspirin is released from the composition based on an aspirin release profile, wherein 90% of aspirin is released over a period of time of about 4 to about 16 hours following administration of the composition. In yet another embodiment, the total daily dose of aspirin is released from the composition based on an aspirin release profile, wherein aspirin concentration in plasma is greater than 5% of Cmax over a period of time of about 4 to about 16 hours following administration of the composition. In still another embodiment, the total daily dose of aspirin is released from the composition based on an aspirin release profile, wherein aspirin concentration in plasma is greater than 10% of Cmax over a period of time of about 4 to about 16 hours following administration of the composition. In one embodiment, the methods provided herein comprise niacin/aspirin pharmaceutical composition wherein the total aspirin daily dose is released from the composition based on an aspirin release profile, wherein 80% of aspirin is released over a period of time of about 5 to about 12 hours following administration of the composition. In another embodiment, the total daily dose of aspirin is released from the composition based on an aspirin release profile, wherein 90% of aspirin is released over a period of time of about 5 to about 12 hours following administration of the composition. In yet another embodiment, the total daily dose of aspirin is released from the composition based on an aspirin release profile, wherein aspirin concentration in plasma is greater than 5% of Cmax over a period of time of about 5 to about 12 hours following administration of the composition. In still another embodiment, the total daily dose of aspirin is released from the composition based on an aspirin release profile, wherein aspirin concentration in plasma is greater than 10% of Cmax over a period of time of about 5 to about 12 hours following administration of the composition. In one embodiment, the methods provided herein comprise niacin/aspirin pharmaceutical composition wherein the total aspirin daily dose is released from the composition based on an aspirin release profile, wherein 80% of aspirin is released over a period of time of about 6 to about 10 hours following administration of the composition. In another embodiment, the total daily dose of aspirin is released from the composition based on an aspirin release profile, wherein 90% of aspirin is released over a period of time of about 6 to about 10 hours following administration of the composition. In yet another embodiment, the total daily dose of aspirin is released from the composition based on an aspirin release profile, wherein aspirin concentration in plasma is greater than 5% of Cmax over a period of time of about 6 to about 10 hours following administration of the composition. In still another embodiment, the total daily dose of aspirin is released from the composition based on an aspirin release profile, wherein aspirin concentration in plasma is greater than 10% of Cmax over a period of time of about 6 to about 10 hours following administration of the composition.

In another embodiment, the methods provided herein comprise a step of predosing a subject with an aspirin regimen on the day of niacin therapy, wherein about 80% of niacin AUC is not released until after about 16 hours of predosing with aspirin. In another embodiment, about 90% of niacin AUC is not released until after about 16 hours of predosing with aspirin. In another embodiment, the plasma concentration of niacin is less than 20% of Cmax until after about 16 hours of predosing with aspirin. In yet another embodiment, the plasma concentration of niacin is less than 10% of Cmax until after about 16 hours of predosing with aspirin.

In another embodiment, the methods provided herein comprise a step of predosing a subject with an aspirin regimen on the day of niacin therapy, wherein about 80% of niacin AUC is not released until after about 14 hours of predosing with aspirin. In another embodiment, about 90% of niacin AUC is not released until after about 14 hours of predosing with aspirin. In another embodiment, the plasma concentration of niacin is less than 20% of Cmax until after about 14 hours of predosing with aspirin. In yet another embodiment, the plasma concentration of niacin is less than 10% of Cmax until after about 14 hours of predosing with aspirin.

In another embodiment, the methods provided herein comprise a step of predosing a subject with an aspirin regimen on the day of niacin therapy, wherein about 80% of niacin AUC is not released until after about 12 hours of predosing with aspirin. In another embodiment, about 90% of niacin AUC is not released until after about 12 hours of predosing with aspirin. In another embodiment, the plasma concentration of niacin is less than 20% of Cmax until after about 12 hours of predosing with aspirin. In yet another embodiment, the plasma concentration of niacin is less than 10% of Cmax until after about 12 hours of predosing with aspirin.

In another embodiment, the methods provided herein comprise a step of predosing a subject with an aspirin regimen on the day of niacin therapy, wherein about 80% of niacin AUC is not released until after about 10 hours of predosing with aspirin. In another embodiment, about 90% of niacin AUC is not released until after about 10 hours of predosing with aspirin. In another embodiment, the plasma concentration of niacin is less than 20% of Cmax until after about 10 hours of predosing with aspirin. In yet another embodiment, the plasma concentration of niacin is less than 10% of Cmax until after about 10 hours of predosing with aspirin.

In another embodiment, the methods provided herein comprise a step of predosing a subject with an aspirin regimen on the day of niacin therapy, wherein about 80% of niacin AUC is not released until after about 8 hours of predosing with aspirin. In another embodiment, about 90% of niacin AUC is not released until after about 8 hours of predosing with aspirin. In another embodiment, the plasma concentration of niacin is less than 20% of Cmax until after about 8 hours of predosing with aspirin. In yet another embodiment, the plasma concentration of niacin is less than 10% of Cmax until after about 8 hours of predosing with aspirin.

In another embodiment, the methods provided herein comprise a step of predosing a subject with an aspirin regimen on the day of niacin therapy, wherein about 80% of niacin AUC is not released until after about 6.5 hours of predosing with aspirin. In another embodiment, about 90% of niacin AUC is not released until after about 6.5 hours of predosing with aspirin. In another embodiment, the plasma concentration of niacin is less than 20% of Cmax until after about 6.5 hours of predosing with aspirin. In yet another embodiment, the plasma concentration of niacin is less than 10% of Cmax until after about 6.5 hours of predosing with aspirin.

In another embodiment, the methods provided herein comprise a step of predosing a subject with an aspirin regimen on the day of niacin therapy, wherein about 80% of niacin AUC is not released until after about 6 hours of predosing with aspirin. In another embodiment, about 90% of niacin AUC is not released until after about 6 hours of predosing with aspirin. In another embodiment, the plasma concentration of niacin is less than 20% of Cmax until after about 6 hours of predosing with aspirin. In yet another embodiment, the plasma concentration of niacin is less than 10% of Cmax until after about 6 hours of predosing with aspirin.

In another embodiment, the methods provided herein comprise a step of predosing a subject with an aspirin regimen on the day of niacin therapy, wherein about 80% of niacin AUC is not released until after about 5 hours of predosing with aspirin. In another embodiment, about 90% of niacin AUC is not released until after about 5 hours of predosing with aspirin. In another embodiment, the plasma concentration of niacin is less than 20% of Cmax until after about 5 hours of predosing with aspirin. In yet another embodiment, the plasma concentration of niacin is less than 10% of Cmax until after about 5 hours of predosing with aspirin.

In another embodiment, the methods provided herein comprise a step of predosing a subject with an aspirin regimen on the day of niacin therapy, wherein about 80% of niacin AUC is not released until afetr about 4 hours of predosing with aspirin. In another embodiment, about 90% of niacin AUC is not released until after about 4 hours of predosing with aspirin. In another embodiment, the plasma concentration of niacin is less than 20% of Cmax until after about 4 hours of predosing with aspirin. In yet another embodiment, the plasma concentration of niacin is less than 10% of Cmax until after about 4 hours of predosing with aspirin.

In another embodiment, the methods provided herein comprise a step of predosing a subject with an aspirin regimen on the day of niacin therapy, wherein about 80% of niacin AUC is not released until after about 3 hours of predosing with aspirin. In another embodiment, about 90% of niacin AUC is not released until after about 3 hours of predosing with aspirin. In another embodiment, the plasma concentration of niacin is less than 20% of Cmax until after about 3 hours of predosing with aspirin. In yet another embodiment, the plasma concentration of niacin is less than 10% of Cmax until after about 3 hours of predosing with aspirin.

In another embodiment, the methods provided herein comprise a step of predosing a subject with aspirin regimen on the day of niacin therapy, wherein about 80% of niacin AUC is not released until after about 2 hours of predosing with aspirin. In another embodiment, about 90% of niacin AUC is not released until after about 2 hours of predosing with aspirin. In another embodiment, the plasma concentration of niacin is less than 20% of Cmax until after about 2 hours of predosing with aspirin. In yet another embodiment, the plasma concentration of niacin is less than 10% of Cmax until after about 2 hours of predosing with aspirin.

In another embodiment, the methods provided herein comprise a step of predosing a subject with aspirin regimen on the day of niacin therapy, wherein about 80% of niacin AUC is not released until after about 1 hour of predosing with aspirin. In another embodiment, about 90% of niacin AUC is not released until after about 1 hour of predosing with aspirin. In another embodiment, the plasma concentration of niacin is less than 20% of Cmax until after about 1 hour of predosing with aspirin. In yet another embodiment, the plasma concentration of niacin is less than 10% of Cmax until after about 1 hour of predosing with aspirin.

In another embodiment, the methods provided herein comprise a step of predosing a subject with aspirin for 1-7 days before initiation of niacin therapy. Such pretreatment may use an immediate or extended release aspirin formulation.

In another embodiment, the methods provided herein comprise niacin/aspirin dosing regimens that reduce niacin-induced flushing, further comprising a lipid-lowering drug other than niacin. In another embodiment, the methods provided herein comprise niacin and aspirin dosing regimens further comprising anti-obesity agents, anti-diabetic agents and anti-hypertensive agents.

In another embodiment, provided are methods for reducing at least one component of the flushing symptoms comprising administration to a subject a niacin/aspirin formulation. These symptoms include, but not limited to, redness, warmth, tingling, itching, burning, fever-like sensation and crawling sensation of the skin.

In another embodiment, provided are methods for decreasing prostaglandin-related side effects comprising administering to a patient a niacin/aspirin formulation.

In another embodiment, provided are methods for decreased discontinuation of niacin treatment comprising administering to a patient a niacin/aspirin formulation. In yet another embodiment, provided are methods for increased patient compliance with niacin treatment comprising administering to a patient a niacin/aspirin formulation.

In another embodiment, provided are methods for prevention of aspirin hydrolysis prior to its release from an aspirin/niacin formulation comprising administering to a patient the aspirin/niacin formulation, wherein aspirin microparticles and niacin microparticles have different release profiles.

In another aspect, the methods provided herein can be used to treat atherosclerosis, cardiovascular diseases, dyslipidemias, and metabolic diseases, including, but not limited to diabetes, obesity, metabolic syndrome, and genetic abnormalities associated with increased cardiovascular risk.

The dosage regimen suitable for the methods provided herein can include prolonged multi-day dosing of specific doses of aspirin with a regimen which accumulates an aspirin inhibitory effect to an effective amount before the niacin level reaches a flush-inducing level, or it can be a regimen which produces the effective level within a short time.

The methods involve pretreatment of a subject with aspirin in an amount sufficient to inhibit synthesis of prostaglandin D2 (PGD2). In one embodiment, the pretreatment is continued for a period of 1-4 days prior to administration of niacin. In another embodiment, the pretreatment is continued for a period of 2-4 days prior to administration of niacin. In yet another embodiment, the pretreatment is continued for a period of 3-4 days prior to the initial niacin dose.

During pretreatment, aspirin is administered in at least one dose daily. In one embodiment, aspirin is administered in two or more doses daily. A extended release dosage form may be administered fewer times daily than a comparable immediate release form, while providing similar protective serum concentrations of aspirin.

The methods further provide for continued administration of aspirin while niacin is being administered. The niacin may be administered initially at a dosage level which is sufficient to produce lipid lowering effects in the subject, or may by administered initially at a lower level and raised progressively to lipid lowering dosage levels. The daily dosage of niacin may be taken at one time or be divided into multiple doses taken, for example, 2-4 times per day. Each dose could be multiple capsules or tablets containing a dosage form niacin and aspirin.

On one embodiment, niacin can be released as immediate release niacin, extended release niacin, or biphasic or triphasic release of immediate release niacin. In certain embodiments, one pill could be formulated to contain an amount of niacin to be released immediately after administration. In another embodiment, one pill could be formulated to contain an amount of niacin to be released about 2 hours after administration. In yet another embodiment, one pill could be formulated to contain an amount of niacin to be released about 3 hours after administration. In another embodiment, one pill could be formulated to contain an amount of niacin to be released about 4 hours after administration. In another embodiment, one pill could be formulated to contain an amount of niacin to be released about 5 hours after administration. In another embodiment, one pill could be formulated to contain an amount of niacin to be released about 6 hours after administration. In another embodiment, one pill could be formulated to contain an amount of niacin to be released about 6.5 hours after administration.

Although predosing with a extended release aspirin can be conducted as long as desired, for convenience, in one embodiment, aspirin is administered up to 7 days prior to administering niacin. In another embodiment, aspirin is administered up to 4 days prior to administering niacin. In another embodiment, aspirin is administered up to 3 days prior to administering niacin. In yet another embodiment, aspirin is administered up to 2 days prior to administering niacin. In yet another embodiment, aspirin is administered up to 1 day prior to administering niacin. In another embodiment, aspirin is administered up to 24 hours prior to administering niacin. In yet another embodiment, aspirin is administered up to 16 hours prior to administering niacin. In another embodiment, aspirin is administered up to 14 hours prior to administering niacin. In another embodiment, aspirin is administered up to 12 hours prior to administering niacin. In another embodiment, aspirin is administered up to 10 hours prior to administering niacin. In another embodiment, aspirin is administered up to 8 hours prior to administering niacin. In another embodiment, aspirin is administered up to 6 hours prior to administering niacin. In yet another embodiment, aspirin is administered up to 4 hours prior to administering niacin. In another embodiment, aspirin is administered up to 3 hours prior to administering niacin. In another embodiment, aspirin is administered up to 2 hours prior to administering niacin. In yet another embodiment, aspirin is administered up to 1 hour prior to administering niacin.

In one embodiment, methods for reducing niacin-induced flushing comprising administering to a subject a pharmaceutical composition comprising a niacin/aspirin dosing regimen comprising a total daily dose of aspirin of about 80 to about 2000 mg. In another embodiment, the total daily dose of aspirin of about 80 to about 500 mg. In another embodiment, the total daily dose of aspirin of about 80 to about 400 mg.

In one embodiment, the total aspirin daily dose is about 80 mg that is released over a 4 hour period. In another embodiment, the total aspirin daily dose is about 80 mg that is released over a 3 hour period. In yet another embodiment, the total aspirin daily dose is about 80 mg that is released over a 2 hour period. In another embodiment, the total aspirin daily dose is about 80 mg that is released over a 1 hour period. In another embodiment, a subject is predosed on the day of niacin therapy with a similar aspirin regimen initiated about 2 to about 5 hours before niacin therapy. In another embodiment, the total aspirin daily dose is about 81 mg.

In one embodiment, the total aspirin daily dose is about 120 mg that is released over a 6 hour period. In another embodiment, the total aspirin daily dose is about 120 mg that is released over a 4 hour period. In yet another embodiment, the total aspirin daily dose is about 120 mg that is released over a 3 hour period. In another embodiment, the total aspirin daily dose is about 120 mg that is released over a 2 hour period. In another embodiment, a subject is predosed on the day of niacin therapy with a similar aspirin regimen initiated about 2 to about 8 hours before niacin therapy.

In one embodiment, the total aspirin daily dose is about 160 mg that is released over a 8 hour period. In another embodiment, the total aspirin daily dose is about 160 mg that is released over a 5-6 hour period. In yet another embodiment, the total aspirin daily dose is about 160 mg that is released over a 4 hour period. In another embodiment, the total aspirin daily dose is about 160 mg that is released over a 2-3 hour period. In another embodiment, a subject is predosed on the day of niacin therapy with a similar aspirin regimen initiated about 2 to about 11 hours before niacin therapy. In another embodiment, the total aspirin daily dose is about 162 mg.

In one embodiment, the total aspirin daily dose is about 240 mg that is released over a 12 hour period. In another embodiment, the total aspirin daily dose is about 240 mg that is released over a 8 hour period. In yet another embodiment, the total aspirin daily dose is about 240 mg that is released over a 6 hour period. In another embodiment, the total aspirin daily dose is about 240 mg that is released over a 4 hour period. In another embodiment, a subject is predosed on the day of niacin therapy with a similar aspirin regimen initiated about 2 to about 16 hours before niacin therapy. In another embodiment, the total aspirin daily dose is about 243 mg.

In one embodiment, the total aspirin daily dose is about 320 mg that is released over a 16 hour period. In another embodiment, the total aspirin daily dose is about 320 mg that is released over a 10-11 hour period. In yet another embodiment, the total aspirin daily dose is about 320 mg that is released over a 8 hour period. In another embodiment, the total aspirin daily dose is about 320 mg that is released over a 5-6 hour period. In another embodiment, the total aspirin daily dose is about 320 mg that is released over a 4 hour period. In another embodiment, a subject is predosed on the day of niacin therapy with a similar aspirin regimen initiated about 2 to about 16 hours before niacin therapy. In another embodiment, the total aspirin daily dose is about 324 mg.

In another embodiment, the total daily dose includes 1-7 days of aspirin pretreatment. In yet another embodiment, aspirin dosing includes 2-4 days of aspirin pretreatment. Such pretreatment may be an immediate or extended release aspirin formulation.

In one embodiment, the niacin-aspirin combination, for example, may be a bilayer tablet where one of the layers is extended release aspirin, and the other layer is an immediate release niacin. In another embodiment, the niacin-aspirin combination may be a bilayer tablet where the inner layer is a delayed-release coated immediate release or extended release niacin, and the outer layer is aspirin, and the other layer is an immediate release or extended release aspirin.

In an alternative embodiment, niacin-aspirin formulation may comprise two tablets, where one tablet is a delayed-release coated immediate release or extended release niacin, and the other tablet contains immediate release or extended release aspirin. In another embodiment, a lipid-lowering drug other than niacin, is optionally present.

In another embodiment, a subject is given a predose of aspirin that includes a major proportion of a extended release aspirin formulated based on the methods provided herein and a minor proportion of an immediate release aspirin, which can be in the conventional form. The immediate release form quickly raises the level of aspirin to an effective levels and the extended release portion maintains the effective level. With this combination, the predosing period is reduced. For example, the immediate release portion of aspirin can be from 20 to 80 mg per unit dose and the extended release dosage formulation can be in the amount of about 80 to about 400 mg released over about 2 to about 16 hours.

In one embodiment, provided herein are methods for reducing niacin-induced flushing in a subject comprising administering to a subject a formulation comprising niacin and a flush-inhibiting regimen of ibuprofen in amounts which are effective to reduce the flushing (including burning, itching, crawling sensation, pain, reddening of the skin, and/or fever like sensation), wherein the total ibuprofen daily dose is about 120 mg to about 325 mg.

In another embodiment, provided herein are methods for reducing niacin-induced flushing in a subject comprising administering to a subject a formulation comprising niacin and a flush-inhibiting regimen of indomethacin in amounts which are effective to reduce the flushing (including burning, itching, crawling sensation, pain, reddening of the skin, and/or fever like sensation), wherein the total indomethacin daily dose is about 25 mg to about 30 mg.

In yet another embodiment, provided herein are methods for reducing niacin-induced flushing in a subject comprising administering to a subject a formulation comprising niacin and a flush-inhibiting regimen of phenylbutazone in amounts which are effective to reduce the flushing (including burning, itching, crawling sensation, pain, reddening of the skin, and/or fever like sensation), wherein the total phenylbutazone daily dose is about 150 mg to about 200 mg.

In another embodiment, provided herein are methods for reducing niacin-induced flushing in a subject comprising administering to a subject a formulation comprising niacin and a flush-inhibiting regimen of naproxen in amounts which are effective to reduce the flushing (including burning, itching, crawling sensation, pain, reddening of the skin, and/or fever like sensation), wherein the total naproxen daily dose is about 150 mg to about 200 mg.

In another embodiment, provided herein are methods for treating or preventing diseases and disorders including, but not limited to, (i) disorders of lipoprotein metabolism including but not limited to, dyslipidemia, dyslipoproteinemia, lipoprotein overproduction or deficiency, elevation of total cholesterol levels, elevation of low density lipoprotein concentration, elevation of triglyceride concentration, lipid elimination in bile, metabolic disorder, phospholipid elimination in bile, oxysterol elimination in bile, abnormal bile production, and peroxisome proliferator activated receptor-associated disorders; (ii) disorders of glucose metabolism including, but not limited to, insulin resistance, impaired glucose tolerance, impaired fasting glucose levels in blood, diabetes mellitus, lipodystrophy, central obesity, peripheral lipoatrophy, diabetic nephropathy, diabetic retinopathy, renal disease, and septicemia; (iii) cardiovascular disorders and related vascular disorders including, but not limited to, atherosclerosis, hypertension, coronary artery disease, myocardial infarction, arrhythmia, atrial fibrillation, heart valve disease, heart failure, cardiomyopathy, myopathy, pericarditis, impotence and thrombotic disorders; (iv) modulating inflammation markers and/or C-reactive protein and related disorders including, but not limited to, inflammation, ischemic necrosis, colon cancer, thrombotic disorders; and (v) aging, Alzheimer's disease, Parkinson's disease, pancreatitis, and pancreatitius.

Provided below are non-limiting examples.

### Examples

### Example 1

Provided is the clinical study to establish an anti-flushing effect of aspirin regimen on immediate release niacin.

The product is a niacin/aspirin oral dosage form.

Trial Design

The primary endpoints for this study were treatment group comparisons of:
incidence, duration and severity of flushing following niacin dosing;
routine monitoring adverse events.

Subjects have screening procedures performed up to 4 weeks prior to the first dosing visit. All qualified subjects received two single doses of niacin one week apart. Subjects were randomized to receive either an aspirin regimen or a placebo regimen prior to their niacin dose on day 1 and the opposite regimen prior to their niacin dose on day 8. At randomization, subjects received blinded study medication to be taken in the morning and evening of days −4, −3, −2 and −1. Subjects checked into the clinic on day −1 and remained housed for approximately 24 hours after niacin dosing on day 1. Prior to leaving the clinic, they received blinded study medication to be taken on the morning and evening of days 4, 5, 6, and 7. Subjects returned to the clinic on day 7 and remained housed for approximately 24 hours after niacin dosing on day 8.

Dose Selection

This study investigated a pre-dosing regimen consisting of 243 mg of aspirin per day (81 mg q.a.m. and 162 mg q.p.m.) for four days and 20 mg/hr of aspirin started 6 hours before and continued for 5 hours after niacin dosing.

Preparation of Individual Outpatient Doses

Group A (Active period 1/Placebo period 2): 81 mg aspirin for the morning dose and 162 mg aspirin for the evening dose on days −4, −3, −2, −1; placebo for 81 mg aspirin for the morning dose and placebo for 162 mg aspirin for the evening dose on days 4, 5, 6 and 7.

Group B (Placebo period 1/Active period 2): placebo for 81 mg aspirin for the morning dose and placebo for 162 mg aspirin for the evening dose on days −4, −3, −2, −1; 81 mg aspirin for the morning dose and 162 mg aspirin for the evening dose on days 4, 5, 6 and 7.

Preparation of Inpatient Doses

Group A (Active period 1/Placebo period 2): twelve (12) 20 mg aspirin doses for day 1 and twelve (12) placebo for 20 mg aspirin for day 8;

Group B (Placebo period 1/Active period 2): twelve (12) placebo for 20 mg aspirin doses for day 1 and twelve (12) 20 mg aspirin for day 8.

Method of Administration

The study pharmacist dispensed blinded aspirin or placebo for oral administration during the outpatient portion of the study to the subject after the subject has completed all screening procedures. The medication was dispensed in individual envelopes for each dose, labeled with the subject number and the date and time to take the medication. Medications for days −4 to −1 were dispensed at the time of randomization. Medication for days 4 to 7 were dispensed prior to discharge on day 2.

Niacin, as well as blinded aspirin or placebo, for oral administration during the inpatient portion of the study (days 1 and 8) were dispensed by study pharmacist and administered to the subject by study personnel. Aspirin or placebo was administered hourly for twelve hours, starting 6 hours prior to the niacin dose.

Study duration was approximately twelve days. Prior to dosing, screening procedures were performed over a period of up to four weeks.

Packaging, Labeling and Storage

The study site sourced the aspirin and niacin products. The aspirin was over-encapsulated to obtain blinded supplies. “Matching” placebos were made by over-encapsulated artificial sweetener tablets of similar weight to the aspirin tablets. For some doses, the aspirin or artificial sweetener tablets were split into quarters or halves prior to over encapsulation to obtain smaller dosage amounts. Niacin was dosed in an open-label fashion.

Enrolled in the study were healthy volunteers (subjects in generally good health and free of any clinical disease that may interfere with study evaluations) of ages between 18 and 55.

Excluded from the study were subjects meeting the following criteria:

1. Subject had used aspirin within one month prior to screening or had used aspirin between screening and randomization;

2. Subject had used niacin or a niacin containing vitamin preparation with a dose of niacin greater than 50 mg within one month prior to screening or between screening and randomization;

3. Subject was pregnant or may become pregnant during the study;

4. Subject was perimenopausal or recently menopausal (last menstrual period within 12 months) with a history of hot flashes within last 12 months;

5. Subject had a history of sensitivity to aspirin, products containing aspirin, or other non-steroidal anti-inflammatory drugs;

6. Subject was currently using chronic medications or had used chronic medications within 30 days prior to screening;

7. Subject had a history of renal or hepatic disease;

8. Subject had participated in another investigational drug study within 30 days prior to current study;

9. Subject had a history of alcohol or drug abuse in past 2 years;

10. Subject had a positive blood alcohol or urine drug screen test;

11. Subject habitually smoked tobacco (>10 cigarettes per week).

Subject restrictions during the study were the following:

1. Subjects must have been willing to take study medication on an outpatient basis on days −4 through −1 and days 4 through 7. Subject must have been willing to remain housed in the clinic for 24 hours prior to dosing and for 24 hours following dose administration on days 1 and 8;

2. Subjects must have refrained from using aspirin (other than the aspirin used as study medication), ibuprofen or other NSAIDs, or acetaminophen from the screening visit through randomization and during the study;

3. Subjects were not allowed to drink hot beverages (e.g., coffee, tea, etc.) during the inpatient portion of the study;

4. Subjects must have refrained from alcohol consumption from screening through the end of the study (day 9);

5. Subjects must have refrained from smoking from screening through the end of the study (day 9).

Criteria for withdrawal from the study included but were not limited to the following:

1. Either at the investigator's request, for safety reasons, such as severe adverse reactions, or at the subject's request;

2. When the requirements of the protocol were not followed;

3. When a concomitant therapy liable to interfere with the results of the study was reported, or required, by the subject.

Concomitant Treatments

1. Chronic medications were not permitted;

2. The subject must have been washed out of prescription medications 7 days before the first dose;

3. No over the counter medications were permitted within 7 days of the first dose;

4. Aspirin (other than the aspirin used as study medication), ibuprofen or other NSAIDs and acetaminophen were expressly prohibited. Should a subject require analgesia for intercurrent headache or other symptom during the study, consideration was given for the use of a codeine preparation. Were a NSAID or acetaminophen specifically required, the subject was discontinued.

5. Vitamins and herbal supplements were not permitted during the study (from screening through the end of the study (day 9). The subjects have been washed out of vitamins 7 days before the first dose.

Assessment of Safety

Safety was assessed be assessing flushing episodes and by monitoring of other adverse events.

Assessment of Flushing

Episodes of flushing (including burning, itching, crawling sensation, pain and/or reddening of the skin, fever-like sensation) were recorded with start and end time and maximum severity captured. A 100 mm visual analog scale (0=no flushing symptoms; 100=very unpleasant symptoms) was used to capture severity as reported by the subject. For each subject, the number of episodes of flushing, maximum severity of all episodes of flushing, and total duration of flushing, from start of the first episode to the end of the last episode, was summarized by treatment regimen. These data were analyzed statistically for treatment effect using repeated measures techniques.

The obtained results have demonstrated that aspirin, in the regimen used, was effective in reducing flushing (53% reduction was achieved in mean flush intensity; and 77% reduction was achieved in median flush intensity).

### Example 2

Study

Provided is a randomized, double-blind, three-way cross-over study to investigate the anti-flushing effects of two aspirin regimens versus placebo on extended release niacin.

Product

The product is a niacin/aspirin oral dosage form.

Study Objective

To assess the ability of two aspirin regimens to decrease flushing associated with single-dose administration of extended-release niacin (Niaspan®).

Methodology

Extended-release niacin (2 g) was administered as a single oral dose with one of two aspirin regimens or placebo. Study subjects remained housed overnight at the clinical research center following niacin dosing to carefully monitor for flushing reactions and other adverse events. Each subject received all three dosing regimens in a randomized, three-way cross-over fashion.

Primary Endpoints

Treatment group comparisons of incidence, duration and severity of flushing following niacin dosing; and routine monitoring of adverse events.

Trial Design

Subjects have screening procedures performed up to 4 weeks prior to Period 1 Day (the first dosing visit), including assessment of flushing response to a 500 mg immediate release niacin (Niacor®) challenge. All qualified subjects received three single doses of extended-release niacin at least one week apart during three treatment periods. Subjects were randomized to receive one of the two aspirin regimens or placebo prior to their niacin dose on Day 1 of each treatment period. At randomization, subjects received blinded study medication to be taken in the evening of days −3 and −2. For each treatment period, subjects checked into the clinic on Day −1 and remained housed for at least 18 hours after niacin dosing on Day 1. Prior to leaving the clinic during treatment periods 1 and 2, subjects received blinded study medication to be taken in the morning and evening of Days −3 and −2 of the next treatment period. End of study procedures was performed on Day 2 of treatment period 3

A scheme of the study design and dosing regimens is shown below (Tables 1 and 2).

TABLE 1

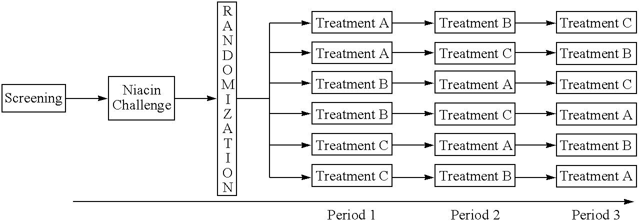

TABLE 2

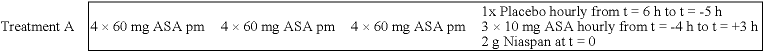

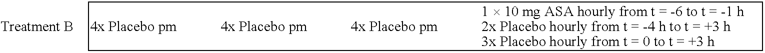

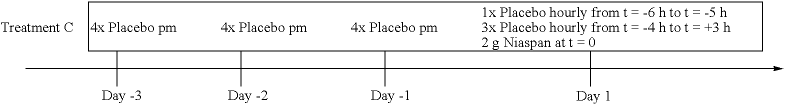

Dose Selection

Two different aspirin regimens and placebo were tested.

240 mg ASA q.p.m. for three days prior to niacin dosing plus 30 mg ASA per hour for eight hours, starting 4 hours prior to niacin dosing.

10 mg ASA per hour for 6 hours prior to niacin dosing. This regimen tested whether a lower dose of aspirin without an aspirin lead-in, and given entirely prior to niacin dosing, was sufficient to diminish flushing.

No aspirin. This regimen acted as the negative control.

Preparation of Individual Subject Doses

Treatment A

Number of Capsules/Tablets to Dispense per Dose

| | | | | Day 1 | | | | | | | | | |
|---|---|---|---|---|---|---|---|---|---|---|---|---|---|
| Medication | Day −3 | Day −2 | Day −1 | −6 h | −5 h | −4 h | −3 h | −2 h | −1 h | 0 | 1 h | 2 h | 3 h |
| 60 mg-ASA | 4 | 4 | 4 | 0 | 0 | 0 | 0 | 0 | 0 | 0 | 0 | 0 | 0 |
| Placebo for ASA | 0 | 0 | 0 | 1 | 1 | 0 | 0 | 0 | 0 | 0 | 0 | 0 | 0 |
| 10 mg ASA | 0 | 0 | 0 | 0 | 0 | 3 | 3 | 3 | 3 | 3 | 3 | 3 | 3 |
| Total # of Capsules | 4 | 4 | 4 | 1 | 1 | 3 | 3 | 3 | 3 | 3 | 3 | 3 | 3 |

Treatment B

Number of Capsules/Tablets to Dispense per Dose

| | | | | Day 1 | | | | | | | | | |
|---|---|---|---|---|---|---|---|---|---|---|---|---|---|
| Medication | Day −3 | Day −2 | Day −1 | −6 h | −5 h | −4 h | −3 h | −2 h | −1 h | 0 | 1 h | 2 h | 3 h |
| 60 mg-ASA | 0 | 0 | 0 | 0 | 0 | 0 | 0 | 0 | 0 | 0 | 0 | 0 | 0 |
| Placebo for ASA | 4 | 4 | 4 | 0 | 0 | 2 | 2 | 2 | 2 | 3 | 3 | 3 | 3 |
| 10 mg ASA | 0 | 0 | 0 | 1 | 1 | 1 | 1 | 1 | 1 | 0 | 0 | 0 | 0 |
| Total # of Capsules | 4 | 4 | 4 | 1 | 1 | 3 | 3 | 3 | 3 | 3 | 3 | 3 | 3 |

Treatment C

Number of Capsules/Tablets to Dispense per Dose

| | | | | Day 1 | | | | | | | | | |
|---|---|---|---|---|---|---|---|---|---|---|---|---|---|
| Medication | Day −3 | Day −2 | Day −1 | −6 h | −5 h | −4 h | −3 h | −2 h | −1 h | 0 | 1 h | 2 h | 3 h |
| 60 mg-ASA | 0 | 0 | 0 | 0 | 0 | 0 | 0 | 0 | 0 | 0 | 0 | 0 | 0 |
| Placebo for ASA | 4 | 4 | 4 | 1 | 1 | 3 | 3 | 3 | 3 | 3 | 3 | 3 | 3 |
| 10 mg ASA | 0 | 0 | 0 | 0 | 0 | 0 | 0 | 0 | 0 | 0 | 0 | 0 | 0 |
| Total # of Capsules | 4 | 4 | 4 | 1 | 1 | 3 | 3 | 3 | 3 | 3 | 3 | 3 | 3 |

Method of Administration

The study pharmacist dispensed blinded aspirin or placebo for oral administration during the outpatient portion of each study period for each subject after the subject has completed all screening procedures. The medication was dispensed in individual envelopes for each dose, labeled with the subject number and the date and time to take the medication. Medications for days −3 and −2 of treatment period 1 were dispensed at the time of randomization. Medication for Days −3 to −2 for treatment periods 2 and 3 were dispersed prior to discharge on Day 2 of the previous treatment period.

Niaspan®, as well as blinded aspirin or placebo, for oral administration during the inpatient portion of each treatment period (Day −1 and Day 1) were dispensed by study pharmacist and administered to the subject by study personnel. Aspirin or placebo were administered in the evening on Day −1 and hourly on Day 1 for 6 hours before, concurrently with, and hourly for 3 hours after the Niaspan® dose.

Study Duration

Study duration was approximately three weeks. Prior to dosing, screening procedures were performed over a period of up to four weeks.

Packaging, Labeling and Storage

The study site sourced the Niacor® (for the screening niacin challenge) and Niaspan® products which were dosed in an open-label fashion. Cerenis Therapeutics SA supplied aspirin capsules and a matching intermediate weight placebo.

Inclusion Criteria

Enrolled in the study were healthy volunteers (subjects in generally good health and free of any clinical disease that may interfere with study evaluations) of ages between 18 and 65 who exhibited at least minimal flushing following administration of 500 mg of immediate release niacin (defined as a VAS score ≧20 mm on a 100 mm scale) during the first three hours following niacin administration.

Exclusion Criteria

Excluded from the study were subjects meeting the following criteria:

1. Subject had used aspirin or other NSAID within 2 weeks prior to screening or had used aspirin between screening and randomization;

2. Subject had used niacin or a niacin containing vitamin preparation with a dose of niacin greater than 50 mg within one month prior to screening or between screening and randomization;

3. Subject was pregnant or may become pregnant during the study;

4. Subject was perimenopausal or recently menopausal (last menstrual period within 12 months) with a history of hot flashes within last 12 months;

5. Subject had a history of sensitivity to aspirin, products containing aspirin, or other non-steroidal anti-inflammatory drugs;

6. Subject was currently using chronic medications or had used chronic medications within 30 days prior to screening;

7. Subject had a history of renal or hepatic disease;

8. Subject had participated in another investigational drug study within 30 days prior to randomization;

9. Subject had a history of alcohol or drug abuse in past 2 years;

10. Subject had a positive blood alcohol or urine drug screen test at screening or upon admission to the clinic;

11. Subject habitually smoked tobacco (>10 cigarettes per week).

Restriction Criteria

Subject restrictions during the study were the following:

1. Subjects must have been willing to take study medication on an outpatient basis on Days −3 and −2 during each treatment period. Subject must have been willing to remain housed in the clinic for 24 hours prior to dosing and for at least 18 hours following dose administration during each treatment period;

2. Subjects must have refrained from using aspirin (other than the aspirin used as study medication), ibuprofen or other NSAIDs, or acetaminophen from the screening visit through randomization and during the study;

3. Subjects were not allowed to drink hot beverages (e.g., coffee, tea, etc.) during the inpatient portion of the study;

4. Subjects must have refrained from alcohol consumption from screening through the end of the study;

5. Subjects must have refrained from smoking from screening through the end of the study.

Withdrawal Criteria

Criteria for withdrawal from the study included but were not limited to the following:

1. Either at the investigator's request, for safety reasons, such as severe adverse reactions, or at the subject's request;

2. When the requirements of the protocol were not followed;

3. When a concomitant therapy liable to interfere with the results of the study was reported, or required, by the subject.

Concomitant Treatments

1. Chronic medications were not permitted;

2. The subject must have been washed out of prescription medications 7 days before the first niacin dose through the end of the study;

3. No over the counter medications were permitted within 7 days of the first niacin dose through the end of the study;

4. Aspirin (other than the aspirin used as study medication), ibuprofen or other NSAIDs and acetaminophen were expressly prohibited from screening to the end of the study. Should a subject require analgesia for intercurrent headache or other symptom during the study, consideration was given for the use of a codeine preparation. Were a NSAID or acetaminophen specifically required, the subject was discontinued.

5. Vitamins and herbal supplements were not permitted during the study (from screening through the end of the study. The subjects have been washed out of vitamins 7 days before the first niacin dose.

Assessment of Safety

Safety was assessed be assessing flushing episodes and by monitoring of other adverse events.

Assessment of Flushing

Episodes of flushing (including burning, itching, crawling sensation, pain and/or reddening of the skin, fever-like sensation) were recorded with start and end time maximum severity, and individual symptoms captured. A 100 mm visual analog scale (0=no flushing symptoms; 100=intolerable symptoms) was used to capture severity as reported by the subject. For each subject during each treatment period, the number of episodes of flushing, maximum severity of all episodes of flushing, and total duration of flushing, from start of the first episode to the end of the last episode, was calculated and summarized by treatment regimen. These data was analyzed statistically for treatment effect using repeated measures techniques.

Adverse Effects Reporting

An adverse event (AE) is any unfavorable and unintended sign including an abnormal laboratory finding, symptom, or disease temporally associated with the use of a medical product, whether or not considered related to the study drug. The severity rating was scaled based on the following categories:
mild—awareness of a symptom, but easily tolerated;
moderate—discomfort enough to cause interference with usual activity;
severe—incapacitating with inability to work or perform usual activity.

Results

A total of 54 subjects were enrolled in the study. Forty-six subjects completed all three dosing periods and have contributed data to the evaluation of flushing parameters. A total of 51 subjects were exposed to Treatment A (240 mg ASA), 50 to B (60 mg ASA), and 50 to C (Placebo); these subjects comprise the population for evaluation of safety parameters.

Flushing Response

Incidence of Flushing

Fewer subjects experienced a flushing response when they received 240 mg ASA (74%) compared to when they received 60 mg ASA (87%) or placebo (91%). The total number of flushing episodes that the 46 subjects experienced was also lower when they received 240 mg ASA (52 episodes) compared to 60 mg ASA (64 episodes) or placebo (68 episodes). The results are shown in FIGS. 8A and 8B.

Maximum Severity of Flushing

The maximum severity of flushing (measured on a 100 mm VAS) was lower when subjects received 240 mg ASA (mean 25.8 mm; median 20.0 mm) than when they received 60 mg ASA (mean 36.1 mm; median 35.0 mm) or placebo (mean 41.1 mm; median 35.5 mm). An analysis of variance (ANOVA) showed statistically significant differences between 240 mg ASA and placebo (p=0.0003) and between 240 mg ASA and 60 mg ASA (p=0.0130). The results are shown in FIGS. 9A and 9B.

Total Duration of Flushing

The total cumulative duration of flushing (from the start of the first episode until the end of the last episode) was shorter when subjects received 240 mg ASA (mean 67.1 min; median 44.5 min) than when they received 60 mg ASA (mean 101.8 min; median 84.0 min) or placebo (mean 127.6 min; median 87.5 min). ANOVA showed statistically significant differences between 240 mg ASA and placebo (p=0.0003) and between 240 mg ASA and 60 mg ASA (p=0.025). There was also a significant difference between 60 mg ASA and placebo (p=0.053). The results are shown in FIGS. 10A and 10B.

Number of Episodes of Flushing

Subjects experienced fewer episodes of flushing (mean 1.1) when they received 240 mg ASA compared to when they received 60 mg ASA (mean 1.4) or placebo (mean 1.5). ANOVA showed a statistically significant difference between 240 mg ASA and placebo (p=0.019). Furthermore, multinomial logistic regression showed that subjects were 2.1 times more likely to have fewer episodes of flushing on 240 mg ASA compared to placebo (p=0.01) and 1.7 times more likely to have fewer episodes with 240 mg ASA compared to 60 mg ASA (p=0.05). Subjects were 1.3 times more likely to have fewer episodes of flushing on 60 mg ASA compared to placebo (p>0.10).

Individual Flushing Symptoms

Subjects experienced a lower incidence of each flushing symptom when they received 240 mg ASA compared to when they received 60 mg ASA or placebo. Itching, crawling, reddening and fever-like sensation of the skin also occurred less frequently when subjects received 60 mg ASA compared to placebo. Results are shown in FIG. 11.

Safety Results

The overall incidence of treatment emergent adverse events (AEs) was similar for the three treatment regimens. All AEs were considered mild in intensity and no subject withdrew from the study due to an AE.

Summary of Adverse Events

| | 240 mg ASA | 60 mg ASA | Placebo |
|---|---|---|---|
| | (n = 51) | (n = 50) | (n = 50) |
| Number of AEs | 14 | 14 | 18 |
| Subjects with any AE | 12 (23.5%) | 10 (20.0%) | 13 (26.0%) |
| Subjects with any | 7 (13.7%) | 7 (14.0%) | 8 (16.0%) |
| CTM-related AE | | | |
| Subjects with Severe AEs | 0 (0.0%) | 0 (0.0%) | 0 (0.0%) |
| Subjects with Serious AEs | 0 (0.0%) | 0 (0.0%) | 0 (0.0%) |
| Subjects with AEs Leading | 0 (0.0%) | 0 (0.0%) | 0 (0.0%) |
| to Withdrawal | | | |

The most common adverse event was headache, which occurred in 4 subjects when receiving 240 mg ASA, 5 subjects when receiving 60 mg ASA and 3 subjects when receiving placebo. Adverse events involving gastrointestinal disorders occurred in 4 subjects while receiving 240 mg ASA, 3 while receiving 60 mg ASA, and 5 while receiving placebo.

### Example 3

Aspirin extended release and niacin modified release have been prepared by technologies described in U.S. Pat. No. 5,846,566, U.S. Pat. No. 5,603,957 and WO 03/030878 at Flamel Technologies (France).

Aspirin SR (ASA) is a extended release product presented as a white/white capsule for oral administration and containing 81 mg aspirin as aspirin microparticles. ASA is presented by three extended-release formulations as Aspirin SR capsule with 80% release within approximately 4-5 h (prototype A1), 6-7 h (prototype A2) and 9-10 h (prototype A3). A1, A2 and A3 prototypes have been manufactured with different coating ratios of the same coating composition. The coating composition was not pH sensitive, and the dissolution was not affected by the localization throughout the gastro-intestinal tract.

The manufacturing process of Aspirin SR was based on the coating of aspirin crystals of suitable shape and size supplied by Shandong as Refined acetylsalicylic acid grade 300/500. The quantitative composition of Aspirin SR 81 mg capsules of prototypes A1, A2 and A3 is given in tables 3-5.

TABLE 3
Composition of Aspirin SR (A1 prototype)

| | | | Centesimal |
|---|---|---|---|
| | | Composition | Composition |
| Ingredient | Function | (mg/capsule) | (%) |
| Refined | Active | 81.00 | 95.05 |
| acetylsalicylic acid | ingredient | | |
| grade 300/500 | | | |
| (aspirin) | | | |
| Ethylcellulose | Film coating | 2.18 | 2.56 |
| | agent | | |
| Castor oil | Plasticiser | 0.18 | 0.21 |
| Povidone | Film coating | 0.12 | 0.14 |
| | agent | | |
| Tartaric acid | Stabilising agent | 0.30 | 0.35 |
| Magnesium | Lubricant | 0.16 | 0.19 |
| stearate* | | | |
| Colloidal Silicon | Glidant | 0.43 | 0.50 |
| Dioxide | | | |
| Talc | Lubricant | 0.85 | 1.00 |
| Total | — | 85.22 | 100.00 |
| Hard gelatin* | — | — | 1 |
| capsule size 4 | | | |
| (white/white) | | | |

*vegetable origin

TABLE 4
Composition of Aspirin SR (A2 prototype)

| | | | Centesimal |
|---|---|---|---|
| | | Composition | Composition |
| Ingredient | Function | (mg/capsule) | (%) |
| Refined | Active | 81.00 | 93.98 |
| acetylsalicylic acid | ingredient | | |
| grade 300/500 | | | |
| (aspirin) | | | |
| Ethylcellulose | Film coating | 2.89 | 3.35 |
| | agent | | |
| Castor oil | Plasticiser | 0.24 | 0.28 |
| Povidone | Film coating | 0.16 | 0.19 |
| | agent | | |
| Tartaric acid | Stabilising agent | 0.40 | 0.46 |
| Magnesium | Lubricant | 0.21 | 0.24 |
| stearate* | | | |
| Colloidal Silicon | Glidant | 0.43 | 0.50 |
| Dioxide | | | |
| Talc | Lubricant | 0.86 | 1.00 |
| Total | — | 86.19 | 100.00 |
| Hard gelatin* | — | 1 | — |
| capsule size 4 | | | |
| (white/white) | | | |

*vegetable origin

TABLE 5
Composition of Aspirin SR (A3 prototype)

| | | | Centesimal |
|---|---|---|---|
| | | Composition | Composition |
| Ingredient | Function | (mg/capsule) | (%) |
| Refined | Active | 81.00 | 92.69 |
| acetylsalicylic acid | ingredient | | |
| grade 300/500 | | | |
| (aspirin) | | | |
| Ethylcellulose | Film coating | 3.76 | 4.30 |
| | agent | | |
| Castor oil | Plasticiser | 0.32 | 0.37 |
| Povidone | Film coating | 0.21 | 0.24 |
| | agent | | |
| Tartaric acid | Stabilising agent | 0.52 | 0.59 |
| Magnesium | Lubricant | 0.27 | 0.31 |
| stearate* | | | |
| Colloidal Silicon | Glidant | 0.44 | 0.50 |
| Dioxide | | | |
| Talc | Lubricant | 0.87 | 1.00 |
| Total | — | 87.39 | 100.00 |
| Hard gelatin* | — | 1 | — |
| capsule size 4 | | | |
| (white/white) | | | |

*vegetable origin

Aspirin prototypes were prepared identically, using a different excipient ratio. The manufacturing process involved the three steps: coating of the aspirin crystals, encapsulation of Aspirin SR microparticles, and packaging.

Coating: The aspirin crystals were coated using a spray-coating technique in a bottom-spray fluidized bed equipment. The coating suspension was prepared by mixing the coating excipients in an acetone/isopropyl alcohol mixture in a stainless steel vessel equipped with a stirring device. The suspension was sprayed at room temperature onto the aspirin crystals, in a fluidized bed apparatus working under nitrogen. During the process, the solvents were evaporated by the fluidization stream, allowing the composition to deposit around the crystals as a continuous coating membrane, thus forming the Aspirin SR microparticles.

Encapsulation: The Aspirin SR microparticles were mixed with the capsule filling excipients in order to obtain a free flowing blend. This blend was achieved in a drum-type blender of appropriate capacity. The resulting blend was filled into hard gelatin capsules, using a semi-automatic rotating machine. Each capsule contained 81 mg of aspirin.

Table 6 shows the specification at release for Aspirin SR 81 mg capsule type A1, A2 and A3.

TABLE 6
Specification at release - Aspirin SR 81 mg capsules A1, A2 and A3

| | | Specification | | |
|---|---|---|---|---|
| | | Formulation | Formulation | Formulation |
| Test | Method | A1 | A2 | A3 |
| Appearance | Internal | White capsules containing white to | | |
| | method | slightly yellow microparticles | | |
| Identification by HPLC | Internal | Retention time similar to that of the | | |
| | method | reference solution | | |
| Dissolution test at pH 6.8 (% | Internal | Similar to | Similar to | Similar to |
| acetylsalicylic acid) | method | the | the | the |
| | | reference | reference | reference |
| | | profile A1 | profile A2 | profile A3 |

| Uniformity of dosage units | | USP | Complies with USP/NF current edition |
|---|---|---|---|
| (weight variation) | | <905> | <905> |
| Related | Salicylic acid | Internal | ≦0.5 |
| substances by | (SA) (%) | method | |
| HPLC | Acetylsalicyl | | ≦0.1 |
| (Mass ratio/ | salicylic acid | | |
| Acetylsalicylic | (ASSA) (%) | | |
| acid) | Salicylsalicylic | | ≦0.1 |
| | acid (SSA) | | |
| | (%) | | |
| | Total | | ≦0.7 |
| | impurities | | |
| | (%) | | |
| Assay (by HPLC) | | Internal | 72.9 to 89.1 |
| (mg/capsule) | | method | |
| Acetone and | Acetone | Internal | <20 000 |
| Isopropanol | (ppm) | method | |
| residual by GC | Isopropanol | | <20 000 |
| | (ppm) | | |
| | Sum of | | <20 000 |
| | residual | | |
| | solvents | | |
| | (ppm) | | |
| Microbial | Total aerobic | Internal | ≦1000 |
| contamination | microbial | method | |
| | count | Complies | |
| | (CFU/g) | with | |
| | Total | USP | ≦100 |
| | combined | <61> | |
| | yeasts and | and | |
| | molds count | <62> | |
| | (CFU/g) | | |
| | Escherichia | | Absence |
| | coli | | |

Three target dissolution profiles have been calculated for each type of Aspirin SR 81 mg capsules and are presented in Table 7 and FIG. 2.

TABLE 7
Target dissolution profiles for Aspirin SR 81 mg capsule

| | Aspirin SR 81 mg | | |
|---|---|---|---|
| Dissolution | capsule type | | |
| time (hour) | A1 | A2 | A3 |
| 0 | 0.0 | 0.0 | 0.0 |
| 0.5 | 21.2 | 15.9 | 11.7 |
| 1 | 35.3 | 27.1 | 20.3 |
| 1.5 | 46.2 | 36.2 | 27.6 |
| 2 | 54.9 | 43.9 | 33.9 |
| 2.5 | 62.0 | 50.4 | 39.6 |
| 3 | 67.8 | 56.0 | 44.6 |
| 3.5 | 72.6 | 60.9 | 49.1 |
| 4 | 76.7 | 65.2 | 53.1 |
| 4.5 | 80.0 | 68.9 | 56.8 |
| 5 | 82.9 | 72.2 | 60.2 |
| 5.5 | 85.3 | 75.1 | 63.2 |
| 6 | 87.4 | 77.7 | 66.0 |
| 7 | 90.6 | 82.0 | 70.9 |
| 8 | 93.0 | 85.5 | 75.0 |
| 9 | 94.7 | 88.2 | 78.5 |
| 10 | 96.0 | 90.4 | 81.4 |
| 11 | 97.0 | 92.1 | 83.9 |
| 12 | 97.7 | 93.6 | 86.1 |
| 13 | 98.3 | 94.7 | 87.9 |
| 14 | 98.7 | 95.7 | 89.5 |
| 15 | 99.0 | 96.4 | 90.9 |
| 16 | 99.2 | 97.1 | 92.1 |

Niacin MR capsule is a modified release product presented as a white/white capsule for oral administration and containing 500 mg niacin microparticles.

Formulation development for Niacin MR produced a series of modified-release niacin batches with different lag times between swallowing and release starting point, which allowed selection of the targeted release sites. The products are obtained by coating niacin particles with a composition suitable for safe passage through the stomach after swallowing, then allowing release in different gastrointestinal tract (GIT) segments. The product behavior (resistance in the stomach combined with release in a further specific location of GIT) is based on an association of three components in the coating composition: two hydrophilic methacrylic polymers, with different pH dependent solubilities, and one hydrophobic material. It was inferred that the difference in lag times in vivo between the three formulae was determined by the different polymer ratios in the coating composition.

The manufacturing process is based on the preparation of niacin MR 500 mg capsule prototypes N1, N2 and N3 by coating niacin granular of a suitable shape and size supplied by Lonza as Niacin USP granular special.

The quantitative compositions of Niacin MR 500 mg capsules for formulation prototypes N1, N2 and N3 are presented in tables 8-10 (below).

TABLE 8
Composition of Niacin MR 500 mg capsules N1

| | | | Centesimal |
|---|---|---|---|
| | | Composition | Composition |
| Ingredient | Function | (mg/capsule) | (%) |
| Niacin USP | Active ingredient | 500.00 | 81.68 |
| granular special | | | |
| (nicotinic acid) | | | |
| Methacrylic Acid | Film coating | 53.03 | 8.66 |
| Copolymer type C | agent | | |
| (Eudragit ® L100- | | | |
| 55) | | | |
| Methacrylic Acid | Film coating | 10.61 | 1.73 |
| Copolymer type B | agent | | |
| (Eudragit ® S100) | | | |
| Hydrogenated | Film coating | 42.42 | 6.93 |
| Cottonseed Oil | agent | | |
| (Lubritab ®) | | | |
| Magnesium | Lubricant | 3.06 | 0.50 |
| stearate* | | | |
| Colloidal Silicon | Lubricant | 3.06 | 0.50 |
| Dioxide | | | |

*vegetable origin

TABLE 9
Composition of Niacin MR 500 mg capsules N2

| | | | Centesimal |
|---|---|---|---|
| | | Composition | Composition |
| Ingredient | Function | (mg/capsule) | (%) |
| Niacin USP | Active | 500.00 | 79.20 |
| granular special | ingredient | | |
| (nicotinic acid) | | | |
| Methacrylic Acid | Film coating | 50.00 | 7.92 |
| Copolymer type C | agent | | |
| (Eudragit ® L100- | | | |
| 55) | | | |
| Methacrylic Acid | Film coating | 25.00 | 3.96 |
| Copolymer type B | agent | | |
| (Eudragit ® S100) | | | |
| Hydrogenated | Film coating | 50.00 | 7.92 |
| Cottonseed Oil | agent | | |
| (Lubritab ®) | | | |
| Magnesium | Lubricant | 3.16 | 0.50 |
| stearate* | | | |
| Colloidal Silicon | Lubricant | 3.16 | 0.50 |
| Dioxide | | | |

*vegetable origin

TABLE 10
Composition of Niacin MR 500 mg capsules N3

| | | | Centesimal |
|---|---|---|---|
| | | Composition | Composition |
| Ingredient | Function | (mg/capsule) | (%) |
| Niacin USP | Active ingredient | 500.00 | 79.20 |
| granular special | | | |
| (nicotinic acid) | | | |
| Methacrylic Acid | Film coating | 25.00 | 3.96 |
| Copolymer type C | agent | | |
| (Eudragit ® L100- | | | |
| 55) | | | |
| Methacrylic Acid | Film coating | 50.00 | 7.92 |
| Copolymer type B | agent | | |
| (Eudragit ® S100) | | | |
| Hydrogenated | Film coating | 50.00 | 7.92 |
| Cottonseed Oil | agent | | |
| (Lubritab ®) | | | |
| Magnesium | Lubricant | 3.16 | 0.50 |
| stearate* | | | |
| Colloidal Silicon | Lubricant | 3.16 | 0.50 |
| Dioxide | | | |

*vegetable origin

The niacin prototypes were prepared identically, using a different excipient ratio. The manufacturing process involved the three steps: coating of Niacin USP granular special; encapsulation of Niacin MR microparticles; and packaging.

Coating: the niacin granules were coated using a spray-coating technique in a bottom-spray fluidized bed equipment.

The coating solution is prepared by dissolving the coating excipients in hot isopropyl alcohol using a jacketed appropriate vessel equipped with a stirring device. The solution is sprayed at about 75° C. onto the niacin granules, in the fluidized bed apparatus. During the process, the solvent is evaporated by the fluidization air stream, allowing the composition to deposit around the granules as a continuous coating membrane, thus forming the Niacin MR microparticles.

Encapsulation: the Niacin MR microparticles were mixed with the capsule filling excipients in a drum-type blender of appropriate capacity. The resulting blend is filled into hard gelatin capsules, using a semi-automatic machine. Each capsule contained 500 mg of niacin microparticles.

Table 11 below shows the specification at release for Niacin MR 500 mg capsules.

TABLE 11
Specification at release of Niacin MR 500 mg capsules N1, N2 and N3

| | | Specification | | |
|---|---|---|---|---|
| | | Formulation | Formulation | Formulation |
| Test | Method | N1 | N2 | N3 |

| Appearance | | Internal | White capsules containing a white to slightly |
|---|---|---|---|
| | | method | yellow microparticles |
| Identification by HPLC | | Internal | Retention time similar to that of the |
| | | method | reference solution |
| Dissolution test at 0.1 NHCl | | Internal | ≦30% |
| (% nicotinic acid) - | | method | |
| 5 hours | | | |
| Dissolution test at pH 7.4 | | Internal | ≧80% |
| (% nicotinic acid) - | | method | |
| 5 hours | | | |
| Uniformity of dosage units | | USP | Complies with USP/NF current edition |
| (weight variation) | | <905> | <905> |
| Related | Individual | Internal | Each individual degradation product will be |
| substances by | degradation | method | reported from the reporting level of 0.10% |
| HPLC | products | | and identified from the identification level of |
| | (%) | | 0.2%. |
| | Total | | ≦5% |
| | degradation | | |
| | products | | |
| | (%) | | |
| Assay (by HPLC) | | Internal | 450 to 550 |
| (mg/capsule) | | method | |
| Isopropyl alcohol residual | | Internal | <19 000 |
| by GC (ppm) | | method | |
| Microbial | Total | Internal | ≦1 000 |
| contamination | aerobic | method | |
| | microbial | Complies | |
| | count | with USP | |
| | (CFU/g) | <61> and | |
| | Total | <62> | ≦100 |
| | combined | | |
| | yeasts | | |
| | and | | |
| | molds | | |
| | count | | |
| | (CFU/g) | | |
| | Escherichia | | Absence |
| | coli | | |

Combination formulations are obtained by mixing active principle microparticles with appropriate excipients necessary for the appropriate formulation. For example, capsules are prepared by mixing one aspirin prototype and one niacin prototype directly into the capsule to give a non homogenous mixture of microparticles.

### Example 4

Provided are ASA and niacin plasma pharmacokinetic profiles.

Provided below are the ASA plasma pharmacokinetic profiles. Cₘₐₓ at different release rates for various doses are about:
160 mg: fast—290 ng/ml, intermediate—190 ng/ml, slow—130 ng/ml;
240 mg: fast—420 ng/ml; intermediate—290 ng/ml: slow—200 ng/ml;
324 mg: fast—570 ng/ml; intermediate—390 ng/ml; slow—280 ng/ml.

Provided below are aspirin AUC plasma pharmacokinetic profiles. AUC at various doses are about:
160 mg: AUC=800 ngml⁻¹h;
240 mg: AUC=1200 ngml⁻¹h;
324 mg: AUC=1600 ngml⁻¹h.

Provided below are salicylic acid plasma pharmacokinetic profiles. Cₘₐₓ at different release rates for various doses are about:
160 mg: fast—2600 ng/ml, intermediate—1700 ng/ml, slow—1200 ng/ml;
240 mg: fast—3700 ng/ml; intermediate—2600 ng/ml: slow—1800 ng/ml;
324 mg: fast—5000 ng/ml; intermediate—3500 ng/ml; slow—2400 ng/ml.

Provided below are aspirin AUC plasma pharmacokinetic profiles. AUC at various doses are about:
160 mg: AUC=11000 ngml⁻¹h;
240 mg: AUC=16300 ngml⁻¹h;
324 mg: AUC=22000 ngml⁻¹h.

Provided below is a niacin plasma pharmacokinetic profile (the dose of niacin is 2000 mg). In one embodiment, Cₘₐₓ is in the range of 3-13 μg/ml. In another embodiment, Cₘₐₓ is in the range of 6-9 μg/ml.

Provided below are Tₘₐₓ meanings for a niacin dose of 2000 mg. In one embodiment, Tₘₐₓ is about 8 hours. In another embodiment, Tₘₐₓ is about 9 hours. In yet another embodiment, Tₘₐₓ is about 10.5 hours.

Provided below is a niacin AUC profile (the dose of niacin is 2000 mg). In one embodiment, AUC is in the range of 8-52 μg/ml⁻¹h. In another embodiment, AUC is in the range of 15-35 μg/ml⁻¹h. In yet another embodiment, AUC is in the range of 20-25 μg/ml⁻¹h.

Provided below is a plasma pharmacokinetic profile for nicotinuric acid (the dose is 2000 mg). In one embodiment, Cₘₐₓ is in the range of 1.25-5.2 μg/ml. In another embodiment, Cₘₐₓ is in the range of 2.5-3.5 μg/ml.

Provided below are Tₘₐₓ for nicotinuric acid following a 2000 mg dose of niacin. In one embodiment, Tₘₐₓ is about 8 hours. In another embodiment, Tₘₐₓ is about 9 hours. In yet another embodiment, Tₘₐₓ is about 10.5 hours.

Provided below is a nicotinuric acid AUC profile (the dose of niacin is 2000 mg). In one embodiment, AUC is in the range of 6-27 μg/ml⁻¹h. In another embodiment, AUC is in the range of 12-18 μg/ml⁻¹h. In yet another embodiment, AUC is in the range of 14-16 μg/ml⁻¹h.

Based upon the foregoing disclosure, it should now be apparent that the use of the compositions and methods described herein will carry out the objects set forth hereinabove. It is, therefore, to be understood that any variations the compositions and methods fall within the scope of the provided methods and compositions, and thus their scope will include all modifications and variations that may fall within the scope of the attached claims.

What is claimed:

1. A pharmaceutical composition, comprising niacin and aspirin, wherein the total daily dose of aspirin is about 80 mg to about 500 mg, wherein the aspirin is released from the composition over about 2 to about 16 hours, and a pharmaceutically acceptable carrier.

2. The pharmaceutical composition of claim 1, wherein the total daily dose of aspirin is about 80 mg to about 320 mg.

3. The pharmaceutical composition of claim 2, wherein the total daily dose of aspirin is about 100 mg to about 140 mg.

4. The pharmaceutical composition of claim 3, wherein the total daily dose of aspirin is about 120 mg.

5. The pharmaceutical composition of claim 1, wherein the total daily dose of aspirin is about 120 mg to about 240 mg.

6. The pharmaceutical composition of claim 5, wherein the total daily dose of aspirin is about 140 mg to about 200 mg.

7. The pharmaceutical composition of claim 6, wherein the total daily dose of aspirin is about 160 mg to about 162 mg.

8. The pharmaceutical composition of claim 1, wherein the total daily dose of aspirin is about 180 mg to about 300 mg.

9. The pharmaceutical composition of claim 8, wherein the total daily dose of aspirin is about 200 mg to about 260 mg.

10. The pharmaceutical composition of claim 9, wherein the total daily dose of aspirin is about 240 mg to about 243 mg.

11. The pharmaceutical composition of claim 9, wherein the total daily dose of aspirin is about 240 mg.

12. The pharmaceutical composition of claim 1, wherein the total daily dose of aspirin is about 200 mg to about 360 mg.

13. The pharmaceutical composition of claim 12, wherein the total daily dose of aspirin is about 300 mg to about 340 mg.

14. The pharmaceutical composition of claim 13, wherein the total daily dose of aspirin is about 320 mg to about 324 mg.

15. The pharmaceutical composition of claim 1, wherein the total daily dose of aspirin is released over a period of up to 16 hours.

16. The pharmaceutical composition of claim 1, wherein the total daily dose of aspirin is about 120 mg that is released over a period of about 2 to about 6 hours.

17. The pharmaceutical composition of claim 1, wherein the total daily dose of aspirin is about 120 mg that is released over a period of up to 6 hours.

18. The pharmaceutical composition of claim 1, wherein the total daily dose of aspirin is about 120 mg that is released over a period of up to 4 hours.

19. The pharmaceutical composition of claim 1, wherein the total daily dose of aspirin is about 120 mg that is released over a period of up to 3 hours.

20. The pharmaceutical composition of claim 1, wherein the total daily dose of aspirin is about 120 mg that is released over a period of up to 2 hours.

21. The pharmaceutical composition of claim 1, wherein the total daily dose of aspirin is about 160 mg that is released over a period of about 2 to about 8 hours.

22. The pharmaceutical composition of claim 1, wherein the total daily dose of aspirin is about 160 mg that is released over a period of up to 8 hours.

23. The pharmaceutical composition of claim 1, wherein the total daily dose of aspirin is about 160 mg that is released over a period of up to 5-6 hours.

24. The pharmaceutical composition of claim 1, wherein the total daily dose of aspirin is about 160 mg that is released over a period of up to 4 hours.

25. The pharmaceutical composition of claim 1, wherein the total daily dose of aspirin is about 160 mg that is released over a period of up to 2-3 hours.

26. The pharmaceutical composition of claim 1, wherein the total daily dose of aspirin is about 240 mg that is released over a period of about 4 to about 12 hours.

27. The pharmaceutical composition of claim 1, wherein the total daily dose of aspirin is about 240 mg that is released over a period of up to 12 hours.

28. The pharmaceutical composition of claim 1, wherein the total daily dose of aspirin is about 240 mg that is released over a period of up to 8 hours.

29. The pharmaceutical composition of claim 1, wherein the total daily dose of aspirin is about 240 mg that is released over a period of up to 6 hours.

30. The pharmaceutical composition of claim 1, wherein the total daily dose of aspirin is about 240 mg that is released over a period of up to 4 hours.

31. The pharmaceutical composition of claim 1, wherein the total daily dose of aspirin is about 320 mg that is released over a period of about 4 to about 16 hours.

32. The pharmaceutical composition of claim 1, wherein the total daily dose of aspirin is about 320 mg that is released over a period of up to 16 hours.

33. The pharmaceutical composition of claim 1, wherein the total daily dose of aspirin is about 320 mg that is released over a period of up to 10-11 hours.

34. The pharmaceutical composition of claim 1, wherein the total daily dose of aspirin is about 320 mg that is released over a period of up to 8 hours.

35. The pharmaceutical composition of claim 1, wherein the total daily dose of aspirin is about 320 mg that is released over a period of up to 5-6 hours.

36. The pharmaceutical composition of claim 1, wherein the total daily dose of aspirin is about 320 mg that is released over a period of up to 4 hours.

37. A pharmaceutical composition comprising niacin and aspirin, further comprising a lipid-lowering drug other than niacin, wherein the total daily dose of aspirin is about 80 mg to about 400 mg, wherein the aspirin is released from the composition over about 2 to about 16 hours, and a pharmaceutically acceptable carrier.

38. The pharmaceutical composition of claim 37, wherein the total daily dose of aspirin is released over a period of up to 12 hours.

39. The pharmaceutical composition of claim 37, wherein the lipid-lowering drug is a statin, fibrate, bile acid sequestrant or cholesterol absorption inhibitor.

40. The pharmaceutical composition of claim 39, wherein the lipid-lowering drug is a statin.

41. The pharmaceutical composition of claim 40, wherein the statin is atorvastatin.

42. A pharmaceutical composition comprising aspirin microparticles and niacin microparticles, wherein the aspirin microparticles have a first release profile and the niacin microparticles have a second release profile.

43. The pharmaceutical composition of claim 42, wherein the first release profile is based on release of aspirin in a pH independent fashion.

44. The pharmaceutical composition of claim 42, wherein about 80% of aspirin is released over a 4-10 hour period.

45. The pharmaceutical composition of claim 44, wherein about 80% of aspirin is released over a 4-8 hour period.

46. The pharmaceutical composition of claim 44, wherein about 80% of aspirin is released over a 4-5 hour period.

47. The pharmaceutical composition of claim 43, wherein about 80% of aspirin is released over a 6-7 hour period.

48. The pharmaceutical composition of claim 44, wherein about 80% of aspirin is released over a 9-10 hour period.

49. The pharmaceutical composition of claim 42, wherein the second release profile is based on release of niacin in a pH dependent fashion.

50. The pharmaceutical composition of claim 49, wherein the pH for release of the niacin is in the range of about 5.5 and about 8.0.

51. The pharmaceutical composition of claim 50, wherein the pH for release of the niacin is about 6.0.

52. The pharmaceutical composition of claim 50, wherein the pH for release of the niacin is about 6.5.

53. The pharmaceutical composition of claim 50, wherein the pH for release of the niacin is about 7.0.

54. The pharmaceutical composition of claim 50, wherein the pH for release of the niacin is about 7.5.

55. A pharmaceutical composition comprising a mixture of aspirin microparticles and niacin microparticles designed to keep aspirin and niacin physically separated, wherein the aspirin microparticles and the niacin microparticles are administered at the same time as one tablet or capsule.

56. The pharmaceutical composition of claim 55, wherein the tablet or capsule is given after 6 pm.

57. The pharmaceutical composition of claim 55, wherein the tablet or capsule is given after 12 am.

58. A pharmaceutical composition comprising niacin microparticles having a pH-dependent release profile, and aspirin microparticles having a pH-independent release profile, wherein the niacin microparticles have a reduced capacity to provoke a flushing reaction in a subject, wherein the aspirin is present in an amount effective to reduce a cutaneous flushing caused by the niacin, and wherein there is a lag time between release of aspirin and niacin following administration of the composition.

59. The pharmaceutical composition of claim 58, wherein the total daily dose of aspirin is about 80 mg to about 500 mg that is released based on an aspirin release profile, wherein about 70% to 90% of aspirin AUC is released over a period of time of about 2 to about 16 hours following administration of the composition.

60. The pharmaceutical composition of claim 58, wherein the total daily dose of aspirin is released from the composition based on an aspirin release profile, wherein the aspirin concentration in plasma is greater than 5% of Cmax over a period of time of about 2 to about 16 hours following administration of the composition.

61. The pharmaceutical composition of claim 58, wherein the total daily dose of aspirin is released from the composition based on an aspirin release profile, wherein the aspirin concentration in plasma is greater than 10% of Cmax over a period of time of about 2 to about 16 hours following administration of the composition.

62. The pharmaceutical composition of claim 59, wherein the total daily dose of aspirin is about 80 mg to about 400 mg.

63. The pharmaceutical composition of claim 62, wherein the total daily dose of aspirin is about 80 mg to about 325 mg.

64. The pharmaceutical composition of claim 63, wherein the total daily dose of aspirin is about 324 mg.

65. The pharmaceutical composition of claim 62, wherein the total daily dose of aspirin is about 80 mg to about 260 mg.

66. The pharmaceutical composition of claim 65, wherein the total daily dose of aspirin is about 243 mg.

67. The pharmaceutical composition of claim 62, wherein the total daily dose of aspirin is about 80 mg to about 200 mg.

68. The pharmaceutical composition of claim 67, wherein the total daily dose of aspirin is about 162 mg.

69. The pharmaceutical composition of claim 62, wherein the total daily dose of aspirin is about 80 mg to about 100 mg.

70. The pharmaceutical composition of claim 69, wherein the total daily dose of aspirin is about 81 mg.

71. The pharmaceutical composition of claim 59, wherein the period of time is about 3 to about 12 hours.

72. The pharmaceutical composition of claim 71, wherein the period of time is about 9 to about 10 hours.

73. The pharmaceutical composition of any one of claims 59, wherein the period of time is about 4 to about 8 hours.

74. The pharmaceutical composition of claim 71, wherein the period of time is about 6 to about 7 hours.

75. The pharmaceutical composition of claim 71, wherein the period of time is about 4 to about 5 hours.

76. A pharmaceutical composition comprising aspirin and niacin that decreases a niacin treatment drop-out rate, wherein the composition is as defined in claim 1.

77. A pharmaceutical composition comprising aspirin and niacin that allows a patient to tolerate a higher dose of aspirin, wherein the composition is as defined in claim 1.

78. A method for preventing or treating niacin-induced flushing in a subject, comprising administering to the subject a flush-inducing amount of niacin and a flush-reducing amount of aspirin, wherein the total daily dose of aspirin is about 80 mg to about 500 mg.

79. The method of claim 78, wherein the total daily dose of aspirin is released from the composition based on an aspirin release profile, wherein 70 to 90% of aspirin AUC is released over a period of time of about 2 to about 16 hours following administration of the aspirin.

80. The method of claim 78, wherein the total daily dose of aspirin is released from the composition based on an aspirin release profile, wherein the aspirin concentration in plasma is greater than 5% of Cmax over a period of time of about 2 to about 16 hours following administration of the aspirin.

81. The method of claim 78, wherein the total daily dose of aspirin is released from the composition based on an aspirin release profile, wherein the aspirin concentration in plasma is greater than 10% of Cmax over a period of time of about 2 to about 16 hours following administration of the aspirin.

82. The method of claim 79, wherein the period of time is about 3 to about 12 hours.

83. The method of claim 82, wherein the period of time is about 9 to about 10 hours.

84. The method of claim 79, wherein the period of time is about 4 to about 8 hours.

85. The method of claim 84, wherein the period of time is about 6 to about 7 hours.

86. The method of claim 85, wherein the period of time is about 4 to about 5 hours.

87. The method of claim 78, wherein the subject is predosed on the day of niacin therapy with an aspirin regimen, wherein about 80% of niacin AUC is not released until after about 16 hours of predosing with aspirin.

88. The method of claim 78, wherein the subject is predosed on the day of niacin therapy with an aspirin regimen, wherein about 90% of niacin AUC is not released until after about 16 hours of predosing with aspirin.

89. The method of claim 78, wherein the plasma concentration of niacin is less than 20% of Cmax until after about 16 hours of predosing with aspirin.

90. The method of claim 78, wherein the plasma concentration of niacin is less than 10% of Cmax until after about 16 hours of predosing with aspirin.

91. The method of claim 87, wherein a period of time is about 12 hours.

92. The method of claim 87, wherein a period of time is about 10 hours.

93. The method of claim 87, wherein a period of time is about 8 hours.

94. The method of claim 87 to 90, wherein a period of time is about 6 hours.

95. The method of claim 87, wherein a period of time is about 5 hours.

96. The method of claim 87, wherein a period of time is about 4 hours.

97. The method of claim 87, wherein a period of time is about 3 hours.

98. The method of claim 87, wherein a period of time is about 2 hours.

99. The method of claim 87, wherein a period of time is about 1 hour.

100. The method of claim 78, further comprising a lipid-lowering drug other than niacin.

101. The method of claim 100, wherein the lipid-lowering drug is a statin, fibrate, bile acid sequestrant or cholesterol absorption inhibitor.

102. The method of claim 101, wherein the lipid-lowering drug is a statin.

103. The method of claim 102, wherein the statin is atorvastatin.

104. A method for preventing or treating niacin-induced flushing in a subject, comprising administering to the subject a flush-inducing amount of niacin and a flush-reducing amount of aspirin, wherein the total daily dose of aspirin is about 80 mg to about 500 mg, and wherein the aspirin is continuously administered before, during and after niacin administration.

105. The method of claim 104, wherein the aspirin is continuously administered before and during niacin administration.

106. A method for reducing at least one flushing symptom related to niacin therapy in a subject comprising administering to said subject a niacin/aspirin formulation of claim 1, wherein the flushing symptom is burning, itching, tingling, crawling, reddening or fever-like symptoms.

107. A method for decreasing prostaglandin related side effects in a subject, comprising administering to said subject a niacin/aspirin formulation of claim 1.

108. A method for decreasing a discontinuation rate of niacin treatment by a subject, comprising administering to said subject a niacin/aspirin formulation of claim 1.

109. A method for increasing patient compliance with niacin treatment, comprising administering to said patient a niacin/aspirin formulation of claim 1.

110. A method for treating atherosclerosis in a patient, comprising administering to said patient a niacin/aspirin formulation of claim 1.

111. A method for treating a disease related to a low HDL profile in a patient, comprising administering to said patient a niacin/aspirin formulation of claim 1.

112. A modified release nicotinic acid formulation with a lag phase before niacin delivery suitable for oral administration once a day dosing for treating hyperlipidemia without causing drug-induced hepatotoxicity to a level which would require said nicotinic acid formulation to be discontinued, said modified release nicotinic acid formulation exhibiting a release pattern characterized by two phases when a convoluted plasma curve for nicotinic acid released from the said modified release nicotinic acid formulation is deconvoluted using the Wagner-Nelson method, a lag phase and an extended release phase,
wherein the lag phase is characterized in that less than 10% of the nicotinic acid dose administered is absorbed between about 2 and about 4 hours following ingestion;
wherein the extended release phase being characterized in that more than about 20% but less than 78% of the nicotinic acid administered being absorbed between about 7 and 8 hours following ingestion; and
wherein less than 90% of the nicotinic acid administered being absorbed by 9 hours following ingestion.

113. The modified release nicotinic acid formulation of claim 112, wherein the lag phase is characterized by plasma levels being below 20% of the C_{MAX} for at least 3 hours after the time of ingestion and up to 16 hours following ingestion;
wherein the extended release phase being characterized by plasma levels following the lag phase being maintained above 20% of the C_{MAX} for a period of at least 3 hours but less than 8 hours; and
wherein plasma levels following the extended release phase being less than 5% of the C_{MAX} by hour 24.

114. The modified release nicotinic acid formulation of claim 112, wherein said modified release nicotinic acid formulation exhibiting a release pattern,
wherein the nicotinic acid absorption mean is between 1% and 10% of the nicotinic acid dose administered during the lag phase of between ingestion and 3 and to 8 hours following ingestion; and
wherein less than 90% of the nicotinic acid dose administered is absorbed at about 7.5 hours following ingestion.

115. The modified release nicotinic acid formulation of claim 112, wherein said modified release nicotinic acid formulation exhibiting a release pattern,
wherein the lag phase being characterized by plasma levels below 20% of the C_{MAX} for at least 3 hours after the time of ingestion and up to 16 hours following ingestion; and
wherein the extended release phase being characterized by a T_{MAX} of at least 6 hours but less than 20 hours following ingestion.

116. A method of treating or preventing a disease or disorder selected from the group consisting of:
a) disorders of lipoprotein metabolism, wherein the disorder is dyslipidemia, dyslipoproteinemia, lipoprotein overproduction or deficiency, elevation of total cholesterol, elevation of low density lipoprotein concentration, elevation of triglyceride concentration, lipid elimination in bile, metabolic disorder, phospholipid elimination in bile, oxysterol elimination in bile, abnormal bile production, or peroxisome proliferator activated receptor-associated disorder;
(b) disorders of glucose metabolism, wherein the disorder is insulin resistance, impaired glucose tolerance, impaired fasting glucose levels in blood, diabetes mellitus, lipodystrophy, central obesity, peripheral lipoatrophy, diabetic nephropathy, diabetic retinopathy, renal disease, or septicemia;
(c) cardiovascular disorders and related vascular disorders, wherein the disorder is atherosclerosis, hypertension, coronary artery disease, myocardial infarction, arrhythmia, atrial fibrillation, heart valve disease, heart failure, cardiomyopathy, myopathy, pericarditis, impotence, or thrombotic disorder;
(d) modulating inflammation markers and/or C-reactive protein and related disorders, wherein the disorder is inflammation, ischemic necrosis, colon cancer, or thrombotic disorder; and
(e) aging, Alzheimer's Disease, Parkinson's disease, pancreatitis, and pancreatitius;
comprising administering the pharmaceutical composition of claim 1.

117. An aspirin microcapsule having a coating ratio of about 2.5% to about 15%, wherein the amount of acetylsalicylic acid is about 80% to about 98%, the amount of ethylcellulose is about 1% to about 10%, the amount of castor oil is about 0.01% to about 1.5%, the amount of povidone is about 0.05% to about 1%, the amount of tartaric acid is about 0% to about 1%, and the amount of magnesium stearate is about 0% to about 2%.

118. A niacin microcapsule having a coating ratio of about 10% to about 30%, wherein the amount of nicotinic acid is about 60% to about 90%, the amount of methacrylic acid copolymer type C (L100-55) is about 0% to about 15%, the amount of methacrylic acid copolymer type B (S100) is about 0% to about 15%, and the amount of cottonseed oil is about 2% to about 15%.
